SEQUENCE LISTING (77 sequences)

SEQ ID NO: 1 - DNA, 20 bp, Artificial Sequence
  gRNA
gctggtgcaagaccccgagt

SEQ ID NO: 2 - DNA, 20 bp, Artificial Sequence
  gRNA
caagatccctttcattcgag

SEQ ID NO: 3 - DNA, 20 bp, Artificial Sequence
  gRNA
tggtgtggtcgcagatcgag

SEQ ID NO: 4 - DNA, 20 bp, Artificial Sequence
  gRNA
aggaggcgattcccagctcg

SEQ ID NO: 5 - DNA, 20 bp, Artificial Sequence
  gRNA
cgagaacacggaagcgctgc

SEQ ID NO: 6 - DNA, 20 bp, Artificial Sequence
  gRNA
tggctaagctgagtgcctct

SEQ ID NO: 7 - DNA, 20 bp, Artificial Sequence
  gRNA
atgtcgtccagcaagctcag

SEQ ID NO: 8 - DNA, 20 bp, Artificial Sequence
  gRNA
tgtagtcgatgacgcgctgt

SEQ ID NO: 9 - DNA, 20 bp, Artificial Sequence
  gRNA
tggccagactgcgttccgac

SEQ ID NO: 10 - DNA, 20 bp, Artificial Sequence
  gRNA
ggtgcgcggctgctacgagg

SEQ ID NO: 11 - protein, 485 aa, Artificial Sequence
  Human anti-MSLN CAR
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 21 aa, Artificial Sequence
  CD8 Leader
MALPVTALLLPLALLLHAARP

SEQ ID NO: 13 - protein, 241 aa, Artificial Sequence
  M5 scFv
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 14 - protein, 45 aa, Artificial Sequence
  CD8 hinge
TTTPAPRPPTPAPTIASQPLSLRPEACRPAAGGAVHTRGLDFACD

SEQ ID NO: 15 - protein, 24 aa, Artificial Sequence
  CD8 transmembrane domain
IYIWAPLAGTCGVLLLSLVITLYC

SEQ ID NO: 16 - protein, 42 aa, Artificial Sequence
  4-1BB ICD
KRGRKKLLYIFKQPFMRPVQTTQEEDGCSCRFPEEEEGGCEL

SEQ ID NO: 17 - protein, 112 aa, Artificial Sequence
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - DNA, 1455 bp, Artificial Sequence
  Human anti-MSLN CAR
atggccctccctgtcaccgccctgctgcttccgctggctcttctgctccacgccgctcggccccaagtccaactcgttcaatcaggcgcagaagtcgaaaagcccggagcatcagtcaaagtctcttgcaaggcttccggctacaccttcacggactactacatgcactgggtgcgccaggctccaggccagggactggagtggatgggatggatcaacccgaattccgggggaactaactacgcccagaagtttcagggccgggtgactatgactcgcgatacctcgatctcgactgcgtacatggagctcagccgcctccggtcggacgataccgccgtgtactattgtgcgtcgggatgggacttcgactactgggggcagggcactctggtcactgtgtcaagcggaggaggtggatcaggtggaggtggaagcgggggaggaggttccggcggcggaggatcagatatcgtgatgacgcaatcgccttcctcgttgtccgcatccgtgggagacagggtgaccattacttgcagagcgtcccagtccattcggtactacctgtcgtggtaccagcagaagccggggaaagccccaaaactgcttatctatactgcctcgatcctccaaaacggcgtgccatcaagattcagcggttcgggcagcgggaccgactttaccctgactatcagcagcctgcagccggaagatttcgccacgtactactgcctgcaaacctacaccaccccggacttcggacctggaaccaaggtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 19 - DNA, 63 bp, Artificial Sequence
  CD8 Leader
atggccctccctgtcaccgccctgctgcttccgctggctcttctgctccacgccgctcggccc

SEQ ID NO: 20 - DNA, 723 bp, Artificial Sequence
  M5 scFv
caagtccaactcgttcaatcaggcgcagaagtcgaaaagcccggagcatcagtcaaagtctcttgcaaggcttccggctacaccttcacggactactacatgcactgggtgcgccaggctccaggccagggactggagtggatgggatggatcaacccgaattccgggggaactaactacgcccagaagtttcagggccgggtgactatgactcgcgatacctcgatctcgactgcgtacatggagctcagccgcctccggtcggacgataccgccgtgtactattgtgcgtcgggatgggacttcgactactgggggcagggcactctggtcactgtgtcaagcggaggaggtggatcaggtggaggtggaagcgggggaggaggttccggcggcggaggatcagatatcgtgatgacgcaatcgccttcctcgttgtccgcatccgtgggagacagggtgaccattacttgcagagcgtcccagtccattcggtactacctgtcgtggtaccagcagaagccggggaaagccccaaaactgcttatctatactgcctcgatcctccaaaacggcgtgccatcaagattcagcggttcgggcagcgggaccgactttaccctgactatcagcagcctgcagccggaagatttcgccacgtactactgcctgcaaacctacaccaccccggacttcggacctggaaccaaggtggagatcaag

SEQ ID NO: 21 - DNA, 135 bp, Artificial Sequence
  CD8 hinge
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 22 - DNA, 72 bp, Artificial Sequence
  CD8 transmembrane domain
atctacatttgggcccctctggctggtacttgcggggtcctgctgctttcactcgtgatcactctttactgt

SEQ ID NO: 23 - DNA, 126 bp, Artificial Sequence
  4-1BB ICD
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 24 - DNA, 336 bp, Artificial Sequence
  CD3 zeta
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 25 - protein, 488 aa, Artificial Sequence
  Human anti-CD19 CAR
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 242 aa, Artificial Sequence
  hCD19 scFv
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 27 - protein, 27 aa, Artificial Sequence
  CD28 transmembrane domain
FWVLVVVGGVLACYSLLVTVAFIIFWV

SEQ ID NO: 28 - protein, 41 aa, Artificial Sequence
  CD28 ICD
RSKRSRLLHSDYMNMTPRRPGPTRKHYQPYAPPRDFAAYRS

SEQ ID NO: 29 - protein, 112 aa, Artificial Sequence
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - DNA, 1464 bp, Artificial Sequence
  Human anti-CD19 CAR
atggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 31 - DNA, 63 bp, Artificial Sequence
  CD8 leader
atggccttaccagtgaccgccttgctcctgccgctggccttgctgctccacgccgccaggccg

SEQ ID NO: 32 - DNA, 726 bp, Artificial Sequence
  hCD19 scFv
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 33 - DNA, 135 bp, Artificial Sequence
  CD8 hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 34 - DNA, 81 bp, Artificial Sequence
  CD28 transmembrane domain
ttttgggtgctggtggtggttggtggagtcctggcttgctatagcttgctagtaacagtggcctttattattttctgggtg

SEQ ID NO: 35 - DNA, 123 bp, Artificial Sequence
  CD28 ICD
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 36 - DNA, 336 bp, Artificial Sequence
  CD3 zeta
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - protein, 486 aa, Artificial Sequence
  Human anti-CD19 CAR
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 1458 bp, Artificial Sequence
  Human anti-CD19 CAR
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 39 - protein, 249 aa, Artificial Sequence
  Anti-GD2 scFv
GSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGGGGSGGGGSGGGGSGGGGSEVQLLQSGPELEKPSASVMISCKASGSSFTGYNMNWVRQNIGKSLEWIGAIDPYYGGTSYNQKFKGRATLTVDKSSSTAYMHLKSLTSEDSVYYCVSGMEYWGQGTSVTVSSSG

SEQ ID NO: 40 - DNA, 747 bp, Artificial Sequence
  Anti-GD2 scFv
ggatccgatgttgtgatgacccaaactccactctccctgcctgtcagtcttggagatcaagcctccatctcttgcagatctagtcagagtcttgtacaccgtaacggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacacgttcctccgctcacgttcggtgctgggaccaagctggagctgaaaggaggtggcgggtcagggggtggcggaagcggaggcggcggttcaggcggaggaggctcggaggtgcagcttctgcagtctggacctgagctggagaagccttccgcttcagtgatgatatcctgcaaggcttctggttcctccttcactggctacaacatgaactgggtgaggcagaatattggaaagagccttgaatggattggagctattgatccttactacggtggaactagctacaaccagaagttcaagggcagggccacattgactgtagacaaatcgtccagcacagcctacatgcacctcaagagcctgacatctgaggactctgtctattactgtgtaagcggaatggagtactggggtcaaggaacctcagtcaccgtctcctcatccgga

SEQ ID NO: 41 - protein, 245 aa, Artificial Sequence
  Anti-HER2 scFv
GDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSS

SEQ ID NO: 42 - DNA, 735 bp, Artificial Sequence
  Anti-HER2 scFv
ggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctttattctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggacgtgtggggtcaaggaaccctggtcaccgtctcctcg

SEQ ID NO: 43 - protein, 245 aa, Artificial Sequence
  Anti-HER2 scFv
GDIQMTQSPSSLSASVGDRVTITCRASQDVNTAVAWYQQKPGKAPKLLIYSASFLESGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFVAMDVWGQGTLVTVSS

SEQ ID NO: 44 - DNA, 735 bp, Artificial Sequence
  Anti-HER2 scFv
ggagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttccttgagtctggagtcccttctcgcttctctggatctagatctgggacggatttcactctgaccatcagcagtctgcagccggaagacttcgcaacttattactgtcagcaacattatactactcctcccacgttcggacagggtaccaaggtggagatcaaacgcactgggtctacatctggatctgggaagccgggttctggtgagggttctgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttcgttgctatggacgtgtggggtcaaggaaccctggtcaccgtctcctcg

SEQ ID NO: 45 - protein, 244 aa, Artificial Sequence
  Anti-TnMuc1 scFv
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 46 - DNA, 732 bp, Artificial Sequence
  Anti-TnMuc1 scFv
caggtgcagctgcagcagtctgatgccgagctcgtgaagcctggcagcagcgtgaagatcagctgcaaggccagcggctacaccttcaccgaccacgccatccactgggtcaagcagaagcctgagcagggcctggagtggatcggccacttcagccccggcaacaccgacatcaagtacaacgacaagttcaagggcaaggccaccctgaccgtggacagaagcagcagcaccgcctacatgcagctgaacagcctgaccagcgaggacagcgccgtgtacttctgcaagaccagcaccttctttttcgactactggggccagggcacaaccctgacagtgtctagcggaggcggaggatctggcggcggaggaagtggcggagggggatctgaactcgtgatgacccagagccccagctctctgacagtgacagccggcgagaaagtgaccatgatctgcaagtcctcccagagcctgctgaactccggcgaccagaagaactacctgacctggtatcagcagaaacccggccagccccccaagctgctgatcttttgggccagcacccgggaaagcggcgtgcccgatagattcacaggcagcggctccggcaccgactttaccctgaccatcagctccgtgcaggccgaggacctggccgtgtattactgccagaacgactacagctaccccctgaccttcggagccggcaccaagctggaactgaag

SEQ ID NO: 47 - protein, 243 aa, Artificial Sequence
  Anti-PSMA scFv
GSDIVMTQSHKFMSTSVGDRVSIICKASQDVGTAVDWYQQKPGQSPKLLIYWASTRHTGVPDRFTGSGSGTDFTLTITNVQSEDLADYFCQQYNSYPLTFGAGTMLDLKGGGGSGGGGSSGGGSEVQLQQSGPELVKPGTSVRISCKTSGYTFTEYTIHWVKQSHGKSLEWIGNINPNNGGTTYNQKFEDKATLTVDKSSSTAYMELRSLTSEDSAVYYCAAGWNFDYWGQGTTLTVSSASSG

SEQ ID NO: 48 - DNA, 729 bp, Artificial Sequence
  Anti-PSMA scFv
ggatctgacattgtgatgacccagtctcacaaattcatgtccacatcagtaggagacagggtcagcatcatctgtaaggccagtcaagatgtgggtactgctgtagactggtatcaacagaaaccaggacaatctcctaaactactgatttattgggcatccactcggcacactggagtccctgatcgcttcacaggcagtggatctgggacagacttcactctcaccattactaacgttcagtctgaagacttggcagattatttctgtcagcaatataacagctatcctctcacgttcggtgctgggaccatgctggacctgaaaggaggcggaggatctggcggcggaggaagttctggcggaggcagcgaggtgcagctgcagcagagcggacccgagctcgtgaagcctggaacaagcgtgcggatcagctgcaagaccagcggctacaccttcaccgagtacaccatccactgggtcaagcagtcccacggcaagagcctggagtggatcggcaatatcaaccccaacaacggcggcaccacctacaaccagaagttcgaggacaaggccaccctgaccgtggacaagagcagcagcaccgcctacatggaactgcggagcctgaccagcgaggacagcgccgtgtactattgtgccgccggttggaacttcgactactggggccagggcacaaccctgacagtgtctagcgctagctccgga

SEQ ID NO: 49 - protein, 246 aa, Artificial Sequence
  Anti-EGFRvIII scFv
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 50 - DNA, 738 bp, Artificial Sequence
  Anti-EGFRvIII scFv
gagattcagctcgtgcaatcgggagcggaagtcaagaagccaggagagtccttgcggatctcatgcaagggtagcggctttaacatcgaggattactacatccactgggtgaggcagatgccggggaagggactcgaatggatgggacggatcgacccagaaaacgacgaaactaagtacggtccgatcttccaaggccatgtgactattagcgccgatacttcaatcaataccgtgtatctgcaatggtcctcattgaaagcctcagataccgcgatgtactactgtgctttcagaggaggggtctactggggacagggaactaccgtgactgtctcgtccggcggaggcgggtcaggaggtggcggcagcggaggaggagggtccggcggaggtgggtccgacgtcgtgatgacccagagccctgacagcctggcagtgagcctgggcgaaagagctaccattaactgcaaatcgtcgcagagcctgctggactcggacggaaaaacgtacctcaattggctgcagcaaaagcctggccagccaccgaagcgccttatctcactggtgtcgaagctggattcgggagtgcccgatcgcttctccggctcgggatcgggtactgacttcaccctcactatctcctcgcttcaagcagaggacgtggccgtctactactgctggcagggaacccactttccgggaaccttcggcggagggacgaaagtggagatcaag

SEQ ID NO: 51 - protein, 243 aa, Artificial Sequence
  Anti-FAP scFv
QIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIKGGGGSGGGGSGGGGSQVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSS

SEQ ID NO: 52 - DNA, 729 bp, Artificial Sequence
  Anti-FAP scFv
caaattgttctcacccagtctccagcgctcatgtctgcttctccaggggagaaggtcaccatgacctgcactgccagctcaagtgttagttacatgtactggtaccagcagaagccacgatcctcccccaaaccctggatttttctcacctccaacctggcttctggagtccctgctcgcttcagtggccgtgggtctgggacctctttctctctcacaatcagcagcatggaggctgaagatgctgccacttattactgccagcagtggagtggttacccacccatcacattcggctcggggacaaagttggaaataaaaggtggaggtggcagcggaggaggtgggtccggcggtggaggaagccaggtccaactgcagcagcctggggctgaactggtaaagcctggggcttcagtgaagttgtcctgcaaggcgtctggctacaccatcaccagctactctctgcactgggtgaagcagaggcctggacaaggccttgagtggattggagagattaatcctgccaatggtgatcataacttcagtgagaagttcgagatcaaggccacactgactgtagacagctcctccaacacagcattcatgcaactcagcaggctgacatctgaggactctgcggtctattactgtacaagattggacgatagtaggttccactggtacttcgatgtctggggcgcagggaccacggtcaccgtctcctca

SEQ ID NO: 53 - protein, 5 aa, Artificial Sequence
  linker
  REPEAT (1)..(5): repeat n times, where n is an integer of 1
GSGGS

SEQ ID NO: 54 - protein, 4 aa, Artificial Sequence
  linker
  REPEAT (1)..(4): repeat n times, where n is an integer of 1
GGGS

SEQ ID NO: 55 - protein, 5 aa, Artificial Sequence
  linker
  REPEAT (1)..(5): repeat n times, where n is an integer of 1
GGGGS

SEQ ID NO: 56 - protein, 4 aa, Artificial Sequence
  linker
GGSG

SEQ ID NO: 57 - protein, 5 aa, Artificial Sequence
  linker
GGSGG

SEQ ID NO: 58 - protein, 5 aa, Artificial Sequence
  linker
GSGSG

SEQ ID NO: 59 - protein, 5 aa, Artificial Sequence
  linker
GSGGG

SEQ ID NO: 60 - protein, 5 aa, Artificial Sequence
  linker
GGGSG

SEQ ID NO: 61 - protein, 5 aa, Artificial Sequence
  linker
GSSSG

SEQ ID NO: 62 - protein, 5 aa, Artificial Sequence
  linker
GGGGS

SEQ ID NO: 63 - protein, 15 aa, Artificial Sequence
  linker
GGGGSGGGGSGGGGS

SEQ ID NO: 64 - DNA, 45 bp, Artificial Sequence
  linker
ggtggcggtggctcgggcggtggtgggtcgggtggcggcggatct

SEQ ID NO: 65 - protein, 5 aa, Artificial Sequence
  hinge
DKTHT

SEQ ID NO: 66 - protein, 4 aa, Artificial Sequence
  hinge
CPPC

SEQ ID NO: 67 - protein, 15 aa, Artificial Sequence
  hinge
CPEPKSCDTPPPCPR

SEQ ID NO: 68 - protein, 12 aa, Artificial Sequence
  hinge
ELKTPLGDTTHT

SEQ ID NO: 69 - protein, 12 aa, Artificial Sequence
  hinge
ELKTPLGDTTHT

SEQ ID NO: 70 - protein, 7 aa, Artificial Sequence
  hinge
KCCVDCP

SEQ ID NO: 71 - protein, 7 aa, Artificial Sequence
  hinge
KYGPPCP

SEQ ID NO: 72 - protein, 15 aa, Artificial Sequence
  hinge
EPKSCDKTHTCPPCP

SEQ ID NO: 73 - protein, 12 aa, Artificial Sequence
  hinge
ERKCCVECPPCP

SEQ ID NO: 74 - protein, 17 aa, Artificial Sequence
  hinge
ELKTPLGDTTHTCPRCP

SEQ ID NO: 75 - protein, 12 aa, Artificial Sequence
  hinge
SPNMVPHAHHAQ

SEQ ID NO: 76 - protein, 15 aa, Artificial Sequence
  hinge
EPKSCDKTYTCPPCP

SEQ ID NO: 77 - protein, 9 aa, Artificial Sequence
  NY-ESO-1
SLLMWITQC

(19) United States
(12) Patent Application Publication
(10) Pub. No.: US 2024/0148791 A1
(43) Pub. Date: May 9, 2024

(54) COMPOSITIONS AND METHODS FOR ASSESSING AND TREATING T CELL DYSFUNCTION

(71) Applicant: THE TRUSTEES OF THE UNIVERSITY OF PENNSYLVANIA, Philadelphia, PA (US)

(72) Inventors: Carl H. June, Merion Station, PA (US); Regina M. Young, Bryn Mawr, PA (US); Shelley L. Berger, Wayne, PA (US); Charly R. Good, Springfield, PA (US); M. Angela Aznar Gomez, Philadelphia, PA (US); Shunichiro Kuramitsu, Nagoya (JP)

(21) Appl. No.: 18/549,252

(22) PCT Filed: Mar. 8, 2022
(86) PCT No.: PCT/US22/19333

§ 371 Date: Sep. 6, 2023

Related U.S. Application Data
(60) Provisional application No. 63/158,313, filed on Mar. 8, 2021.

(51) Int. Cl. A61K 35/17; A61K 39/00; A61P 35/00; C07K 16/30; C12N 5/0783; C12N 9/22; C12N 15/11; C12N 15/86; C12Q 1/6886

(52) U.S. Cl. CPC A61K 35/17; A61K 39/4611; A61K 39/4631; A61K 39/4632; A61K 39/464499; A61P 35/00; C07K 16/30; C12N 5/0636; C12N 9/22; C12N 15/11; C12N 15/86; C12Q 1/6886; A61K 2239/13; A61K 2239/17; A61K 2239/21; A61K 2239/22; C12N 2310/20; C12N 2740/15043; C12Q 2600/106; C12Q 2600/158

(57) ABSTRACT

The present disclosure provides modified immune cells or precursors thereof (e.g., gene edited modified T cells) comprising a modification in an endogenous gene locus encoding SOX and/or ID3. Methods for assessing and treating T cell dysfunction are also provided.

### CROSS-REFERENCE TO RELATED APPLICATION

The present application is entitled to priority under 35 U.S.C. § 119(e) to U.S. Provisional Patent Application No. 63/158,313 filed Mar. 8, 2021, which is hereby incorporated by reference in its entirety herein.

### STATEMENT REGARDING FEDERALLY SPONSORED RESEARCH OR DEVELOPMENT

This invention was made with government support under CA232466 awarded by the National Institutes of Health. The government has certain rights in the invention.

### BACKGROUND OF THE INVENTION

T cell exhaustion is a differentiation state acquired when T cells are exposed to persistent antigen stimulation in the setting of chronic viral infection or in response to tumors. Failure to eliminate antigen results in a progressive loss of effector functions or dysregulation. Hallmarks of T cell exhaustion include reduced effector function, distinct epigenetic and transcriptional gene signatures, sustained expression of multiple inhibitory receptors, defective cytokine production, increased chemokine expression, and limited proliferative capacity. Examination of genes upregulated in exhausted CD8+ tumor-infiltrating lymphocytes (TILs) from patients and TILs from mouse models has led to the identification of genes that restrain tumor immunity, including LAYN, Tox, and Gata-3. Furthermore, genome-wide CRISPR Cas9 knock-out and knock-in screens in mouse and human CD8+ T cells revealed additional targets such as Mapk14, Dhx37, ZC3H12A, Ptpn2, SOSCS1, and TGFBR2 that modulate T cell function. Importantly, engineered CAR T cells also acquire an exhausted phenotype when they enter the tumor microenvironment (TME) in in vivo models, leading to the hypothesis that CAR T cell exhaustion/dysfunction is a major hurdle for CAR T cell therapy.

There is a need in the art for finding novel inducers of exhaustion or dysfunction in CAR T cells that, when disrupted, will allow the advancement of even more effective CAR T cell therapies designed to treat solid tumors. The present invention addresses this need.

### SUMMARY OF THE INVENTION

In one aspect, the disclosure provides a modified immune cell or precursor cell thereof, comprising a modification in an endogenous gene locus encoding SOX and/or ID3, wherein the modification is capable of downregulating gene expression of endogenous SOX and/or ID3.

In certain embodiments, the modified immune cell or precursor cell further comprises an exogenous T cell receptor (TCR) and/or chimeric antigen receptor (CAR) comprising affinity for an antigen on a target cell.

In certain embodiments, the modification is selected from the group consisting of a substitution, an insertion, a deletion, and an insertion/deletion. In certain embodiments, the modification is mediated by a CRISPR system comprising a CRISPR nuclease and a guide RNA. In certain embodiments, the modification is mediated by CRISPR/Cas9.

In certain embodiments, the guide RNA comprises a guide sequence that is sufficiently complementary with a target sequence in the endogenous gene locus encoding SOX and/or ID3. In certain embodiments, the guide RNA comprises the nucleic acid sequence set forth in any one of SEQ ID NOs: 1-10.

In certain embodiments, the exogenous TCR is selected from the group consisting of a wild-type TCR, a high affinity TCR, and a chimeric TCR.

In certain embodiments, the exogenous CAR comprises an antigen binding domain, a transmembrane domain, and an intracellular domain. In certain embodiments, the antigen binding domain is selected from the group consisting of an antibody, an scFv, and a Fab.

In certain embodiments, the exogenous CAR further comprises a hinge domain. In certain embodiments, the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

In certain embodiments, the exogenous CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the exogenous CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3. In certain embodiments, the exogenous CAR comprises an intracellular domain comprising an intracellular domain selected from the group consisting of cytoplasmic signaling domains of a human CD3 zeta chain, FcyRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

In certain embodiments, the antigen on a target cell is a tumor associated antigen (TAA).

In another aspect, the disclosure provides a modified immune cell or precursor cell thereof, comprising a nucleic acid capable of overexpressing endogenous SOX and/or ID3.

In certain embodiments, the modified immune cell or precursor cell further comprises an exogenous T cell receptor (TCR) and/or chimeric antigen receptor (CAR) comprising affinity for an antigen on a target cell. In certain embodiments, the exogenous TCR is selected from the group consisting of a wild-type TCR, a high affinity TCR, and a chimeric TCR.

In certain embodiments, the exogenous CAR comprises an antigen binding domain, a transmembrane domain, and an intracellular domain. In certain embodiments, the antigen binding domain is selected from the group consisting of an antibody, an scFv, and a Fab.

In certain embodiments, the exogenous CAR further comprises a hinge domain. In certain embodiments, the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

In certain embodiments, the exogenous CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the exogenous CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3. In certain embodiments, the exogenous CAR comprises an intracellular domain comprising an intracellular domain selected from the group consisting of cytoplasmic signaling domains of a human CD3 zeta chain, FcyRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

In certain embodiments, the antigen on a target cell is a tumor associated antigen (TAA).

In certain embodiments, the modified cell is resistant to cell exhaustion and/or dysfunction.

In certain embodiments, the modified cell is an autologous cell. In certain embodiments, the modified cell is a cell isolated from a human subject. In certain embodiments, the modified cell is a modified T cell. In certain embodiments, the modified cell is a modified T cell resistant to T cell exhaustion and/or T cell dysfunction.

In another aspect, the disclosure provides a method for generating a modified immune cell or precursor cell thereof. The method comprises introducing into an immune or precursor cell a CRISPR system comprising one or more polypeptides and/or nucleic acids capable of downregulating gene expression of endogenous SOX and/or ID3; and introducing into the immune or precursor cell a nucleic acid encoding an exogenous T cell receptor (TCR) and/or chimeric antigen receptor (CAR). The exogenous TCR and/or CAR comprises affinity for an antigen on a target cell.

In another aspect, the disclosure provides a method for generating a modified immune cell or precursor cell thereof. The method comprises introducing into an immune or precursor cell a nucleic acid capable of over-expressing endogenous SOX and/or ID3; and introducing into the immune or precursor cell a nucleic acid encoding an exogenous T cell receptor (TCR) and/or chimeric antigen receptor (CAR). The exogenous TCR and/or CAR comprises affinity for an antigen on a target cell.

In certain embodiments, the one or more polypeptides and/or nucleic acids capable of downregulating gene expression of endogenous SOX and/or ID3 introduces a CRISPR-mediated modification in an endogenous gene locus encoding SOX and/or ID3. In certain embodiments, the modification is selected from the group consisting of a substitution, an insertion, a deletion, and an insertion/deletion. In certain embodiments, the CRISPR system comprises a CRISPR nuclease and a guide RNA. In certain embodiments, the CRISPR nuclease is Cas9. In certain embodiments, the CRISPR nuclease and the guide RNA comprise a ribonucleoprotein (RNP) complex. In certain embodiments, the RNP complex is introduced by electroporation. In certain embodiments, the guide RNA comprises a guide sequence that is sufficiently complementary with a target sequence in the endogenous gene locus encoding SOX and/or ID3. In certain embodiments, the guide RNA comprises a nucleic acid sequence set forth in any one of SEQ ID NO: 1-10.

In certain embodiments, the nucleic acid encoding an exogenous TCR and/or CAR is introduced via viral transduction. In certain embodiments, the viral transduction comprises contacting the immune or precursor cell with a viral vector comprising the nucleic acid encoding an exogenous TCR and/or CAR. In certain embodiments, the viral vector is selected from the group consisting of a retroviral vector, a lentiviral vector, an adenoviral vector, and an adeno-associated viral (AAV) vector. In certain embodiments, the viral vector is a lentiviral vector. In certain embodiments, the viral transduction comprises contacting the immune or precursor cell with a viral vector comprising the nucleic acid encoding an exogenous TCR and/or CAR.

Another aspect of the disclosure provides a method of treating a disease or disorder in a subject in need thereof. The method comprises administering to the subject any of the modified immune or precursor cells contemplated herein, or a modified immune or precursor cell generated by any of the methods contemplated herein.

In another aspect, the disclosure provides a method of treating a disease or disorder in a subject in need thereof. The method comprises administering to the subject a modified T cell comprising a CRISPR-mediated modification in an endogenous gene locus encoding SOX and/or ID3, wherein the modification is capable of downregulating gene expression of endogenous SOX and/or ID3; and an exogenous T cell receptor (TCR) and/or chimeric antigen receptor (CAR) comprising affinity for an antigen on a target cell.

In another aspect, the disclosure provides a method of treating a disease or disorder in a subject in need thereof, comprising administering to the subject a modified T cell comprising a modification in an endogenous gene locus encoding SOX and/or ID3, wherein the modification is capable of over-expressing endogenous SOX and/or ID3; and an exogenous T cell receptor (TCR) and/or chimeric antigen receptor (CAR) comprising affinity for an antigen on a target cell.

In certain embodiments, the antigen on a target cell is a tumor associated antigen (TAA).

In certain embodiments, the disease or disorder is cancer. In certain embodiments, the cancer comprises a solid tumor.

In certain embodiments, the disease or disorder is a chronic infection. In certain embodiments, the chronic infection is selected from the group consisting of HIV, EBV, CMV, LCMV.

In certain embodiments, the modified T cell is human. In certain embodiments, the modified T cell is autologous. In certain embodiments, the subject is human.

Another aspect of the disclosure provides a method of assessing T cell dysfunction in a subject. The method comprises measuring a panel of genes in a sample from the subject. The panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY, wherein when at least 11 of the genes are upregulated, the T cell is dysfunctional.

In certain embodiments, the T cell comprises a CAR. In certain embodiments, the T cell comprises an engineered TCR. In certain embodiments, the CAR or TCR is capable of binding a tumor associated antigen (TAA).

In another aspect, the disclosure provides a method for treating cancer in a subject in need thereof. The method comprises i) administering a CAR T cell therapy to the subject, and ii) measuring a panel of genes in a sample from the subject. The panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY. When at least 11 of the genes are upregulated, the CAR T cells are deemed dysfunctional and an alternative therapy is administered.

In another aspect, the disclosure provides a method of treating cancer in a subject in need thereof, comprising: i) administering to the subject a therapy comprising a T cell comprising an engineered TCR capable of binding a tumor associated antigen (TAA), and ii) measuring a panel of genes in a sample from the subject. The panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY. When at least 11 of the genes are upregulated, the T cells are deemed dysfunctional and an alternative therapy is administered.

In another aspect, the disclosure provides a method of treating a disease, disorder, or chronic infection in a subject in need thereof. The method comprises i) administering to the subject a T cell therapy, and ii) measuring a panel of genes in a sample from the subject. The panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY. When at least 11 of the genes are upregulated, the cells are deemed dysfunctional and an alternative therapy is administered.

In certain embodiments, the chronic infection is selected from the group consisting of HIV, EBV and CMV.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIGS. 1A-1K: CAR T cell dysfunction develops during chronic antigenic stimulation with reversible loss of cell surface expression of the CAR in vitro and in patients. FIG. 1A: Experimental design of CAR T cell dysfunction in vitro model. T cells transduced with CAR directed against mesothelin (M5CAR) (day 0 product) are repeatedly stimulated with AsPC-1 cells. CAE (continuous antigen exposure) M5CAR T cells are sorted for further analyses. FIG. 1B: Population doubling level of M5CAR transduced T cells during CAE. Five normal donors (ND) were tested. FIG. 1C: Time-related changes in surface expression of M5CAR on CD8+ T cells. Data are presented as 6 individual experiments. FIG. 1D: Time related changes in percent of CD8+ CAR+ T cells expressing PDL-1 and CTLA-4 during CAE. Data from two donors are depicted. FIG. 1E: M5 CAR T cell lysis of AsPC-1 pancreatic tumor cell line before and after CAE. Sorted day 0 and day 28 CAE CD8+ surface CAR positive (surCARpos) T cells were co-cultured at 4:1 effector: target (E:T) ratio and a real-time, impedance-based assay was performed using xCelligence (ACEA Biosciences). Media and non-specific CD19BBz T cells are used as controls. Data are representative of 4 different donors (see Figure S1C). FIG. 1F: Cytokine profile of CD8+ surCAR pos T cells (day 28 CAE, day 0 product and control CD19BBz T cells). Sorted CD8+ surCAR pos T cells are co-cultured with AsPC-1 cells and cytokines in the supernatant were analyzed by high-sensitivity LUMINEX assay. ****P<0.0001 by two-way ANOVA with Tukey's post hoc test. Data are representative of 3 different donors (see Figure S1G. FIG. 1G: qPCR detection of M5CAR genomic DNA in CD8+ surCARpos T cells (left) and CD8+ surface CAR negative (surCARneg) T cells (right) on days 4, 7, and 17 of CAE for donor ND150. Y axis is average M5CAR copies per cell. FIG. 1H: Surface CAR expression on CAE CD8+ CAR T cells before and after rest with IL-15. CD8+ surCARneg T cells were sorted on day 23 (left) and surface CAR expression was examined after rest with IL-15 supplement for 24 hrs (right). FIG. 1I: IL-2 production. FIG. 1J: Cell killing capacity of CD8+M5CAR transduced T cells against AsPC-1 cells after 26 days of CAE before and after 24 hrs of rest with IL-15 (7:1 E:T ratio) measured by Celigo. Data are representative of 2 donors (see FIG. 9C). ****P<0.0001, ***P<0.001, **P<0.01 by Student's t test. % lysis=(1−count # of live target cells (GFP) in wells with effector cells/count # of live target cells (GFP) in wells without effector cells)×100. FIG. 1K: Surface M5CAR expression (top) and intracellular M5CAR expression (bottom) on human CD8+ T cells from pleural fluid 36 days post-M5CAR T cell infusion (patient #02916-06). M5CAR staining (right) and M5CAR FMO control (left; see FIG. 9H).

FIGS. 2A-2H: Transcriptional dynamics of dysfunctional CAR T cells. FIG. 2A: Identification of differentially expressed genes between day 0 and 28 CAE surCARpos cells. Genes on the right are upregulated at day 28 (N=521) and genes on the left are downregulated (N=517). Analysis includes four biological replicates (CAR T donors ND516, ND538, ND388, ND534). FIG. 2B: Average gene expression values (TPMs) for day 28 surCARpos compared to day 28 surCARneg for differentially expressed genes defined in FIG. 2A (top) and all genes (bottom). FIG. 2C: Ingenuity Pathway Analysis (IPA) of genes differentially expressed between day 0 and 28 (N=1,038 genes from FIG. 2A). A selection of pathways are shown, and a full list of pathways can be found in supplemental materials. FIG. 2D: Normalized RNA-seq counts of representative NK receptor/marker genes. Average of four biological replicates. FIG. 2E: Heatmap of genes differentially expressed between day 0, 16, and 28 CAE surCARpos cells (N=762 genes). Representative genes in each cluster are illustrated on the right. Average of two biological replicates (ND388, ND534). FIG. 2F: IPA upstream regulator analysis of transcription factors predicted to regulate the differentially expressed genes between day 0 and 28 (N=1,038 genes from FIG. 2A). Transcription factors are ranked by significance using the −log(pvalue). Only transcription factors that are differentially expressed in CAE are shown (p value <2e-5 and log₂ fold change >1). Gene expression log₂ fold change for each transcription factor is displayed on the right. Positive number indicates genes upregulated at day 28 and negative numbers are genes downregulated at day 28. FIG. 2G-2H: Representative ATAC-seq tracks (top) and pooled RNA-seq tracks (bottom) from day 0 and 28 samples at the ID3 (FIG. 2G) and KLF2 (FIG. 2H) regulatory regions. Analysis includes four biological replicates.

FIGS. 3A-3I: Single-cell analysis of CAE CD8+ T cells reveals co-expression of dysfunction signature genes. UMAP projection of single-cell gene expression data from day 0 CAR T product (FIG. 3A) and day 20 CAE cells (FIG. 3B) for donor ND388, made using Seurat. Each dot corresponds to one cell and cell clusters are color coded. (FIG. 3C) Heatmap of top 10 marker genes for each day 20 CAE cluster as defined in B. Columns correspond to cells and rows correspond to gene names. FIG. 3D: Gene ontology determined by metascape pathway analysis for each single-cell cluster from the day 20 CAE sample. Columns are cell clusters (from FIG. 3B) and rows are enriched pathways color coded by level of significance. FIG. 3E: Volcano plot depicting differentially expressed genes between day 20 CAE clusters 1 and 4 (dysfunctional) and clusters 2 and 3 (non-dysfunctional). Genes upregulated in the dysfunctional clusters are on the right side and genes downregulated are on the left. The x axis is log₂(fold change) and y axis is −log₁₀(p value). FIG. 3F: Dot plot illustrating the expression level of genes in day 0 (left) and day 20 CAE (right) samples, donor ND388. Genes included are dysfunction signature genes, naïve/memory, cell cycle and control genes. Each column represents one cluster as depicted in FIG. 3A (day 0) and FIG. 3B (day 20). The size of the circle represents the percent of cells expressing the gene in each cluster and the color depicts how highly expressed the gene is within that cluster. Listed below are the number of cells found in each cluster. FIG. 3G: Heatmap of adjacency matrix values from gene regulatory network analysis (PIDC) for day 20 CAE cells. Columns and rows are the top 500 most variable genes determined by Seurat. Depicted on the right are select genes found within the same community, boxed in red. FIG. 3H: Normalized counts of CAR transcripts from single-cell data for day 20 and 28 CAE cells. Pooled cells from dysfunctional clusters and non-dysfunctional clusters from 3 independent experiments, using CAR T donors ND388, ND538, and ND150. FIG. 3I: Percentage of cells that express the CAR ligand in dysfunctional and non-dysfunctional clusters. Average of three independent experiments.

FIGS. 4A-4M: Mass and flow cytometry profiling reveals NK-like phenotype of CD8+ CAR T cells under CAE. FIG. 4A: Time-related changes in NK-associated molecules (CD94, NKG2A, NKG2C, TIGIT, CD161, CD56, and TCRVa24-Ja18) and PD-1 and CD28 on surCARpos and surCARneg CD8+ T cells during CAE by flow cytometry. FIG. 4B: Phenotypical change between day 0 products (top) and day 29 CAE samples (bottom) profiled by mass cytometry using a NK flow panel. Data from 2 donors (ND150 and ND538) are shown. FIG. 4C: Expression of surface M5CAR and NK-associated molecules (CD161, TIGIT, CD56, NKG2A, NKG2C) and granulysin on day 0 product (top) and day 29 CAE CD8+ T cells (bottom). Circles highlight subpopulations of CD8+ T cells more abundant under CAE. FIG. 4D: Experimental design of the recurrent AsPC-1 mouse model. FIG. 4E: AsPC-1 tumor growth volumes in M5CAR T-treated mice. Arrows indicate tumors analyzed after recurrence. FIG. 4F: NK-associated molecules expression in CD8 day 0 product (top) and TILs from a representative AsPC-1 recurrent tumor (bottom). FIG. 4G: Average expression of NK-associated molecules on CD8 T cells in day 0 product and in three recurrent tumors. Each datapoint represents a single mouse for recurrent tumor data and a single technical replicate staining for day 0 product. FIG. 4H: PD-1, LAG3, and TIM3 expression in CD8 day 0 product (top) and TILs from a representative AsPC-1 recurrent tumor (bottom). FIG. 4I: Average expression of checkpoint receptors PD-1, LAG3, and TIM3 in CD8 T cells. Each datapoint represents a single mouse for recurrent tumor data and a single technical replicate staining for day 0 product. FIG. 4J: CD56 expression in CD8+ surCARpos T cells isolated from DLBCL patients at the peak of CTL019 expansion. FIG. 4K: Expression of NK-associated molecules and PD-1 on CD8+ surCARpos T cells in day 0 product and day 27 peripheral blood T cells from a patient with DLBCL (#13413-39). FIG. 4L: Timeline showing the experimental design of NY-ESO-1 TIL mouse model. FIG. 4M: Heatmap of dysfunction signature genes in NY-ESO-1 reactive CD8+ TILs along with blood (CD8+CD45RO+ T cells) and day 0 infused product. Data from FIG. 4G and FIG. 4I are shown as mean±SEM, and significance was assessed by two-way ANOVA plus Sidak test. ****p<0.0001, ***p<0.001, **p<0.01, n.s.: not significant.

FIGS. 5A-5F: Transition of CD8+ T cells to NK-like T cells upon continuous antigen stimulation. FIG. 5A: NK-like T cells are specifically expressed in dysfunctional clusters. NK-like T cell population, depicted by co-expression of CD3, KLRB1, and KLRC1, at day 0 (left) and day 20 CAE (right) overlayed on UMAP graphs from FIGS. 3A-3B. FIG. 5B: Identification of NK-like T cell populations during CAE time course—CD56+CD3+(top) and CD3+ KLRB1+(bottom). FIG. 5C: On left, NK-like T cell frequency measured by flow cytometry (CD3+CD56+) at day 0 (top, control) and following CD56 depletion (bottom). NK-like T cell frequency (CD3+CD56+) with or without CD56 depletion during CAE (right). FIG. 5D: Single-cell TCR fingerprinting+ gene expression analysis in ND150 (left) and ND538 (right). Results are filtered for CD8+ T cells that have the same CDR3 TCR sequence at day 0 and at day 28. Cells were classified as either KLRB1 negative or positive at day 0 and at day 28 and total number of cells in each category is depicted. FIG. 5E: Monocle trajectory analysis of ND388 day 20 CAE cells, with single-cell clusters labeled according to their defined clusters in FIG. 3B (left). On right, same monocle trajectory but with cells labeled according to expression of the dysfunction gene signature (N=30 genes, see FIG. 3F). FIG. 5F: Monocle trajectory analysis of ND150 and ND538 day 0 and day 28 CAE cells combined, corresponding to supplemental FIGS. 13 and 14. Cells are labeled according to sample ID (left) or by how highly each cell expresses the dysfunction signature genes (right).

FIGS. 6A-6I: ID3 and SOX4 are potential regulators of the dysfunction signature. FIG. 6A: Select transcription factors predicted to regulate differentially expressed genes between day 0 and day 20 CAE cells in single-cell sequencing datasets, identified using IPA upstream regulator analysis software. Depicted are transcription factors that overlap with factors from FIG. 2F. X axis is −log(p value) of transcription factor enrichment. On right, gene expression log₂ fold change (day 20 CAE/day 0) for each transcription factor—calculated as the number of cells at day 20 that upregulate or downregulate a gene compared to day 0 cells, as determined by cellfishing.jl software. NA depicts genes that are not differentially expressed between day 0 and day 20 cells. FIG. 6B: Single-cell transcript levels of ID3 and SOX4 illustrated by UMAP plots, corresponding to clusters from FIG. 3B (day 20 CAE cells). Top two clusters are dysfunctional. FIG. 6C: Violin plots depicting gene expression levels for ID3 and SOX4 for each cluster from day 20 CAE cells (see FIG. 3B). FIG. 6D: Single-cell transcript levels of CDKN2A, BCL6, RBPJ, ID2, and KLF2 illustrated by UMAP plots, corresponding to clusters from FIG. 3B (day 20 CAE cells). FIG. 6E: HOMER motif analysis depicting top 10 enriched transcription factor motifs in polyclonal ATAC-seq dataset for day 0 samples (left) and day 28 samples (right). Analysis includes four biological replicates (CAR T donors ND516, ND538, ND388, and ND534). FIG. 6F: Box plots illustrating the ATAC-seq signal at peaks that are not changed between day 0 and day 28 (left) and peaks that are specific to day 28 (right). The data are further subdivided into peaks that have (or do not have) an underlying SOX4 motif FIG. 6G-61: ATAC-seq tracks in regulatory regions at SOX4 motifs from day 0 and 28 CAE samples at dysfunction genes AFAP1L2 (FIG. 6G), CDK6 (FIG. 6H), and CSF1 (FIG. 6I). SOX4 motifs labeled with bars above tracks. Analysis includes four biological replicates.

FIGS. 7A-7C: In vivo relevance of CAR T dysfunction signature. FIG. 7A: NY-ESO-1 Ly95 TCR-specific TILs upregulate most genes in CAR T dysfunction signature. Heatmap of dysfunction signature genes (N=30), in NY-ESO-1 reactive CD8+ TILs obtained from a lung xenograft tumor model, along with controls including day 40 blood (CD8+CD45RO+ T cells) and day 0 infused product. TILs were extracted 40 days following infusion and gene expression profiles (along with controls) were determined by microarray analysis. FIG. 7B: DLBCL patients treated with CTL019 express NK-like CAR T cells. CD8+ surCARpos T cells isolated from DLBCL patients at the peak CTL019 expansion were assayed for CD56+ expression. FIG. 7C: Expression of NK-associated molecules (NKG2A, CD94, CD56 and KLRB1) and PD-1 on CD8+ surCARpos T cells in day 0 product and day 27 peripheral blood T cells from a patient with DLBCL (#02916-39).

FIGS. 8A-8H: FIG. 8A: Detailed experimental design of CAR T cell dysfunction in vitro model. FIG. 8B: Mesothelin expression on AsPC-1 cells, measured by flow cytometry. FIG. 8C: Tumor cytotoxicity of CD8+ surCARpos T cells (CAE and day 0 product) against AsPC-1 cells using 3 different donor T cells (ND388, ND534, and ND516) measured by xCelligence. FIG. 8D: Cell killing capacity of CD8+M5CAR transduced T cells (CAE and day 0 product) against AsPC-1 cells using 2 different donor T cells (ND150 8:1 E:T ratio, ND538 7:1 E:T ratio) measured by Celigo. FIG. 8E: Mesothelin expression on mesothelin transduced K562 cells (K562-meso), measured by flow cytometry. FIG. 8F: Kinetics of K562-meso cell lysis incubated with CD8+M5CAR transduced T cells (day 26 CAE and day 0 product) using 2 different donor T cells (ND150 8:1 E:T ratio, ND538 7:1 E:T ratio) measured by Celigo. FIG. 8G: Cytokine secretion of CD8+ surCARpos T cells (day 28 CAE and day 0 product) stimulated with AsPC-1 for 24 hrs using 2 different donor T cells (ND388 and ND534). CD19BBz CAR T cells were tested as a control for allogeneic recognition of AsPC-1. ****P<0.0001, *P<0.05 by two-way ANOVA with Tukey's post hoc test. FIG. 8H: Cytokine profile of CD8+ surCARpos T cells (day 24 CAE and day 0 product). Sorted CD8+ surCARpos T cells were stimulated with PMA+ ionomycin or AsPC-1, and media was used as a control.

FIGS. 9A-9H: FIG. 9A: Surface CAR expression on day 23 CAE CD8+M5CAR transduced T cells before sorting, associated with FIG. 1H. FIG. 9B: Frequency of surCARpos CD8+ T cells used for cell killing assay (day 26), associated with FIG. 1J. FIG. 9C: Cell killing capacity of donor ND538 CD8+M5CAR transduced T cells against AsPC-1 cells after 26 days of CAE before and after 24 hrs of rest with IL-15 (7:1 E:T ratio). **P<0.01, *P<0.05 by Student's t test. FIG. 9D: Scheme of the experimental design to examine the effect of rest and cytokines on cell killing capacity and surface CAR expression. FIG. 9E: Frequency of residual tumor cells (EpCAM+CD45−) and T cells (EpCAM-CD45+) after coculture with CAE (top left), CAE+IL-7+IL-15 (top right), CAE+rest (bottom left), CAE+rest+IL-7+IL-15 (bottom right) on day 25. FIG. 9F: Effect of rest or IL-7+IL-15 on surface CAR expression on T cells in 3 donors. *P<0.05 by one-way ANOVA with Tukey's post hoc test. FIG. 9G: Tumor cells (mesothelin+CD45−) in human pleural fluid on day 36 after CAR T cell infusion (patient #02916-06) and in peritoneal fluid on day 21 after CAR T cell infusion (patient #02916-01). FIG. 9H: Surface CAR expression (top) and intracellular M5CAR expression (bottom) on human CD8+ T cells in peritoneal fluid (patient #02916-01) after 21 days of M5CAR T cell infusion. CAR staining (right) and M5CAR FMO control (left).

FIGS. 10A-10I: FIG. 10A: Venn diagram displaying overlap between genes upregulated in day 28 CAE surCARpos CD8+ T cells (see FIG. 2A) and genes upregulated in exhausted CD8+ T cells from the LCMV clone-13 mouse model of chronic viral infection. Only genes with mouse to human orthologs were included. FIG. 10B: Overlap of genes downregulated in day 28 CAE surCARpos cells (see FIG. 2A) and genes downregulated in exhausted T cells from the LCMV clone-13 mouse model of chronic viral infection. Overlap of genes upregulated in day 28 CAE surCARpos cells (see FIG. 2A) and genes that define dysfunctional CD8+ TILs from hepatocellular carcinoma patients [HCC] (FIG. 10C), melanoma patients (FIG. 10D), non-small-cell lung cancer patients [NSCLC] (FIG. 10E), and colorectal cancer patients [CRC] (FIG. 10F). FIG. 10G: Overlap of HCC, melanoma, NSCLC, and CRC dysfunctional TIL signature genes. FIG. 10H: Average gene expression values (TPMs) for differentially expressed genes defined in FIG. 2A. Gene expression values for day 0 surCARpos compared to day 0 surCARneg (left) and day 28 surCARpos compared to day 28 surCARneg (right). FIG. 10I: Average gene expression values (TPMs) for all genes. Gene expression values for day 0 surCARpos compared to day 0 surCARneg (left) and day 28 surCARpos compared to day 28 surCARneg (right).

FIGS. 11A-11G: FIG. 11A: ATAC-seq open chromatin regions specific to day 0 (left) or day 28 CAE (right) surCARpos cells. FIG. 11B: Genomic location of open chromatin regions for day 0 and day 28 CAE surCARpos cells. FIG. 11C: Relation of gene expression and chromatin changes during CAE. Average ATAC-seq signal of genes upregulated at day 28 (left) and genes downregulated (right) in day 0 and day 28 CAE surCARpos cells. Average of 4 biological replicates. FIG. 11D: Decile plot showing correlation between polyclonal RNA-seq and single-cell RNA-seq datasets. Genes differentially expressed in single-cell data between day 20 CAE and day 0, defined by cellfishing.jl, were divided into 10 groups and sorted by single-cell fold change, going from lowest to highest (x axis). Y-axis plots the fold change (day 28 CAE/day 0) in the polyclonal RNA-seq for genes in each group. FIG. 11E: Heatmap of top 10 marker genes for each day 0 single-cell cluster as defined in FIG. 3A, donor ND388. Columns correspond to cells and rows correspond to gene names. FIG. 11F: Gene ontology determined by metascape pathway analysis for each single-cell day 0 cluster, donor ND388. Columns are cell clusters (defined in FIG. 3A) and rows are enriched pathways color coded by level of significance. FIG. 11G: Violin plots depicting gene expression levels from day 20 CAE cells (donor ND388) for SRGAP3, DUSP4, CSF1, IL2RA, GZMB and CDK6. X axis is cell clusters defined in FIG. 3B.

FIGS. 12A-12E: FIG. 12A: UMAP plots depicting gene expression levels from day 0 cells (top) and 20 CAE cells (bottom) for dysfunction genes CD9, LAYN and RGS16, donor ND388, associated with UMAPs from FIGS. 3A and 3B. FIG. 12B: UMAP plots depicting gene expression levels from day 0 cells (top) and 20 CAE cells (bottom) for naïve/memory genes IL7R, SELL, KLF2 and TCF7 (donor ND388). FIG. 12C: Violin plots depicting gene expression levels from day 20 CAE cells for exhaustion genes HAVCR2, LAYN, and TNFRSF9 and NK associated genes KLRB1 and KLRC1 for donor ND388. X axis is cell clusters defined in FIG. 3B. FIG. 12D: Violin plot of day 20 CAE cells for CTLA4, donor ND388. FIG. 12E: Violin plots for T cell dysfunction genes NDFIP2, RGS16, and CD9 for day 20 CAE cells, donor ND388.

FIGS. 13A-13E: Single-cell analysis of donor ND538 day 0 product and day 28 CAE cells. FIG. 13A: UMAP projection of single-cell gene expression data from day 0 cells, made using Seurat. Each dot corresponds to one cell and cell clusters are color coded. FIG. 13B: UMAP projection of single-cell gene expression data from day 28 CAE T cells, made using Seurat. Each dot corresponds to one cell and cell clusters are color coded. FIG. 13C: Heatmap of top 10 marker genes for each day 28 CAE cluster as defined in FIG. 13B. Columns correspond to cells and rows correspond to gene names. FIG. 13D: Volcano plot depicting differentially expressed genes between day 28 CAE cluster 1 (dysfunctional) and clusters 2 and 3 (non-dysfunctional), also see FIG. 13E. Genes upregulated in the dysfunctional cluster are on the right side and genes downregulated are on the left. The x-axis is log₂(fold change) and y-axis is −log₁₀(pvalue). FIG. 13E: Dot plot illustrating the expression level of genes in day 0 (left) and day 28 CAE cell clusters (right). Genes included are dysfunction signature genes (N=30), naïve/memory, cell cycle, and control genes. Each column represents one cluster. The size of the circle represents the percent of cells expressing the gene in each cluster and the color depicts how highly expressed the gene is within that cluster. The number of cells in each cluster is written beneath cluster identity.

FIGS. 14A-14E: Single-cell analysis of donor ND150 day 0 product and day 28 CAE cells. FIG. 14A: UMAP projection of single-cell gene expression data from day 0 cells, made using Seurat. Each dot corresponds to one cell and cell clusters are color coded. FIG. 14B: UMAP projection of single-cell gene expression data from day 28 CAE cells, made using Seurat. Each dot corresponds to one cell and cell clusters are color coded. FIG. 14C: Heatmap of top 10 marker genes for each day 28 CAE cluster as defined in FIG. 14B. Columns correspond to cells and rows correspond to gene names. FIG. 14D: Volcano plot depicting differentially expressed genes between day 28 cluster 3 (non-dysfunctional) and clusters 1,2,4, and 5 (dysfunctional), also see FIG. 14E. Genes upregulated in the dysfunctional clusters are on the right side and genes downregulated are on the left. The x axis is log₂(fold change) and y axis is −log₁₀(pvalue). FIG. 14E: Dot plot illustrating the expression level of genes in day 0 (left) and day 28 CAE (right). Genes included are dysfunction signature genes (N=30), naïve/memory, cell cycle and control genes. Each column represents one cluster. The size of the circle represents the percent of cells expressing the gene in each cluster and the color depicts how highly expressed the gene is within that cluster. The number of cells in each cluster is written beneath cluster identity.

FIGS. 15A-15C: FIG. 15A: Model for CD56 depletion assay. Expected percentage of NK-like T cells (CD56+, y-axis) and time (x-axis) during continuous antigen stimulation for out competition model (left) and transition model (right). Control cells start with regular CAR T cell population at day 0 and CD56-depleted starts with day 0 CAR T cells depleted of CD56. FIG. 15B: TCR single-cell sequencing data for day 0 and day 28 CAE for donors ND150 and ND538. Y axis is the percentage of CDR3 sequences, and x-axis is the number of cells that have that CDR3 sequence. Illustrates that between 96-99% of CDR3 sequences are unique. FIG. 15C: Transcription factor motif for SOX4 from Jaspar database (top) and SOX4 and SOX17 motifs from UniProt database (bottom).

FIG. 16: Table of dysfunction signature genes (n=30) and whether they are found in vivo published datasets in exhausted T cells or dysfunction human TILs. HCC (hepatocellular carcinoma), NSCLC (non-small-cell lung cancer), CRC (colorectal carcinoma). X marks genes that are upregulated in LCMV exhausted T cells or defines dysfunctional TILs.

FIG. 17: sgRNAs for SOX4 (SEQ ID NOs: 1-5) and ID3 (SEQ ID NOs: 6-10).

FIGS. 18A-18B: CRISPR-mediated knock out of ID3 and SOX4 does not modify CAR T cell killing efficiency on Day 0 product but restores the killing ability of exhausted CAR T cells. FIG. 18A: Cell killing capacity of Day 0 product of ND539 MockM5.pTRPE, ID3KO.M5.pTRPE and SOX4.KO.M5.pTRPE CAR T cells against AsPC-1 cells measured by xCelligence. Day 0 CAR T cells were seeded in three different surface CAR+:Target ratios. Negative control (AsPC-1 only) is shown in grey. FIG. 18B: Cell killing capacity of ND539 MockM5.pTRPE, ID3KO.M5.pTRPE and SOX4.KO.M5.pTRPE CAR T cells against AsPC-1 cells after 18 days of CAE measured by xCelligence. Day 18 CAR T cells were seeded in 1:8 surface CAR+: target (left panel), 1:8 Total CAR+:Target (middle panel) and 1:8 CD45+:Target ratios. Negative control (AsPC-1 only) and positive control (Day 0 ND539 Mock M5.pTRPE product) are depicted.

FIGS. 19A-19C: Resting M5 CAR T and NY-ESO-1 TCR specific T cells results in the downregulation of 11/30 dysfunction signature genes. FIG. 19A: Heatmap of dysfunction signature genes (N=30) in Day 0 CAR T product, continuously stimulated M5 CAR T cells and rested M5 CAR T cells. Resting the continuously stimulated CAR T cells for 24 hours with IL-15 improves cytotoxic function (see FIG. 1J) The rested CAR T cells downregulate 15/30 of the dysfunction signature genes (compare ND388_pre_rest_CARpos to ND388_post_rest_CARpos). Genes downregulated with rest are denoted by a black bracket on the right. FIG. 19B: Heatmap of dysfunction signature genes (N=30), in NY-ESO-1 reactive CD8+ TILs obtained from a lung xenograft tumor model without rest (TIL_TCR) and with rest (TIL_TCR_rest), along with controls including day 40 blood (CD8+CD45RO+ T cells) and day 0 infused product. TILs were extracted 40 days following infusion and gene expression profiles (along with controls) were determined by microarray analysis. Rested NY-ESO-1 TCR specific TILs display improved cytotoxicity following rest (Moon et al., 2016) and downregulate 15/30 of the dysfunction signature genes (see black bracket on the right). FIG. 19C: Venn diagram overlap of genes going down with rest in M5 CAR T cells (left) and NY-ESO-1 TCR specific T cells (right). 11/30 (37%) of the dysfunction signature genes go down in both models with rest.

FIGS. 20A-20U: Disruption of ID3 and SOX4 improves CAR T effector function. FIG. 20A: Schematic representation of the CRISPR strategy to generate ID3 and SOX4 KO M5CAR T cells. FIG. 20B: Experimental design for WT, ID3 KO, and SOX4 KO analyses for donors ND566 and ND539. FIG. 20C: Agarose gel showing ID3 and SOX4 KO detection on cDNA from CD8 sorted populations after CAE for donor ND566. ID3: ID3 PCR; SOX4: SOX4 PCR; Positive Control: histone H3.3; WT: Mock M5CAR; W: water negative control; KO: ID3 KO (in ID3 PCR) and SOX4 KO (in SOX4 PCR). FIG. 20D: KO quantification of ID3 (ND566 and ND539) and SOX4 (ND566) by cDNA sequencing. Percent indels and fragment deletions upon CAE are shown as mean with standard deviation. FIG. 20E: UMAP projection of scRNA-seq data from sorted CD8+WT, ID3 KO, or SOX4 KO day 24 CAE cells for donor ND566-cells are color-coded by KO status. FIG. 20F: NK-like T cell population at day 24 CAE for donor ND566, depicted by co-expression of CD3, KLRB1, and KLRC1, overlayed on UMAP graphs from FIG. 20E. FIG. 20G: Percentage of NK-like T cells in WT, ID3 KO, and SOX4 KO cells, relative to WT (donor ND566). Significance by Fisher's exact test. FIG. 20H: UMAP graph from FIG. 7E with cells labeled according to expression of the dysfunction gene signature for donor ND566. FIGS. 20I-20J: Dysfunction score for WT, ID3 KO, and SOX4 KO cells for donor ND566 (FIG. 20I) and WT and ID3 KO cells for donor ND539 (FIG. 20J). Significance measured by Mann-Whitney U test. FIG. 20K: Dot plot illustrating the expression level of dysfunction signature genes in WT, ID3 KO, and SOX4 KO day 24 CAE cells, donor ND566. FIGS. 20L-20T: Violin plots depicting gene expression levels from WT, ID3 KO, and SOX4 KO day 24 CAE cells for SOX4 (FIG. 20L), AFAP1L2 (FIG. 20M), CSF1 (FIG. 20N), ID3 (FIG. 20O), LAYN (FIG. 20P), CD9 (FIG. 20Q), TNFRSF18 (R), GNLY (S), and KLRC1 (FIG. 20T) for donor ND566. FIG. 20U: Cell killing capacity of WT, ID3 KO, and SOX4 KO M5CAR T CAE cells, with controls media alone and day 0 CAR T product. Cells were collected and seeded at 1:8 E:T ratio with AsPC-1 on day 18 (ND539) and day 21 (ND566). Data are presented as mean±SEM. Significance by two-way ANOVA with Geisser-Greenhouse correction and Dunnet's post hoc test.

FIGS. 21A-21F: Detection of CAR T cell dysfunction in vivo. FIG. 21A: Experimental design of the recurrent AsPC-1 mouse model. FIG. 21B: AsPC-1 tumor growth volumes in M5CAR T-treated mice. Arrows indicate tumors analyzed. FIG. 21C: Representative plots showing MSLN expression from an AsPC-1 recurrent tumor. FIG. 21D: Frequency of CD8+ T cells infiltrating recurrent tumors. FIG. 21E: Frequency of CD8+ T cells expressing NK-associated molecules of our dysfunctional signature in day 0 product and in three recurrent tumors. FIG. 21F: Frequency of CD8+ T cells expressing PD-1, LAG3, and TIM3 in day 0 product and TILs. Fof panels FIG. 21E and FIG. 21F, each datapoint represents a single mouse for recurrent tumor data and a single technical replicate staining for day 0 product. Color code for mice data is matched with FIG. 21B.

FIGS. 22A-22C: SOX4 KO and ID3 KO CAR T cells elicit a superior antitumor response than M5 CAR T cell. FIG. 22A: Average tumor volumes (±SD) of AsPC-1 tumors treated with WT, ID3KO or SOX4KO M5 CAR T cells (ND539). Dashed lines indicate the day of infusion of CAR T cells (when tumors were at an average size of 250 mm³). Two-way Anova (Mixed-effects model) with Tukey's post-hoc test. FIG. 22B: Individual tumor volumes of (FIG. 22A). FIG. 22C: Summary of cured mice in two independent experiments at day 90. Experimental treatments and measurements were conducted in a blinded manner for both biological replicates (ND539 and ND561). Two-way Anova with Dunnet post-hoc test. Lines connect the experimental groups of each biological replicate.

FIGS. 23A-23I: Chromatin changes at NK receptor genes in dysfunctional CAR T cells, and ID3 and SOX4 KO CAR T cells have improved CAR T effector function. FIGS. 23A-23B: Representative ATAC-seq tracks in regulatory regions at SOX4 motifs from day 0 and day 28 CAE samples at NK receptor and dysfunction genes KLRC1 (FIG. 23A) and KLRB1 (FIG. 23B). SOX4 motifs labeled with bars above tracks. Analysis includes four biological replicates. FIG. 23C: Agarose gel (top) and KO efficiency by genomic DNA sequencing (bottom) showing CRISPR edits on KO T cells. ID3-specific and SOX4-specific PCR targeting the edited region of each transcription factor shows the appearance of two bands in KO UTD and KO M5CAR T cells, corresponding to two edited populations derived from different sgRNA hits as depicted in FIG. 20A. The KO efficiency quantification from two donors (ND566 and ND539). WT: wildtype, UTD: non-electroporated un-transduced T cells (no M5CAR), M5: T cells electroporated with M5CAR. Data is shown as mean±SEM from two CAR T donors. FIG. 23D: Cytotoxicity assessment of day 0 products at 1:1 (left) and 1:10 (right) E:T ratio on ND539 (top) and ND566 (bottom) in WT, ID3 KO, and SOX4 KO M5CAR T cells. Media used as a control. Data is shown as mean±SEM. FIG. 23E: Flow cytometry characterization of naive, central memory, effector memory and effector subsets on day 0 product with CD45RO and CCR7 expression for WT, ID3 KO, and SOX4 KO M5CAR T cells for donor ND566 (left) and ND539 (right). FIG. 23F: Percentage of NK-like T cells in WT and ID3 KO cells, relative to WT (donor ND539). Significance measured by Fisher's exact test. FIG. 23G: In vitro killing assay of ND539 WT, ID3 KO, and SOX4 KO M5CAR T cells. Cells were collected on day 18 of CAE and seeded at 1:8 E:T ratio with AsPC-1 on day 18. WT day 0 cells are used as a positive control and media is used as a negative control. Data is shown as mean±SEM. FIG. 23H: In vitro killing assay of ND566 WT, ID3 KO, and SOX4 KO M5CAR T cells. Cells were collected on day 21 of CAE and seeded at 1:8 E:T ratio. Data is shown as mean±SEM. FIG. 23I: In vitro killing assay of ND566 WT, ID3 KO, and SOX4 KO M5CAR T cells. Cells were collected on day 28 of CAE and seeded at 1:8 E:T ratio. Data is shown as mean±SEM. **p<0.01.

### DETAILED DESCRIPTION

The present disclosure provides compositions and methods for assessing and treating T cell dysfunction.

Chimeric antigen receptor (CAR) T cell therapy has achieved remarkable success in hematological malignancies but remains largely ineffective in solid tumors. A major factor leading to the reduced efficacy of CAR T cell therapy is T cell dysfunction, and the mechanisms mediating this dysfunction are under investigation. Herein, a robust model was establish to study mesothelin-redirected CAR T cell dysfunction in pancreatic cancer. Continuous antigen exposure results in hallmark features of exhaustion including reduced proliferation capacity and cytotoxicity, and severe defects in cytokine production. A transcriptional signature was identified at both population and single-cell levels in CAR T cells after continuous antigen exposure. In addition, TCR lineage tracing revealed a CD8+T-to-NK-like T cell plasticity that results in reduced tumor cell killing. The transcription factors SOX4 and ID3 are specifically expressed in the dysfunctional CAR NK-like T cells and are predicted to be master regulators of the dysfunction gene expression signature and the post-thymic acquisition of an NK-like T cell fate. The emergence of NK-like CAR T cells was identified in a subset of patients after infusion of CAR T cells. The findings gleaned from this study shed light on the plasticity of human CAR T cells and provide new approaches to improve the efficacy of CAR T cell therapy in solid tumors by preventing or revitalizing CAR T cell dysfunction.

In one aspect, the present disclosure provides compositions and methods for modified immune cells or precursors thereof (e.g., modified T cells) comprising a modification in an endogenous gene locus encoding SOX and/or ID3, and an exogenous (e.g., recombinant, transgenic or engineered) T cell receptor (TCR) and/or chimeric antigen receptor (CAR). In some embodiments, the modified immune cells are genetically edited such that the expression of SOX and/or ID3 is downregulated. In some embodiments, the modified immune cells are genetically edited such that SOX and/or ID3 is overexpressed. These genetically edited modified immune cells have enhanced immune function. In some embodiments, the genetically edited modified immune cells of the present disclosure are resistant to T cell exhaustion and/or dysfunction.

Also provided herein are methods for assessing T cell dysfunction. T cell dysfunction can be assessed in the context of a T cell therapy (e.g. CAR T cell therapy or TCR therapy). Methods of treatment in subjects receiving a T cell therapy are also provided, wherein T cell dysfunction is assessed in the subject and the subject is treated accordingly.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably +1%, and still more preferably +0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockin” as used herein refers to an exogenous nucleic acid sequence that has been inserted into a target sequence (e.g., endogenous gene locus). For example, a CAR/TCR knockin into a SOX and/or ID3 locus refers to a nucleic acid sequence encoding a chimeric antigen receptor (CAR) or T cell receptor (TCR) that has been inserted into a target location within the SOX and/or ID3 gene sequence. In some embodiments, where the target sequence is a gene, a knockin is generated resulting in the exogenous nucleic acid sequence being in operable linkage with any upstream and/or downstream regulatory elements controlling expression of the target gene. In some embodiments, the knockin is generated resulting in the exogenous nucleic acid sequence not being in operable linkage with any upstream and/or downstream regulatory elements controlling expression of the target gene.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the disclosure can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Modified Immune Cells

The present disclosure provides modified immune cells or precursors thereof (e.g., T cells) comprising a modification in an endogenous gene locus encoding SOX and/or ID3. In certain embodiments, the cell comprising a nucleic acid capable of downregulating gene expression of endogenous SOX and/or ID3. In certain embodiments, the cell comprises a nucleic acid capable of overexpressing endogeneous SOX and/or ID3. In certain embodiments, the cell further comprises an exogenous TCR and/or CAR.

In one aspect, the disclosure provides a modified immune cell or precursor thereof (e.g., T cell) comprising a CRISPR-mediated modification in an endogenous gene locus encoding SOX and/or ID3 that is capable of downregulating gene expression of endogenous SOX and/or ID3, and an exogenous CAR. In another aspect, the disclosure provides a modified immune cell or precursor thereof (e.g., T cell) comprising a CRISPR-mediated modification in an endogenous gene locus encoding SOX and/or ID3 that is capable of downregulating gene expression of endogenous SOX and/or ID3, and an exogenous TCR.

In another aspect, the disclosure provides a modified immune cell or precursor thereof (e.g., T cell) comprising a modification in an endogenous gene locus encoding SOX and/or ID3 that is capable of overexpression of endogenous SOX and/or ID3, and an exogenous CAR. In another aspect, the disclosure provides a modified immune cell or precursor thereof (e.g., T cell) comprising a modification in an endogenous gene locus encoding SOX and/or ID3 that is capable of overexpression of endogenous SOX and/or ID3, and an exogenous TCR.

The TCR and/or CAR comprises affinity for an antigen on a target cell. Accordingly, such modified cells possess the specificity directed by the TCR and/or CAR that is expressed therein. For example, a modified cell of the present disclosure comprising a NY-ESO-1 TCR possesses specificity for NY-ESO-1 on a target cell.

The present disclosure provides gene edited modified cells. In some embodiments, a modified cell (e.g., a modified cell comprising an exogenous TCR and/or CAR) of the present disclosure is genetically edited to disrupt the expression of an endogenous gene locus encoding SOX and/or ID3. In some embodiments, the gene-edited immune cells (e.g., T cells) have a downregulation, reduction, deletion, elimination, knockout or disruption in expression of the endogeneous SOX and/or ID3. In some embodiments, the gene-edited immune cells (e.g., T cells) have an overexpression of endogeneous SOX and/or ID3.

Immunotherapies using CAR (chimeric antigen receptor) T cells and TCR redirected T cells have shown various efficacies in the treatment of cancer patients. One of the major problems limiting their effects is that T cells are exhausted after persistent stimulation by tumor cells. Exhausted T cells have reduced effector functions such as production of cytokines and cytotoxicity against tumor cells, and they express higher levels of checkpoint inhibitory molecules, such as PD-1 and CTLA-4. PD-1 and CTLA-4 antibodies have been used clinically to treat multiple types of cancers.

In some embodiments, the modified cell of the present disclosure is genetically edited to disrupt the expression of an additional endogeneous gene. For example, the cell may be further edited to disrupt an endogenous PDCD1 gene product (e.g. Programmed Death 1 receptor; PD-1). Disrupting the expression of endogenous PD-1 may create “checkpoint” resistant modified cells, resulting in increased tumor control. Checkpoint resistant modified cells may also be created by disrupting the expression of, for example, without limitation, the Adenosine A2A receptor (A2AR), B7-H3 (CD276), B7-H4 (VTCN1), the B and T Lymphocyte Attenuator protein (BTLA/CD272), CD96, the Cytotoxic T-Lymphocyte Associated protein 4 (CTLA-4/CD152), Indoleamine 2,3-dioxygenase (IDO), the Killer-cell Immunoglobulin-like Receptor (KIR), the Lymphocyte Activation Gene-3 (LAG3), the T cell immunoreceptor with Ig and ITIM domains (TIGIT), T-cell Immunoglobulin domain and Mucin domain 3 (TIM-3), or the V-domain Ig suppressor of T cell activation (VISTA).

Various gene editing technologies are known to those skilled in the art. Gene editing technologies include, without limitation, homing endonucleases, zinc-finger nucleases (ZFNs), transcription activator-like effector (TALE) nucleases (TALENs), and clustered regularly interspaced short palindromic repeats (CRISPR)-associated protein 9 (Cas9). Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. The Cas9 nuclease is targeted to DNA sequences complementary to the targeting sequence within the single guide RNA (gRNA) located immediately upstream of a compatible protospacer adjacent motif (PAM). Accordingly, one of skill in the art would be able to select the appropriate gene editing technology for the present disclosure.

In some aspects, the disruption is carried out by gene editing using an RNA-guided nuclease such as a CRISPR-Cas system, such as CRISPR-Cas9 system, specific for the gene (e.g., SOX and/or ID3) being disrupted. In some embodiments, an agent containing a Cas9 and a guide RNA (gRNA) containing a targeting domain, which targets a region of the genetic locus, is introduced into the cell. In some embodiments, the agent is or comprises a ribonucleoprotein (RNP) complex of a Cas9 polypeptide and a gRNA (Cas9/gRNA RNP). In some embodiments, the introduction includes contacting the agent or portion thereof with the cells in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap.

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and TCR T cells. The CRISPR/Cas9 system can simultaneously target multiple genomic loci by co-expressing a single Cas9 protein with two or more gRNAs, making this system suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The REC I domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, a pAd5F35-CRISPR vector. In other embodiments, the Cas expression vector induces expression of Cas9 endonuclease. Other endonucleases may also be used, including but not limited to, Cas12a (Cpf1), T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). Other inducible promoters known by those of skill in the art can also be used. The inducing agent can be a selective condition (e.g., exposure to an agent, for example an antibiotic) that results in induction of the inducible promoter. This results in expression of the Cas expression vector.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” or “target sequence” is the DNA sequence in a genome of a cell that is complementary to the guide sequence of the gRNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of a CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas nuclease, a crRNA, and a tracrRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In some embodiments, the CRISPR/Cas is derived from a type II CRISPR/Cas system. In other embodiments, the CRISPR/Cas system is derived from a Cas9 nuclease. Exemplary Cas9 nucleases that may be used in the present disclosure include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9).

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present disclosure. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present disclosure may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, guide RNA(s) and Cas9 can be delivered to a cell as a ribonucleoprotein (RNP) complex (e.g., a Cas9/RNA-protein complex). RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI). In some embodiments, the Cas9/RNA-protein complex is delivered into a cell by electroporation.

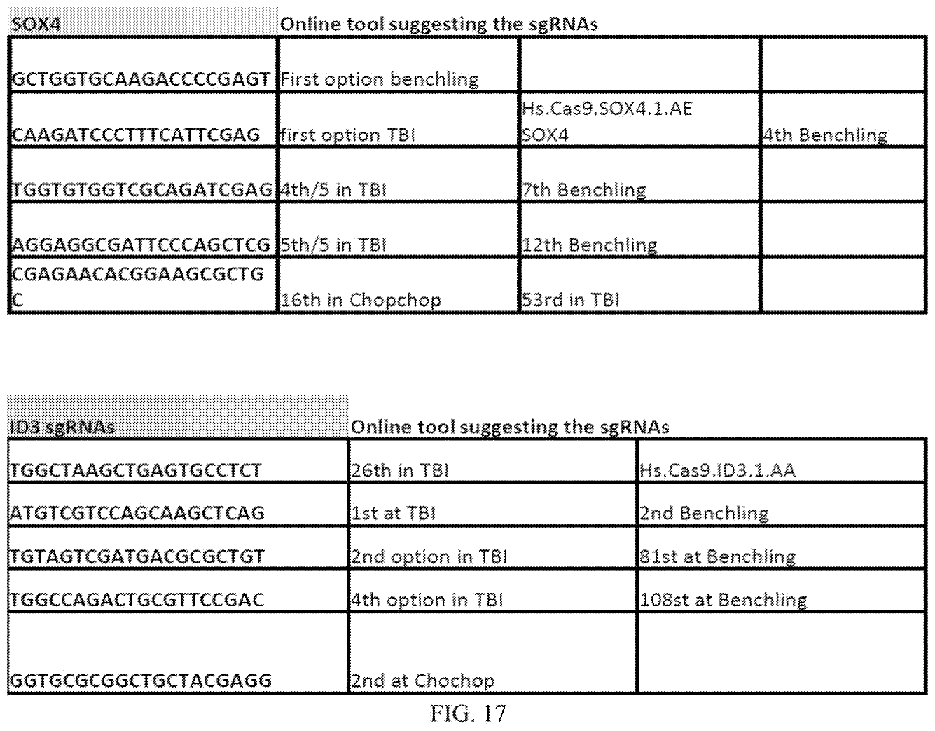

In some embodiments, a modified cell of the present disclosure is edited using CRISPR/Cas9 to disrupt an endogenous gene locus encoding SOX and/or ID3. Suitable gRNAs for use in disrupting SOX and/or ID3 are set forth herein (see FIG. 17) and include but are not limited to SEQ ID NOs: 1-5 (targeting SOX) and SEQ ID NOs: 6-10 (targeting IDs). It will be understood to those of skill in the art that guide RNA sequences may be recited with a thymidine (T) or a uridine (U) nucleotide.

Accordingly, provided in the disclosure is a modified immune cell or precursor cell thereof comprising a CRISPR-mediated modification in an endogenous gene locus encoding SOX and/or ID3, wherein the modification is capable of downregulating gene expression of endogenous SOX and/or ID3; and an exogenous T cell receptor (TCR) and/or chimeric antigen receptor (CAR) comprising affinity for an antigen on a target cell.

Non-limiting types of CRISPR-mediated modifications include a substitution, an insertion, a deletion, and an insertion/deletion (INDEL). The modification can be located in any part of the endogenous gene locus encoding SOX and/or ID3, including but not limited to an exon, a splice donor, or a splice acceptor.

In certain embodiments, the guide RNA comprises a guide sequence that is sufficiently complementary with a target sequence in the endogenous gene locus encoding SOX and/or ID3. In certain embodiments, the guide RNA comprises a guide sequence that is sufficiently complementary with a target sequence in the endogenous gene locus encoding SOX, such as, for example, a guide sequence comprising any one of the sequences set forth in SEQ ID NOs. 1-5. In certain embodiments, the guide RNA comprises a guide sequence that is sufficiently complementary with a target sequence in the endogenous gene locus encoding ID3, such as, for example, a guide sequence comprising any one of the sequences set forth in SEQ ID NOs. 6-10.

In certain embodiments, the modified cell is resistant to cell dysfunction. In certain embodiments, the modified cell is resistant to cell exhaustion. In certain embodiments, the modified cell is an autologous cell. In certain embodiments, the modified cell is a cell isolated from a human subject. In certain embodiments, the modified cell is a modified immune cell. In certain embodiments, the modified cell is a modified T cell. In certain embodiments, the modified cell is a modified T cell resistant to T cell exhaustion. In certain embodiments, the modified cell is a modified T cell resistant to T cell dysfunction.

In some aspects, the provided compositions and methods include those in which at least or greater than about 50%, 60%, 65%, 70%, 75%, 80%, 85%, 90% or 95% of immune cells in a composition of immune cells contain the desired genetic modification. For example, about 50%, 60%, 65%, 70%, 75%, 80%, 85%, 90% or 95% of immune cells in a composition of cells into which an agent (e.g. gRNA/Cas9) for knockout or genetic disruption of endogenous gene (e.g., SOX and/or ID3) was introduced contain the genetic disruption; do not express the targeted endogenous polypeptide, or do not contain a contiguous and/or functional copy of the targeted gene. In some embodiments, the methods, compositions and cells according to the present disclosure include those in which at least or greater than about 50%, 60%, 65%, 70%. 75%, 80%, 85%, 90% or 95% of cells in a composition of cells into which an agent (e.g. gRNA/Cas9) for knockout or genetic disruption of a targeted gene was introduced do not express the targeted polypeptide, such as on the surface of the immune cells. In some embodiments, at least or greater than about 50%, 60%, 65%, 70%, 75%, 80%, 85%, 90% or 95% of cells in a composition of cells into which an agent (e.g. gRNA/Cas9) for knockout or genetic disruption of the targeted gene was introduced are knocked out in both alleles, i.e. comprise a biallelic deletion, in such percentage of cells.

In some embodiments, provided are compositions and methods in which the Cas9-mediated cleavage efficiency (% indel) in or near the targeted gene (e.g. within or about within 100 base pairs, within or about within 50 base pairs, or within or about within 25 base pairs or within or about within 10 base pairs upstream or downstream of the cut site) is at least or greater than about 50%, 60%, 65%, 70%, 75%, 80%, 85%, 90% or 95% in cells of a composition of cells into which an agent (e.g. gRNA/Cas9) for knockout or genetic disruption of a targeted gene has been introduced.

In some embodiments, the provided cells, compositions and methods results in a reduction or disruption of signals delivered via the endogenous in at least or greater than about 50%, 60%, 65%, 70%, 75%, 80%, 85%, 90% or 95% of cells in a composition of cells into which an agent (e.g. gRNA/Cas9) for knockout or genetic disruption of a targeted gene was introduced.

In some embodiments, compositions according to the provided disclosure that comprise cells engineered with a recombinant receptor and comprise the reduction, deletion, elimination, knockout or disruption in expression of an endogenous gene (e.g. genetic disruption of SOX and/or ID3) retain the functional property or activities of the receptor compared to the receptor expressed in engineered cells of a corresponding or reference composition comprising the receptor but do not comprise the genetic disruption of a gene or express the polypeptide when assessed under the same conditions. In some embodiments, the engineered cells of the provided compositions retain a functional property or activity compared to a corresponding or reference composition comprising engineered cells in which such are engineered with the recombinant receptor but do not comprise the genetic disruption or express the targeted polypeptide when assessed under the same conditions. In some embodiments, the cells retain cytotoxicity, proliferation, survival or cytokine secretion compared to such a corresponding or reference composition.

In some embodiments, the immune cells in the composition retain a phenotype of the immune cell or cells compared to the phenotype of cells in a corresponding or reference composition when assessed under the same conditions. In some embodiments, cells in the composition include naive cells, effector memory cells, central memory cells, stem central memory cells, effector memory cells, and long-lived effector memory cells. In some embodiments, the percentage of T cells, or T cells expressing the recombinant receptor (e.g. TCR and/or CAR), and comprising the genetic disruption of a targeted gene (e.g., SOX and/or ID3) exhibit a non-activated, long-lived memory or central memory phenotype that is the same or substantially the same as a corresponding or reference population or composition of cells engineered with the recombinant receptor but not containing the genetic disruption. In some embodiments, such property, activity or phenotype can be measured in an in vitro assay, such as by incubation of the cells in the presence of an antigen targeted by the TCR and/or CAR, a cell expressing the antigen and/or an antigen-receptor activating substance. In some embodiments, any of the assessed activities, properties or phenotypes can be assessed at various days following electroporation or other introduction of the agent, such as after or up to 3, 4, 5, 6, 7 days. In some embodiments, such activity, property or phenotype is retained by at least 80%, 85%, 90%, 95% or 100% of the cells in the composition compared to the activity of a corresponding composition containing cells engineered with the recombinant receptor but not comprising the genetic disruption of the targeted gene when assessed under the same conditions.

As used herein, reference to a “corresponding composition” or a “corresponding population of immune cells” (also called a “reference composition” or a “reference population of cells”) refers to immune cells (e.g., T cells) obtained, isolated, generated, produced and/or incubated under the same or substantially the same conditions, except that the immune cells or population of immune cells were not introduced with the agent. In some aspects, except for not containing introduction of the agent, such immune cells are treated identically or substantially identically as immune cells that have been introduced with the agent, such that any one or more conditions that can influence the activity or properties of the cell, including the upregulation or expression of the inhibitory molecule, is not varied or not substantially varied between the cells other than the introduction of the agent.

Methods and techniques for assessing the expression and/or levels of T cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, antigen receptor (e.g. TCR and/or CAR)-expressing cells can be detected by flow cytometry or other immunoaffinity based method for expression of a marker unique to such cells, and then such cells can be co-stained for another T cell surface marker or markers.

In some embodiments, the cells, compositions and methods provide for the deletion, knockout, disruption, or reduction in expression of the target gene in immune cells (e.g. T cells) to be adoptively transferred (such as cells engineered to express an exogenous TCR and/or CAR). In some embodiments, the methods are performed ex vivo on primary cells, such as primary immune cells (e.g. T cells) from a subject. In some aspects, methods of producing or generating such genetically engineered T cells include introducing into a population of cells containing immune cells (e.g. T cells) one or more nucleic acid encoding a recombinant receptor (e.g. exogenous TCR and/or CAR) and an agent or agents that is capable of disrupting, a gene that encode the endogenous receptor to be targeted. As used herein, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The population of cells containing T cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the nucleic acid encoding a genetically engineered antigen receptor and the step of introducing the agent (e.g. Cas9/gRNA RNP) can occur simultaneously or sequentially in any order. In some embodiments, subsequent to introduction of the exogenous receptor and one or more gene editing agents (e.g. Cas9/gRNA RNP), the cells are cultured or incubated under conditions to stimulate expansion and/or proliferation of cells.

Thus, provided are cells, compositions and methods that enhance immune cell, such as T cell, function in adoptive cell therapy, including those offering improved efficacy, such as by increasing activity and potency of administered genetically engineered cells, while maintaining persistence or exposure to the transferred cells over time. In some embodiments, the genetically engineered cells, exhibit increased expansion and/or persistence when administered in vivo to a subject, as compared to certain available methods. In some embodiments, the provided immune cells exhibit increased persistence when administered in vivo to a subject. In some embodiments, the persistence of genetically engineered immune cells, in the subject upon administration is greater as compared to that which would be achieved by alternative methods, such as those involving administration of cells genetically engineered by methods in which T cells were not introduced with an agent that reduces expression of or disrupts a gene encoding an endogenous receptor. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the exogenous receptor (e.g., TCR and/or CAR) in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the exogenous receptor (e.g. exogenous TCR and/or CAR) can be used to distinguish the administered cells from endogenous cells in a subject.

Provided herein is a modified immune cell or precursor thereof (e.g., T cells) comprising a modification in an endogenous gene locus encoding SOX and/or ID3 and further comprising a CAR. Any CAR known in the art and/or disclosed herein can be included in the cell. In certain embodiments, the CAR comprises an antigen binding domain that binds human mesothelin. In certain embodiments, the CAR comprises an antigen binding domain that binds a tumor associated antigen (TAA). In certain embodiments, the CAR comprises an antigen binding domain that binds human CD19. In certain embodiments, the CAR comprises an antigen binding domain that binds GD2 (e.g., human GD2). In certain embodiments, the CAR comprises an antigen binding domain that binds HER2 (e.g., human HER2). In certain embodiments, the CAR comprises an antigen binding domain comprising a high affinity anti-HER2 scFv. In certain embodiments, the CAR comprises an antigen binding domain comprising a low affinity anti-HER2 scFv. In certain embodiments, the CAR comprises an antigen binding domain that binds TnMuc1 (e.g., human TnMuc1). In certain embodiments, the CAR comprises an antigen binding domain that binds PSMA (e.g., human PSMA). In certain embodiments, the CAR comprises an antigen binding domain that binds EGFR (e.g., EGFRvIII; e.g., human EGFRvIII). In certain embodiments, the CAR comprises an antigen binding domain that binds Fibroblast Activation Protein (FAP) (e.g., human FAP). In certain embodiments, the CAR comprises an antigen binding domain that comprises an amino acid sequence at least 85%, 90%, 95%, 96%, 97%, 98%, 99%, or 100% identical to any one of SEQ ID NOs: 13, 26, 39, 41, 43, 45, 47, 49, or 51; or is encoded by a nucleotide sequence at least 80%, 85%, 90%, 95%, 96%, 97%, 98%, 99%, or 100% identical to any one of SEQ ID NOs: 20, 32, 40, 42, 44, 46, 48, 50, or 52. In certain embodiments, the CAR comprises an amino acid sequence at least 85%, 90%, 95%, 96%, 97%, 98%, 99%, or 100% identical to any one of SEQ ID NOs: 11, 25, or 37; or is encoded by a nucleotide sequence at least 80%, 85%, 90%, 95%, 96%, 97%, 98%, 99%, or 100% identical to any one of SEQ ID NOs: 18, 30, or 38.

### C. Chimeric Antigen Receptors

The present disclosure provides compositions and methods for modified immune cells or precursors thereof, e.g., modified T cells, comprising a chimeric antigen receptor (CAR). Thus, in some embodiments, the immune cell has been genetically modified to express the CAR. CARs of the present disclosure comprise an antigen binding domain, a transmembrane domain, and an intracellular domain. In certain embodiments, the modified immune cell or precursor thereof comprises a modification in an endogenous gene locus encoding SOX and/or ID3 that is capable of downregulating gene expression of the endogenous SOX and/or ID3, and an exogeneous CAR comprising affinity for an antigen on a target cell. In certain embodiments, the modified immune cell or precursor thereof comprises a modification in an endogenous gene locus encoding SOX and/or ID3 that is capable of overexpression of the endogenous SOX and/or ID3, and an exogeneous CAR comprising affinity for an antigen on a target cell.

The antigen binding domain may be operably linked to another domain of the CAR, such as the transmembrane domain or the intracellular domain, both described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present disclosure. A subject CAR of the present disclosure may also include a hinge domain as described herein. A subject CAR of the present disclosure may also include a spacer domain as described herein. In some embodiments, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen on a target cell. The target antigen may include any type of protein, or epitope thereof, associated with the target cell. For example, the CAR may comprise affinity to a target antigen on a target cell that indicates a particular disease state of the target cell.

In certain embodiments, the target cell antigen is a tumor associated antigen (TAA). Examples of tumor associated antigens (TAAs), include but are not limited to, differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS. In a preferred embodiment, the antigen binding domain of the CAR targets an antigen that includes but is not limited to CD19, CD20, CD22, ROR1, Mesothelin, CD33/IL3Ra, c-Met, PSMA, PSCA, Glycolipid F77, EGFRvIII, GD-2, MY-ESO-1 TCR, MAGE A3 TCR, and the like.

Depending on the desired antigen to be targeted, the CAR of the disclosure can be engineered to include the appropriate antigen binding domain that is specific to the desired antigen target. For example, if CD19 is the desired antigen that is to be targeted, an antibody for CD19 can be used as the antigen bind moiety for incorporation into the CAR of the disclosure.

In one embodiment, the target cell antigen is a prostate stem cell antigen (PSCA). As such, in one embodiment, a CAR of the present disclosure has affinity for PSCA on a target cell. In one embodiment, the target cell antigen is CD19. As such, in one embodiment, a CAR of the present disclosure has affinity for CD19 on a target cell. This should not be construed as limiting in any way, as a CAR having affinity for any target antigen is suitable for use in a composition or method of the present disclosure.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. In one embodiment, a CAR of the present disclosure having affinity for CD19 on a target cell may comprise a CD19 binding domain.

In some embodiments, a CAR of the present disclosure may have affinity for one or more target antigens on one or more target cells. In some embodiments, a CAR may have affinity for one or more target antigens on a target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some embodiments, the antigen binding domain is selected from the group consisting of an antibody, an antigen binding fragment (Fab), and a single-chain variable fragment (scFv). In some embodiments, a PSCA binding domain of the present disclosure is selected from the group consisting of a PSCA-specific antibody, a PSCA-specific Fab, and a PSCA-specific scFv. In one embodiment, a PSCA binding domain is a PSCA-specific antibody. In one embodiment, a PSCA binding domain is a PSCA-specific Fab. In one embodiment, a PSCA binding domain is a PSCA-specific scFv. In some embodiments, a PSCA binding domain of the present disclosure is selected from the group consisting of a CD19-specific antibody, a CD19-specific Fab, and a CD19-specific scFv. In one embodiment, a CD19 binding domain is a CD19-specific antibody. In one embodiment, a CD19 binding domain is a CD19-specific Fab. In one embodiment, a CD19 binding domain is a CD19-specific scFv.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen binding domain portion comprises a mammalian antibody or a fragment thereof. The choice of antigen binding domain may depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., PSCA binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration for use in the present disclosure.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO:53), (GGGS)ₙ (SEQ ID NO:54), and (GGGGS)ₙ (SEQ ID NO:55), where n represents an integer of at least 1. Exemplary linker sequences can comprise amino acid sequences including, without limitation, GGSG (SEQ ID NO:56), GGSGG (SEQ ID NO:57), GSGSG (SEQ ID NO:58), GSGGG (SEQ ID NO:59), GGGSG (SEQ ID NO:60), GSSSG (SEQ ID NO:61), GGGGS (SEQ ID NO:62), GGGGSGGGGSGGGGS (SEQ ID NO:63) and the like. Those of skill in the art would be able to select the appropriate linker sequence for use in the present disclosure. In one embodiment, an antigen binding domain of the present disclosure comprises a heavy chain variable region (VH) and a light chain variable region (VL), wherein the VH and VL is separated by the linker sequence having the amino acid sequence GGGGSGGGGSGGGGS (SEQ ID NO:63), which may be encoded by the nucleic acid sequence GGTGGCGGTGGCTCGGGCGGTGGTGGGTCGGGTGGCGGCGGATCT (SEQ ID NO:64).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 August 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof.

In certain embodiments, the CAR comprises an antigen binding domain that binds human mesothelin. In certain embodiments, the CAR comprises an antigen binding domain that binds human CD19. In certain embodiments, the CAR comprises an antigen binding domain that binds GD2 (e.g., human GD2). In certain embodiments, the CAR comprises an antigen binding domain that binds HER2 (e.g., human HER2). In certain embodiments, the CAR comprises an antigen binding domain comprising a high affinity anti-HER2 scFv. In certain embodiments, the CAR comprises an antigen binding domain comprising a low affinity anti-HER2 scFv. In certain embodiments, the CAR comprises an antigen binding domain that binds TnMuc1 (e.g., human TnMuc1). In certain embodiments, the CAR comprises an antigen binding domain that binds PSMA (e.g., human PSMA). In certain embodiments, the CAR comprises an antigen binding domain that binds EGFR (e.g., EGFRvIII; e.g., human EGFRvIII). In certain embodiments, the CAR comprises an antigen binding domain that binds Fibroblast Activation Protein (FAP) (e.g., human FAP).

In certain embodiments, the antigen binding domain comprises an amino acid sequence at least 85%, 90%, 95%, 96%, 97%, 98%, 99%, or 100% identical to any one of SEQ ID NOs: 13, 26, 39, 41, 43, 45, 47, 49, or 51; or is encoded by a nucleotide sequence at least 80%, 85%, 90%, 95%, 96%, 97%, 98%, 99%, or 100% identical to any one of SEQ ID NOs: 20, 32, 40, 42, 44, 46, 48, 50, or 52.

### Transmembrane Domain

CARs of the present disclosure may comprise a transmembrane domain that connects the antigen binding domain of the CAR to the intracellular domain of the CAR. The transmembrane domain of a subject CAR is a region that is capable of spanning the plasma membrane of a cell (e.g., an immune cell or precursor thereof). In some embodiments, the transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this disclosure include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a subject CAR.

In some embodiments, the transmembrane domain further comprises a hinge region. A subject CAR of the present disclosure may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than 20 amino acids (e.g., 30, 40, 50, 60 or more amino acids).

For example, hinge regions include glycine polymers (G)ₙ, glycine-serine polymers (including, for example, (GS)ₙ, (GSGGS)ₙ (SEQ ID NO:53) and (GGGS)ₙ (SEQ ID NO:54), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO:56), GGSGG (SEQ ID NO:57), GSGSG (SEQ ID NO:58), GSGGG (SEQ ID NO:59), GGGSG (SEQ ID NO:60), GSSSG (SEQ ID NO:61), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO:65); CPPC (SEQ ID NO:66); CPEPKSCDTPPPCPR (SEQ ID NO:67) (see, e.g., Glaser et al., J. Biol. Chem. (2005) 280:41494-41503); ELKTPLGDTTHT (SEQ ID NO:68); KSCDKTHTCP (SEQ ID NO:69); KCCVDCP (SEQ ID NO:70); KYGPPCP (SEQ ID NO:71); EPKSCDKTHTCPPCP (SEQ ID NO:72) (human IgG1 hinge); ERKCCVECPPCP (SEQ ID NO:73) (human IgG2 hinge); ELKTPLGDTTHTCPRCP (SEQ ID NO:74) (human IgG3 hinge); SPNMVPHAHHAQ (SEQ ID NO:75) (human IgG4 hinge); and the like.

The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:76); see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A subject CAR of the present disclosure also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the disclosure include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcyRIII, FcsRI, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 11d, ITGAE, CD103, ITGAL, CD 11a, LFA-1, ITGAM, CDlib, ITGAX, CD 11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present disclosure include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

In certain embodiments, the CAR comprises an amino acid sequence at least 85%, 90%, 95%, 96%, 97%, 98%, 99%, or 100% identical to any one of SEQ ID NOs: 11, 25, or 37; or is encoded by a nucleotide sequence at least 80%, 85%, 90%, 95%, 96%, 97%, 98%, 99%, or 100% identical to any one of SEQ ID NOs: 18, 30, or 38.

Exemplary CAR Sequences:

Human anti-MSLN CAR AA sequence (SEQ ID NO: 11):
CD8 Leader (SEQ ID NO: 12)
MALPVTALLLPLALLLHAARP
M5 scFv (SEQ ID NO: 13)
QVQLVQSGAEVEKPGASVKVSCKASGYTFTDYYMHWVRQAPGQGLEWMGWINPNSG
GTNYAQKFQGRVTMTRDTSISTAYMELSRLRSDDTAVYYCASGWDFDYWGQGTLVTV
SSGGGGSGGGGSGGGGSGGGGSDIVMTQSPSSLSASVGDRVTITCRASQSIRYYLSWYQ
QKPGKAPKLLIYTASILQNGVPSRFSGSGSGTDFTLTISSLQPEDFATYYCLQTYTTPDFG
PGTKVEIK
CD8H (SEQ ID NO: 14)
TTTPAPRPPTPAPTIASQPLSLRPEACRPAAGGAVHTRGLDFACD
CD8TM (SEQ ID NO: 15)
IYIWAPLAGTCGVLLLSLVITLYC
4-1BBICD (SEQ ID NO: 16)
KRGRKKLLYIFKQPFMRPVQTTQEEDGCSCRFPEEEEGGCEL
CD3z (SEQ ID NO: 17)
RVKFSRSADAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
Human anti-MSLN CAR NT sequence (SEQ ID NO: 18):
CD8 Leader (SEQ ID NO: 19)
ATGGCCCTCCCTGTCACCGCCCTGCTGCTTCCGCTGGCTCTTCTGCTCCACGCCGCTC
GGCCC
M5 scFv (SEQ ID NO: 20)
CAAGTCCAACTCGTTCAATCAGGCGCAGAAGTCGAAAAGCCCGGAGCATCAGTCAA
AGTCTCTTGCAAGGCTTCCGGCTACACCTTCACGGACTACTACATGCACTGGGTGCG
CCAGGCTCCAGGCCAGGGACTGGAGTGGATGGGATGGATCAACCCGAATTCCGGGG
GAACTAACTACGCCCAGAAGTTTCAGGGCCGGGTGACTATGACTCGCGATACCTCG
ATCTCGACTGCGTACATGGAGCTCAGCCGCCTCCGGTCGGACGATACCGCCGTGTAC
TATTGTGCGTCGGGATGGGACTTCGACTACTGGGGGCAGGGCACTCTGGTCACTGTG
TCAAGCGGAGGAGGTGGATCAGGTGGAGGTGGAAGCGGGGGAGGAGGTTCCGGCG
GCGGAGGATCAGATATCGTGATGACGCAATCGCCTTCCTCGTTGTCCGCATCCGTGG
GAGACAGGGTGACCATTACTTGCAGAGCGTCCCAGTCCATTCGGTACTACCTGTCGT
GGTACCAGCAGAAGCCGGGGAAAGCCCCAAAACTGCTTATCTATACTGCCTCGATC
CTCCAAAACGGCGTGCCATCAAGATTCAGCGGTTCGGGCAGCGGGACCGACTTTAC
CCTGACTATCAGCAGCCTGCAGCCGGAAGATTTCGCCACGTACTACTGCCTGCAAAC
CTACACCACCCCGGACTTCGGACCTGGAACCAAGGTGGAGATCAAG
CD8H (SEQ ID NO: 21)
ACCACTACCCCAGCACCGAGGCCACCCACCCCGGCTCCTACCATCGCCTCCCAGCCT
CTGTCCCTGCGTCCGGAGGCATGTAGACCCGCAGCTGGTGGGGCCGTGCATACCCG
GGGTCTTGACTTCGCCTGCGAT
CD8TM (SEQ ID NO: 22)
ATCTACATTTGGGCCCCTCTGGCTGGTACTTGCGGGGTCCTGCTGCTTTCACTCGTGA
TCACTCTTTACTGT
4-1BBICD (SEQ ID NO: 23)
AAGCGCGGTCGGAAGAAGCTGCTGTACATCTTTAAGCAACCCTTCATGAGGCCTGTG
CAGACTACTCAAGAGGAGGACGGCTGTTCATGCCGGTTCCCAGAGGAGGAGGAAGG
CGGCTGCGAACTG
CD3z (SEQ ID NO: 24)
CGCGTGAAATTCAGCCGCAGCGCAGATGCTCCAGCCTACAAGCAGGGGCAGAACCA
GCTCTACAACGAACTCAATCTTGGTCGGAGAGAGGAGTACGACGTGCTGGACAAGC
GGAGAGGACGGGACCCAGAAATGGGCGGGAAGCCGCGCAGAAAGAATCCCCAAGA
GGGCCTGTACAACGAGCTCCAAAAGGATAAGATGGCAGAAGCCTATAGCGAGATTG
GTATGAAAGGGGAACGCAGAAGAGGCAAAGGCCACGACGGACTGTACCAGGGACT
CAGCACCGCCACCAAGGACACCTATGACGCTCTTCACATGCAGGCCCTGCCGCCTCGG
Human anti-CD19 CAR AA sequence (CD28 co-stimulation) (SEQ ID NO: 25):
CD8 leader (SEQ ID NO: 12)
MALPVTALLLPLALLLHAARP
hCD19 scFv (SEQ ID NO: 26)
EIVMTQSPATLSLSPGERATLSCRASQDISKYLNWYQQKPGQAPRLLIYHTSRLHSGIPAR
FSGSGSGTDYTLTISSLQPEDFAVYFCQQGNTLPYTFGQGTKLEIKGGGGSGGGGSGGG
GSQVQLQESGPGLVKPSETLSLTCTVSGVSLPDYGVSWIRQPPGKGLEWIGVIWGSETTY
YQSSLKSRVTISKDNSKNQVSLKLSSVTAADTAVYYCAKHYYYGGSYAMDYWGQGTL
VTVSS
CD8H (SEQ ID NO: 14)
TTTPAPRPPTPAPTIASQPLSLRPEACRPAAGGAVHTRGLDFACD
CD28TM (SEQ ID NO: 27)
FWVLVVVGGVLACYSLLVTVAFIIFWV
CD28ICD (SEQ ID NO: 28)
RSKRSRLLHSDYMNMTPRRPGPTRKHYQPYAPPRDFAAYRS
CD3z (SEQ ID NO: 29)
RVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
Human anti-CD19 CAR NT sequence (CD28 co-stimulation) (SEQ ID NO: 30):
CD8 leader (SEQ ID NO: 31)
ATGGCCTTACCAGTGACCGCCTTGCTCCTGCCGCTGGCCTTGCTGCTCCACGCCGCC
AGGCCG
hCD19 scFv (SEQ ID NO: 32)
GAAATTGTGATGACCCAGTCACCCGCCACTCTTAGCCTTTCACCCGGTGAGCGCGCA
ACCCTGTCTTGCAGAGCCTCCCAAGACATCTCAAAATACCTTAATTGGTATCAACAG
AAGCCCGGACAGGCTCCTCGCCTTCTGATCTACCACACCAGCCGGCTCCATTCTGGA
ATCCCTGCCAGGTTCAGCGGTAGCGGATCTGGGACCGACTACACCCTCACTATCAGC
TCACTGCAGCCAGAGGACTTCGCTGTCTATTTCTGTCAGCAAGGGAACACCCTGCCC
TACACCTTTGGACAGGGCACCAAGCTCGAGATTAAAGGTGGAGGTGGCAGCGGAGG
AGGTGGGTCCGGCGGTGGAGGAAGCCAGGTCCAACTCCAAGAAAGCGGACCGGGT
CTTGTGAAGCCATCAGAAACTCTTTCACTGACTTGTACTGTGAGCGGAGTGTCTCTC
CCCGATTACGGGGTGTCTTGGATCAGACAGCCACCGGGGAAGGGTCTGGAATGGAT
TGGAGTGATTTGGGGCTCTGAGACTACTTACTACCAATCATCCCTCAAGTCACGCGT
CACCATCTCAAAGGACAACTCTAAGAATCAGGTGTCACTGAAACTGTCATCTGTGAC
CGCAGCCGACACCGCCGTGTACTATTGCGCTAAGCATTACTATTATGGCGGGAGCTA
CGCAATGGATTACTGGGGACAGGGTACTCTGGTCACCGTGTCCAGC
CD8H (SEQ ID NO: 33)
ACCACGACGCCAGCGCCGCGACCACCAACACCGGCGCCCACCATCGCGTCGCAGCC
CCTGTCCCTGCGCCCAGAGGCGTGCCGGCCAGCGGCGGGGGGCGCAGTGCACACGA
GGGGGCTGGACTTCGCCTGTGAT
CD28TM (SEQ ID NO: 34)
TTTTGGGTGCTGGTGGTGGTTGGTGGAGTCCTGGCTTGCTATAGCTTGCTAGTAACA
GTGGCCTTTATTATTTTCTGGGTG
CD28ICD (SEQ ID NO: 35)
AGGAGTAAGAGGAGCAGGCTCCTGCACAGTGACTACATGAACATGACTCCCCGCCG
CCCCGGGCCCACCCGCAAGCATTACCAGCCCTATGCCCCACCACGCGACTTCGCAGC
CTATCGCTCC
CD3z (SEQ ID NO: 36)
AGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCGC
Human anti-CD19 CAR AA sequence (4-1BB co-stimulation) (SEQ ID NO: 37):
CD8 leader (SEQ ID NO: 12)
MALPVTALLLPLALLLHAARP
hCD19 scFv (SEQ ID NO: 26)
EIVMTQSPATLSLSPGERATLSCRASQDISKYLNWYQQKPGQAPRLLIYHTSRLHSGIPAR
FSGSGSGTDYTLTISSLQPEDFAVYFCQQGNTLPYTFGQGTKLEIKGGGGSGGGGSGGG
GSQVQLQESGPGLVKPSETLSLTCTVSGVSLPDYGVSWIRQPPGKGLEWIGVIWGSETTY
YQSSLKSRVTISKDNSKNQVSLKLSSVTAADTAVYYCAKHYYYGGSYAMDYWGQGTL
VTVSS
CD8H (SEQ ID NO: 14)
TTTPAPRPPTPAPTIASQPLSLRPEACRPAAGGAVHTRGLDFACD
CD8TM (SEQ ID NO: 15)
IYIWAPLAGTCGVLLLSLVITLYC
4-1BBICD (SEQ ID NO: 16)
KRGRKKLLYIFKQPFMRPVQTTQEEDGCSCRFPEEEEGGCEL
CD3z (SEQ ID NO: 17)
RVKFSRSADAPAYKQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
Human anti-CD19 CAR NT sequence (4-1BB co-stimulation) (SEQ ID NO: 38):
CD8 leader (SEQ ID NO: 19)
ATGGCCCTCCCTGTCACCGCCCTGCTGCTTCCGCTGGCTCTTCTGCTCCACGCCGCTC
GGCCC
hCD19 scFv (SEQ ID NO: 32)
GAAATTGTGATGACCCAGTCACCCGCCACTCTTAGCCTTTCACCCGGTGAGCGCGCA
ACCCTGTCTTGCAGAGCCTCCCAAGACATCTCAAAATACCTTAATTGGTATCAACAG
AAGCCCGGACAGGCTCCTCGCCTTCTGATCTACCACACCAGCCGGCTCCATTCTGGA
ATCCCTGCCAGGTTCAGCGGTAGCGGATCTGGGACCGACTACACCCTCACTATCAGC
TCACTGCAGCCAGAGGACTTCGCTGTCTATTTCTGTCAGCAAGGGAACACCCTGCCC
TACACCTTTGGACAGGGCACCAAGCTCGAGATTAAAGGTGGAGGTGGCAGCGGAGG
AGGTGGGTCCGGCGGTGGAGGAAGCCAGGTCCAACTCCAAGAAAGCGGACCGGGT
CTTGTGAAGCCATCAGAAACTCTTTCACTGACTTGTACTGTGAGCGGAGTGTCTCTC
CCCGATTACGGGGTGTCTTGGATCAGACAGCCACCGGGGAAGGGTCTGGAATGGAT
TGGAGTGATTTGGGGCTCTGAGACTACTTACTACCAATCATCCCTCAAGTCACGCGT
CACCATCTCAAAGGACAACTCTAAGAATCAGGTGTCACTGAAACTGTCATCTGTGAC
CGCAGCCGACACCGCCGTGTACTATTGCGCTAAGCATTACTATTATGGCGGGAGCTA
CGCAATGGATTACTGGGGACAGGGTACTCTGGTCACCGTGTCCAGC
CD8H (SEQ ID NO: 21)
ACCACTACCCCAGCACCGAGGCCACCCACCCCGGCTCCTACCATCGCCTCCCAGCCT
CTGTCCCTGCGTCCGGAGGCATGTAGACCCGCAGCTGGTGGGGCCGTGCATACCCG
GGGTCTTGACTTCGCCTGCGAT
CD8TM (SEQ ID NO: 22)
ATCTACATTTGGGCCCCTCTGGCTGGTACTTGCGGGGTCCTGCTGCTTTCACTCGTGA
TCACTCTTTACTGT
4-1BBICD (SEQ ID NO: 23)
AAGCGCGGTCGGAAGAAGCTGCTGTACATCTTTAAGCAACCCTTCATGAGGCCTGTG
CAGACTACTCAAGAGGAGGACGGCTGTTCATGCCGGTTCCCAGAGGAGGAGGAAGG
CGGCTGCGAACTG
CD3z (SEQ ID NO: 24)
CGCGTGAAATTCAGCCGCAGCGCAGATGCTCCAGCCTACAAGCAGGGGCAGAACCA
GCTCTACAACGAACTCAATCTTGGTCGGAGAGAGGAGTACGACGTGCTGGACAAGC
GGAGAGGACGGGACCCAGAAATGGGCGGGAAGCCGCGCAGAAAGAATCCCCAAGA
GGGCCTGTACAACGAGCTCCAAAAGGATAAGATGGCAGAAGCCTATAGCGAGATTG
GTATGAAAGGGGAACGCAGAAGAGGCAAAGGCCACGACGGACTGTACCAGGGACT
CAGCACCGCCACCAAGGACACCTATGACGCTCTTCACATGCAGGCCCTGCCGCCTCGG
Anti-GD2 scFv amino acid sequence (SEQ ID NO: 39)
GSDVVMTQTPLSLPVSLGDQASISCRSSQSLVHRNGNTYLHWYLQKPGQSPKLLIHKVS
NRFSGVPDRFSGSGSGTDFTLKISRVEAEDLGVYFCSQSTHVPPLTFGAGTKLELKGGGG
SGGGGSGGGGSGGGGSEVQLLQSGPELEKPSASVMISCKASGSSFTGYNMNWVRQNIG
KSLEWIGAIDPYYGGTSYNQKFKGRATLTVDKSSSTAYMHLKSLTSEDSVYYCVSGME
YWGQGTSVTVSSSG
Anti-GD2 scFv nucleotide sequence (SEQ ID NO: 40)
GGATCCGATGTTGTGATGACCCAAACTCCACTCTCCCTGCCTGTCAGTCTTGGAGAT
CAAGCCTCCATCTCTTGCAGATCTAGTCAGAGTCTTGTACACCGTAACGGAAACACC
TATTTACATTGGTACCTGCAGAAGCCAGGCCAGTCTCCAAAGCTCCTGATTCACAAA
GTTTCCAACCGATTTTCTGGGGTCCCAGACAGGTTCAGTGGCAGTGGATCAGGGACA
GATTTCACACTCAAGATCAGCAGAGTGGAGGCTGAGGATCTGGGAGTTTATTTCTGT
TCTCAAAGTACACACGTTCCTCCGCTCACGTTCGGTGCTGGGACCAAGCTGGAGCTG
AAAGGAGGTGGCGGGTCAGGGGGTGGCGGAAGCGGAGGCGGCGGTTCAGGCGGAG
GAGGCTCGGAGGTGCAGCTTCTGCAGTCTGGACCTGAGCTGGAGAAGCCTTCCGCTT
CAGTGATGATATCCTGCAAGGCTTCTGGTTCCTCCTTCACTGGCTACAACATGAACT
GGGTGAGGCAGAATATTGGAAAGAGCCTTGAATGGATTGGAGCTATTGATCCTTACT
ACGGTGGAACTAGCTACAACCAGAAGTTCAAGGGCAGGGCCACATTGACTGTAGAC
AAATCGTCCAGCACAGCCTACATGCACCTCAAGAGCCTGACATCTGAGGACTCTGTC
TATTACTGTGTAAGCGGAATGGAGTACTGGGGTCAAGGAACCTCAGTCACCGTCTCC
TCATCCGGA
Anti-HER2 scFv (high affinity) amino acid sequence (SEQ ID NO: 41)
GDIQMTQSPSSLSASVGDRVTITCRASQDVNTAVAWYQQKPGKAPKLLIYSASFLYSGV
PSRFSGSRSGTDFTLTISSLQPEDFATYYCQQHYTTPPTFGQGTKVEIKRTGSTSGSGKPG
SGEGSEVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDVW
GQGTLVTVSS
Anti-HER2 scFv (high affinity) nucleotide sequence (SEQ ID NO: 42)
GGAGATATCCAGATGACCCAGTCCCCGAGCTCCCTGTCCGCCTCTGTGGGCGATAGG
GTCACCATCACCTGCCGTGCCAGTCAGGATGTGAATACTGCTGTAGCCTGGTATCAA
CAGAAACCAGGAAAAGCTCCGAAACTACTGATTTACTCGGCATCCTTCCTTTATTCT
GGAGTCCCTTCTCGCTTCTCTGGATCTAGATCTGGGACGGATTTCACTCTGACCATCA
GCAGTCTGCAGCCGGAAGACTTCGCAACTTATTACTGTCAGCAACATTATACTACTC
CTCCCACGTTCGGACAGGGTACCAAGGTGGAGATCAAACGCACTGGGTCTACATCT
GGATCTGGGAAGCCGGGTTCTGGTGAGGGTTCTGAGGTTCAGCTGGTGGAGTCTGGC
GGTGGCCTGGTGCAGCCAGGGGGCTCACTCCGTTTGTCCTGTGCAGCTTCTGGCTTC
AACATTAAAGACACCTATATACACTGGGTGCGTCAGGCCCCGGGTAAGGGCCTGGA
ATGGGTTGCAAGGATTTATCCTACGAATGGTTATACTAGATATGCCGATAGCGTCAA
GGGCCGTTTCACTATAAGCGCAGACACATCCAAAAACACAGCCTACCTGCAGATGA
ACAGCCTGCGTGCTGAGGACACTGCCGTCTATTATTGTTCTAGATGGGGAGGGGACG
GCTTCTATGCTATGGACGTGTGGGGTCAAGGAACCCTGGTCACCGTCTCCTCG
Anti-HER2 scFv (low affinity) amino acid sequence (SEQ ID NO: 43)
GDIQMTQSPSSLSASVGDRVTITCRASQDVNTAVAWYQQKPGKAPKLLIYSASFLESGV
PSRFSGSRSGTDFTLTISSLQPEDFATYYCQQHYTTPPTFGQGTKVEIKRTGSTSGSGKPG
SGEGSEVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFVAMDVW
GQGTLVTVSS
Anti-HER2 scFv (low affinity) nucleotide sequence (SEQ ID NO: 44)
GGAGATATCCAGATGACCCAGTCCCCGAGCTCCCTGTCCGCCTCTGTGGGCGATAGG
GTCACCATCACCTGCCGTGCCAGTCAGGATGTGAATACTGCTGTAGCCTGGTATCAA
CAGAAACCAGGAAAAGCTCCGAAACTACTGATTTACTCGGCATCCTTCCTTGAGTCT
GGAGTCCCTTCTCGCTTCTCTGGATCTAGATCTGGGACGGATTTCACTCTGACCATCA
GCAGTCTGCAGCCGGAAGACTTCGCAACTTATTACTGTCAGCAACATTATACTACTC
CTCCCACGTTCGGACAGGGTACCAAGGTGGAGATCAAACGCACTGGGTCTACATCT
GGATCTGGGAAGCCGGGTTCTGGTGAGGGTTCTGAGGTTCAGCTGGTGGAGTCTGGC
GGTGGCCTGGTGCAGCCAGGGGGCTCACTCCGTTTGTCCTGTGCAGCTTCTGGCTTC
AACATTAAAGACACCTATATACACTGGGTGCGTCAGGCCCCGGGTAAGGGCCTGGA
ATGGGTTGCAAGGATTTATCCTACGAATGGTTATACTAGATATGCCGATAGCGTCAA
GGGCCGTTTCACTATAAGCGCAGACACATCCAAAAACACAGCCTACCTGCAGATGA
ACAGCCTGCGTGCTGAGGACACTGCCGTCTATTATTGTTCTAGATGGGGAGGGGACG
GCTTCGTTGCTATGGACGTGTGGGGTCAAGGAACCCTGGTCACCGTCTCCTCG
Anti-TnMuc1 scFv amino acid sequence (SEQ ID NO: 45)
QVQLQQSDAELVKPGSSVKISCKASGYTFTDHAIHWVKQKPEQGLEWIGHFSPGNTDIK
YNDKFKGKATLTVDRSSSTAYMQLNSLTSEDSAVYFCKTSTFFFDYWGQGTTLTVSSG
GGGSGGGGSGGGGSELVMTQSPSSLTVTAGEKVTMICKSSQSLLNSGDQKNYLTWYQQ
KPGQPPKLLIFWASTRESGVPDRFTGSGSGTDFTLTISSVQAEDLAVYYCQNDYSYPLTF
GAGTKLELK
Anti-TnMuc1 scFv nucleotide sequence (SEQ ID NO: 46)
CAGGTGCAGCTGCAGCAGTCTGATGCCGAGCTCGTGAAGCCTGGCAGCAGCGTGAA
GATCAGCTGCAAGGCCAGCGGCTACACCTTCACCGACCACGCCATCCACTGGGTCA
AGCAGAAGCCTGAGCAGGGCCTGGAGTGGATCGGCCACTTCAGCCCCGGCAACACC
GACATCAAGTACAACGACAAGTTCAAGGGCAAGGCCACCCTGACCGTGGACAGAAG
CAGCAGCACCGCCTACATGCAGCTGAACAGCCTGACCAGCGAGGACAGCGCCGTGT
ACTTCTGCAAGACCAGCACCTTCTTTTTCGACTACTGGGGCCAGGGCACAACCCTGA
CAGTGTCTAGCGGAGGCGGAGGATCTGGCGGCGGAGGAAGTGGCGGAGGGGGATC
TGAACTCGTGATGACCCAGAGCCCCAGCTCTCTGACAGTGACAGCCGGCGAGAAAG
TGACCATGATCTGCAAGTCCTCCCAGAGCCTGCTGAACTCCGGCGACCAGAAGAAC
TACCTGACCTGGTATCAGCAGAAACCCGGCCAGCCCCCCAAGCTGCTGATCTTTTGG
GCCAGCACCCGGGAAAGCGGCGTGCCCGATAGATTCACAGGCAGCGGCTCCGGCAC
CGACTTTACCCTGACCATCAGCTCCGTGCAGGCCGAGGACCTGGCCGTGTATTACTG
CCAGAACGACTACAGCTACCCCCTGACCTTCGGAGCCGGCACCAAGCTGGAACTGA
AG
Anti-PSMA scFv amino acid sequence (SEQ ID NO: 47)
GSDIVMTQSHKFMSTSVGDRVSIICKASQDVGTAVDWYQQKPGQSPKLLIYWASTRHT
GVPDRFTGSGSGTDFTLTITNVQSEDLADYFCQQYNSYPLTFGAGTMLDLKGGGGSGG
GGSSGGGSEVQLQQSGPELVKPGTSVRISCKTSGYTFTEYTIHWVKQSHGKSLEWIGNIN
PNNGGTTYNQKFEDKATLTVDKSSSTAYMELRSLTSEDSAVYYCAAGWNFDYWGQGT
TLTVSSASSG
Anti-PSMA scFv nucleotide sequence (SEQ ID NO: 48)
GGATCTGACATTGTGATGACCCAGTCTCACAAATTCATGTCCACATCAGTAGGAGAC
AGGGTCAGCATCATCTGTAAGGCCAGTCAAGATGTGGGTACTGCTGTAGACTGGTAT
CAACAGAAACCAGGACAATCTCCTAAACTACTGATTTATTGGGCATCCACTCGGCAC
ACTGGAGTCCCTGATCGCTTCACAGGCAGTGGATCTGGGACAGACTTCACTCTCACC
ATTACTAACGTTCAGTCTGAAGACTTGGCAGATTATTTCTGTCAGCAATATAACAGC
TATCCTCTCACGTTCGGTGCTGGGACCATGCTGGACCTGAAAGGAGGCGGAGGATCT
GGCGGCGGAGGAAGTTCTGGCGGAGGCAGCGAGGTGCAGCTGCAGCAGAGCGGAC
CCGAGCTCGTGAAGCCTGGAACAAGCGTGCGGATCAGCTGCAAGACCAGCGGCTAC
ACCTTCACCGAGTACACCATCCACTGGGTCAAGCAGTCCCACGGCAAGAGCCTGGA
GTGGATCGGCAATATCAACCCCAACAACGGCGGCACCACCTACAACCAGAAGTTCG
AGGACAAGGCCACCCTGACCGTGGACAAGAGCAGCAGCACCGCCTACATGGAACTG
CGGAGCCTGACCAGCGAGGACAGCGCCGTGTACTATTGTGCCGCCGGTTGGAACTT
CGACTACTGGGGCCAGGGCACAACCCTGACAGTGTCTAGCGCTAGCTCCGGA
Anti-EGFRvIII sc Fv amino acid sequence (SEQ ID NO: 49)
EIQLVQSGAEVKKPGESLRISCKGSGFNIEDYYIHWVRQMPGKGLEWMGRIDPENDETK
YGPIFQGHVTISADTSINTVYLQWSSLKASDTAMYYCAFRGGVYWGQGTTVTVSSGGG
GSGGGGSGGGGSGGGGSDVVMTQSPDSLAVSLGERATINCKSSQSLLDSDGKTYLNWL
QQKPGQPPKRLISLVSKLDSGVPDRFSGSGSGTDFTLTISSLQAEDVAVYYCWQGTHFPG
TFGGGTKVEIK
Anti-EGFRvIII scFv nucleotide sequence (SEQ ID NO: 50)
GAGATTCAGCTCGTGCAATCGGGAGCGGAAGTCAAGAAGCCAGGAGAGTCCTTGCG
GATCTCATGCAAGGGTAGCGGCTTTAACATCGAGGATTACTACATCCACTGGGTGAG
GCAGATGCCGGGGAAGGGACTCGAATGGATGGGACGGATCGACCCAGAAAACGAC
GAAACTAAGTACGGTCCGATCTTCCAAGGCCATGTGACTATTAGCGCCGATACTTCA
ATCAATACCGTGTATCTGCAATGGTCCTCATTGAAAGCCTCAGATACCGCGATGTAC
TACTGTGCTTTCAGAGGAGGGGTCTACTGGGGACAGGGAACTACCGTGACTGTCTCG
TCCGGCGGAGGCGGGTCAGGAGGTGGCGGCAGCGGAGGAGGAGGGTCCGGCGGAG
GTGGGTCCGACGTCGTGATGACCCAGAGCCCTGACAGCCTGGCAGTGAGCCTGGGC
GAAAGAGCTACCATTAACTGCAAATCGTCGCAGAGCCTGCTGGACTCGGACGGAAA
AACGTACCTCAATTGGCTGCAGCAAAAGCCTGGCCAGCCACCGAAGCGCCTTATCTC
ACTGGTGTCGAAGCTGGATTCGGGAGTGCCCGATCGCTTCTCCGGCTCGGGATCGGG
TACTGACTTCACCCTCACTATCTCCTCGCTTCAAGCAGAGGACGTGGCCGTCTACTA
CTGCTGGCAGGGAACCCACTTTCCGGGAACCTTCGGCGGAGGGACGAAAGTGGAGA
TCAAG
Anti-FAP scFv amino acid sequence (SEQ ID NO: 51)
QIVLTQSPALMSASPGEKVTMTCTASSSVSYMYWYQQKPRSSPKPWIFLTSNLASGVPA
RFSGRGSGTSFSLTISSMEAEDAATYYCQQWSGYPPITFGSGTKLEIKGGGGSGGGGSGG
GGSQVQLQQPGAELVKPGASVKLSCKASGYTITSYSLHWVKQRPGQGLEWIGEINPANG
DHNFSEKFEIKATLTVDSSSNTAFMQLSRLTSEDSAVYYCTRLDDSRFHWYFDVWGAG
TTVTVSS
Anti-FAP scFv nucleotide sequence (SEQ ID NO: 52)
CAAATTGTTCTCACCCAGTCTCCAGCGCTCATGTCTGCTTCTCCAGGGGAGAAGGTC
ACCATGACCTGCACTGCCAGCTCAAGTGTTAGTTACATGTACTGGTACCAGCAGAAG
CCACGATCCTCCCCCAAACCCTGGATTTTTCTCACCTCCAACCTGGCTTCTGGAGTCC
CTGCTCGCTTCAGTGGCCGTGGGTCTGGGACCTCTTTCTCTCTCACAATCAGCAGCAT
GGAGGCTGAAGATGCTGCCACTTATTACTGCCAGCAGTGGAGTGGTTACCCACCCAT
CACATTCGGCTCGGGGACAAAGTTGGAAATAAAAGGTGGAGGTGGCAGCGGAGGA
GGTGGGTCCGGCGGTGGAGGAAGCCAGGTCCAACTGCAGCAGCCTGGGGCTGAACT
GGTAAAGCCTGGGGCTTCAGTGAAGTTGTCCTGCAAGGCGTCTGGCTACACCATCAC
CAGCTACTCTCTGCACTGGGTGAAGCAGAGGCCTGGACAAGGCCTTGAGTGGATTG
GAGAGATTAATCCTGCCAATGGTGATCATAACTTCAGTGAGAAGTTCGAGATCAAG
GCCACACTGACTGTAGACAGCTCCTCCAACACAGCATTCATGCAACTCAGCAGGCTG
ACATCTGAGGACTCTGCGGTCTATTACTGTACAAGATTGGACGATAGTAGGTTCCAC
TGGTACTTCGATGTCTGGGGCGCAGGGACCACGGTCACCGTCTCCTCA

### D. T Cell Receptors

The present disclosure provides compositions and methods for modified immune cells or precursors thereof (e.g., modified T cells) comprising an exogenous T cell receptor (TCR). Thus, in some embodiments, the cell has been altered to contain specific T cell receptor (TCR) genes (e.g., a nucleic acid encoding an alpha/beta TCR). TCRs or antigen-binding portions thereof include those that recognize a peptide epitope or T cell epitope of a target polypeptide, such as an antigen of a tumor, viral or autoimmune protein. In certain embodiments, the TCR has binding specificity for a tumor associated antigen, e.g., human NY-ESO-1. In certain embodiments, the immune cell or precursor thereof comprises a modification in the endogenous gene locus encoding SOX and/or ID3 that is capable of downregulating gene expression of the endogenous SOX and/or ID3, and an exogeneous TCR comprising affinity for an antigen on a target cell. In certain embodiments, the immune cell or precursor thereof comprises a modification in the endogenous gene locus encoding SOX and/or ID3 that is capable of overexpression of the endogenous SOX and/or ID3, and an exogeneous TCR comprising affinity for an antigen on a target cell.

A TCR is a disulfide-linked heterodimeric protein comprised of six different membrane bound chains that participate in the activation of T cells in response to an antigen. There exists alpha/beta TCRs and gamma/delta TCRs. An alpha/beta TCR comprises a TCR alpha chain and a TCR beta chain. T cells expressing a TCR comprising a TCR alpha chain and a TCR beta chain are commonly referred to as alpha/beta T cells. Gamma/delta TCRs comprise a TCR gamma chain and a TCR delta chain. T cells expressing a TCR comprising a TCR gamma chain and a TCR delta chain are commonly referred to as gamma/delta T cells. A TCR of the present disclosure is a TCR comprising a TCR alpha chain and a TCR beta chain.

The TCR alpha chain and the TCR beta chain are each comprised of two extracellular domains, a variable region and a constant region. The TCR alpha chain variable region and the TCR beta chain variable region are required for the affinity of a TCR to a target antigen. Each variable region comprises three hypervariable or complementarity-determining regions (CDRs) which provide for binding to a target antigen. The constant region of the TCR alpha chain and the constant region of the TCR beta chain are proximal to the cell membrane. A TCR further comprises a transmembrane region and a short cytoplasmic tail. CD3 molecules are assembled together with the TCR heterodimer. CD3 molecules comprise a characteristic sequence motif for tyrosine phosphorylation, known as immunoreceptor tyrosine-based activation motifs (ITAMs). Proximal signaling events are mediated through the CD3 molecules, and accordingly, TCR-CD3 complex interaction plays an important role in mediating cell recognition events.

Stimulation of TCR is triggered by major histocompatibility complex molecules (MHCs) on antigen presenting cells that present antigen peptides to T cells and interact with TCRs to induce a series of intracellular signaling cascades. Engagement of the TCR initiates both positive and negative signaling cascades that result in cellular proliferation, cytokine production, and/or activation-induced cell death.

A TCR of the present disclosure can be a wild-type TCR, a high affinity TCR, and/or a chimeric TCR. A high affinity TCR may be the result of modifications to a wild-type TCR that confers a higher affinity for a target antigen compared to the wild-type TCR. A high affinity TCR may be an affinity-matured TCR. Methods for modifying TCRs and/or the affinity-maturation of TCRs are known to those of skill in the art. Techniques for engineering and expressing TCRs include, but are not limited to, the production of TCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840).

In some embodiments, the exogenous TCR is a full TCR or an antigen-binding portion or antigen-binding fragment thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al, Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the j-chain can contain a further hypervariable region (CDR4 or HVR4), which generally is involved in superantigen binding and not antigen recognition (Kotb (1995) Clinical Microbiology Reviews, 8:411-426).

In some embodiments, a TCR contains a variable alpha domain (V_{α}) and/or a variable beta domain (V_{β}) or antigen-binding fragments thereof. In some embodiments, the α-chain and/or β-chain of a TCR also can contain a constant domain, a transmembrane domain and/or a short cytoplasmic tail (see, e.g., Janeway et al., Immunobiology: The Immune System in Health and Disease, 3 Ed., Current Biology Publications, p. 4:33, 1997). In some embodiments, the α chain constant domain is encoded by the TRAC gene (IMGT nomenclature) or is a variant thereof. In some embodiments, the 3 chain constant region is encoded by TRBC1 or TRBC2 genes (IMGT nomenclature) or is a variant thereof. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

It is within the level of a skilled artisan to determine or identify the various domains or regions of a TCR. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). Using this system, the CDR1 sequences within a TCR Va chain and/or Vβ chain correspond to the amino acids present between residue numbers 27-38, inclusive, the CDR2 sequences within a TCR Va chain and/or Vβ chain correspond to the amino acids present between residue numbers 56-65, inclusive, and the CDR3 sequences within a TCR Va chain and/or Vβ chain correspond to the amino acids present between residue numbers 105-117, inclusive. The IMGT numbering system should not be construed as limiting in any way, as there are other numbering systems known to those of skill in the art, and it is within the level of the skilled artisan to use any of the numbering systems available to identify the various domains or regions of a TCR.

In some embodiments, the TCR may be a heterodimer of two chains α and β (or optionally γ and δ) that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, the T cells can be a cultured T cell hybridoma or clone. In some embodiments, the TCR or antigen-binding portion thereof can be synthetically generated from knowledge of the sequence of the TCR. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14: 1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments as described, the TCR can contain an introduced disulfide bond or bonds. In some embodiments, the native disulfide bonds are not present. In some embodiments, the one or more of the native cysteines (e.g. in the constant domain of the α chain and β chain) that form a native interchain disulfide bond are substituted with another residue, such as with a serine or alanine. In some embodiments, an introduced disulfide bond can be formed by mutating non-cysteine residues on the alpha and beta chains, such as in the constant domain of the α chain and β chain, to cysteine. Exemplary non-native disulfide bonds of a TCR are described in published International PCT No. WO2006/000830 and WO2006/037960. In some embodiments, cysteines can be introduced at residue Thr48 of the α chain and Ser57 of the β chain, at residue Thr45 of the α chain and Ser77 of the β chain, at residue Tyr10 of the α chain and Ser17 of the β chain, at residue Thr45 of the α chain and Asp59 of the β chain and/or at residue Ser15 of the α chain and Glu15 of the β chain. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCR chains contain a transmembrane domain. In some embodiments, the transmembrane domain is positively charged. In some cases, the TCR chain contains a cytoplasmic tail. In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3y, CD35, CD3s and CD3ζ (chains) contain one or more immunoreceptor tyrosine-based activation motif or ITAM that are involved in the signaling capacity of the TCR complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, for purposes of the provided methods, the TCR is in cell-bound form expressed on the surface of a cell. In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native interchain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane. In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR, which is a single amino acid strand containing an α chain and a β chain that is able to bind to MHC-peptide complexes. Typically, a scTCR can be generated using methods known to those of skill in the art, See e.g., International published PCT Nos. WO 96/13593, WO 96/18105, WO99/18129, WO04/033685, WO2006/037960, WO2011/044186; U.S. Pat. No. 7,569,664; and Schlueter, C. J. et al. J. Mol. Biol. 256, 859 (1996). In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment. In some embodiments, a scTCR contains a first segment constituted by an amino acid sequence corresponding to a TCR β chain variable region, a second segment constituted by an amino acid sequence corresponding to a TCR α chain variable region sequence fused to the N terminus of an amino acid sequence corresponding to a TCR α chain constant domain extracellular sequence, and a linker sequence linking the C terminus of the first segment to the N terminus of the second segment. In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment. In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence comprising an α chain extracellular constant domain sequence and transmembrane sequence, and, optionally, a linker sequence linking the C terminus of the first segment to the N terminus of the second segment. In some embodiments, for the scTCR to bind an MHC-peptide complex, the α and β chains must be paired so that the variable region sequences thereof are orientated for such binding. Various methods of promoting pairing of an α and β in a scTCR are well known in the art. In some embodiments, a linker sequence is included that links the α and β chains to form the single polypeptide strand. In some embodiments, the linker should have sufficient length to span the distance between the C terminus of the α chain and the N terminus of the β chain, or vice versa, while also ensuring that the linker length is not so long so that it blocks or reduces bonding of the scTCR to the target peptide-MHC complex. In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P-, wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, a scTCR contains a disulfide bond between residues of the single amino acid strand, which, in some cases, can promote stability of the pairing between the α and β regions of the single chain molecule (see e.g. U.S. Pat. No. 7,569,664). In some embodiments, the scTCR contains a covalent disulfide bond linking a residue of the immunoglobulin region of the constant domain of the α chain to a residue of the immunoglobulin region of the constant domain of the β chain of the single chain molecule. In some embodiments, the disulfide bond corresponds to the native disulfide bond present in a native dTCR. In some embodiments, the disulfide bond in a native TCR is not present. In some embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described above. In some cases, both a native and a non-native disulfide bond may be present.

In some embodiments, any of the TCRs, including a dTCR or scTCR, can be linked to signaling domains that yield an active TCR on the surface of a T cell. In some embodiments, the TCR is expressed on the surface of cells. In some embodiments, the TCR does contain a sequence corresponding to a transmembrane sequence. In some embodiments, the transmembrane domain can be a Ca or CP transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3. In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal.

In some embodiments, the TCR comprises affinity to a target antigen on a target cell. The target antigen may include any type of protein, or epitope thereof, associated with the target cell. For example, the TCR may comprise affinity to a target antigen on a target cell that indicates a particular disease state of the target cell. In some embodiments, the target antigen is processed and presented by MHCs.

In one embodiment, the target cell antigen is a New York esophageal-1 (NY-ESO-1) peptide. NY-ESO-1 belongs to the cancer-testis (CT) antigen group of proteins. NY-ESO-1 is a highly immunogenic antigen in vitro and is presented to T cells via the MHC. CTLs recognizing the A2 presented epitope NY-ESO₁₅₇₋₁₆₅, SLLMWITQC (SEQ ID NO:77), have been grown from the blood and lymph nodes of myeloma patients. T cell clones specific for this epitope have been shown to kill tumor cells. A high affinity TCR recognizing the NY-ESO₁₅₇₋₁₆₅ epitope may recognize HLA-A2-positive, NY-ESO-1 positive cell lines (but not to cells that lack either HLA-A2 or NY-ESO). Accordingly, a TCR of the present disclosure may be a HLA-A2-restricted NY-ESO-1 (SLLMWITQC; SEQ ID NO:77)-specific TCR. In one embodiment, an NY-ESO-1 TCR of the present disclosure is a wild-type NY-ESO-1 TCR. A wild-type NY-ESO-1 TCR may include, without limitation, the 8F NY-ESO-1 TCR (also referred to herein as “8F” or “8F TCR”), and the 1G4 NY-ESO-1 TCR (also referred to herein as “1G4” or “1G4 TCR”). In one embodiment, an NY-ESO-1 TCR of the present disclosure is an affinity enhanced 1G4 TCR, also called Ly95. 1G4 TCR and affinity enhanced 1G4 TCR is described in U.S. Pat. No. 8,143,376. This should not be construed as limiting in any way, as a TCR having affinity for any target antigen is suitable for use in a composition or method of the present disclosure.

### E. Methods of Producing Genetically Modified Immune Cells

The present disclosure provides methods for producing or generating a modified immune cell or precursor thereof (e.g., a T cell) of the disclosure for tumor immunotherapy, e.g., adoptive immunotherapy. The cells generally are engineered by introducing one or more genetically engineered nucleic acids encoding the exogenous receptors (e.g., a TCR and/or CAR). In some embodiments, the cells also are introduced, either simultaneously or sequentially with the nucleic acid encoding the exogenous receptor, with an agent (e.g. Cas9/gRNA RNP or plasmid) that is capable of disrupting or overexpressing a targeted gene (e.g., a gene encoding for SOX and/or ID3).

In certain embodiments, the disclosure provides a method for generating a modified immune cell or precursor cell thereof, comprising introducing into an immune or precursor cell a CRISPR system comprising one or more polypeptides and/or nucleic acids capable of downregulating gene expression of endogenous SOX and/or ID3; and introducing into the immune or precursor cell a nucleic acid encoding an exogenous TCR and/or CAR, wherein the exogenous TCR and/or CAR comprises affinity for an antigen on a target cell.

In yet another aspect, the disclosure provides a method for generating a modified immune cell or precursor cell thereof, comprising introducing into an immune or precursor cell one or more polypeptides and/or nucleic acids capable of overexpressing endogenous SOX and/or ID3; and introducing into the immune or precursor cell a nucleic acid encoding an exogenous TCR and/or CAR, and wherein the exogenous TCR and/or CAR comprises affinity for an antigen on a target cell.

In some embodiments, the exogenous TCR and/or CAR is introduced into a cell by an expression vector. Expression vectors comprising a nucleic acid sequence encoding a TCR and/or CAR of the present disclosure are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

In certain embodiments, the nucleic acid encoding an exogenous TCR and/or CAR is introduced into the cell via viral transduction. In certain embodiments, the viral transduction comprises contacting the immune or precursor cell with a viral vector comprising the nucleic acid encoding an exogenous TCR and/or CAR. In certain embodiments, the viral vector is an adeno-associated viral (AAV) vector. In certain embodiments, the AAV vector comprises a 5′ ITR and a 3′ITR. In certain embodiments, the AAV vector comprises a 5′ homology arm and a 3′ homology arm, wherein the 5′ and 3′ homology arms comprise complementarity to a target sequence in an endogenous gene locus encoding SOX and/or ID3. In certain embodiments, the AAV vector comprises a Woodchuck Hepatitis Virus post-transcriptional regulatory element (WPRE). In certain embodiments, the AAV vector comprises a polyadenylation (polyA) sequence. In certain embodiments, the polyA sequence is a bovine growth hormone (BGH) polyA sequence.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the TCR and/or CAR in the host cell. In some embodiments, the adenovirus genome is a 36 kb, linear, double stranded DNA, where a foreign DNA sequence (e.g., a nucleic acid encoding an exogenous TCR and/or CAR) may be inserted to substitute large pieces of adenoviral DNA in order to make the expression vector of the present disclosure (see, e.g., Danthinne and Imperiale, Gene Therapy (2000) 7(20): 1707-1714).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid (e.g., a nucleic acid encoding an exogenous TCR and/or CAR) into the viral genome at certain locations to produce a virus that is replication defective. Though the retroviral vectors are able to infect a broad variety of cell types, integration and stable expression of the TCR and/or CAR requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression, e.g., of a nucleic acid encoding a TCR and/or CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell.

The present disclosure also provides genetically engineered cells which include and stably express a TCR and/or CAR of the present disclosure. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In certain embodiments, the genetically engineered cells are autologous cells. In certain embodiments, the modified cell is resistant to T cell exhaustion. In certain embodiments, the modified cell is resistant to T cell dysfunction.

Modified cells (e.g., comprising a TCR and/or CAR) may be produced by stably transfecting host cells with an expression vector including a nucleic acid of the present disclosure. Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a TCR and/or CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). Compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots) or by assays described herein to identify agents falling within the scope of the disclosure.

In one embodiment, the nucleic acids introduced into the host cell are RNA. In another embodiment, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

In some embodiments, a nucleic acid encoding a TCR and/or CAR of the present disclosure will be RNA, e.g., in vitro synthesized RNA. Methods for in vitro synthesis of RNA are known in the art; any known method can be used to synthesize RNA comprising a sequence encoding a TCR and/or CAR. Methods for introducing RNA into a host cell are known in the art. See, e.g., Zhao et al. Cancer Res. (2010) 15: 9053. Introducing RNA comprising a nucleotide sequence encoding a TCR and/or CAR into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a TCR and/or CAR.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the disclosure is that RNA transfection is essentially transient and a vector-free. An RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the exogenous receptor (e.g., the TCR and/or CAR) and the gene editing agent (e.g. Cas9/gRNA RNP). In some embodiments, the cells (e.g. T cells) can be incubated or cultivated prior to, during or subsequent to the introduction of the nucleic acid molecule encoding the exogenous receptor, such as prior to, during or subsequent to the transduction of the cells with a viral vector (e.g. lentiviral vector) encoding the exogenous receptor. In some embodiments, the cells (e.g. T cells) can be incubated or cultivated prior to, during or subsequent to the introduction of the gene editing agent (e.g. Cas9/gRNA RNP), such as prior to, during or subsequent to contacting the cells with the agent or prior to, during or subsequent to delivering the agent into the cells, e.g. via electroporation. In some embodiments, the incubation can be both in the context of introducing the nucleic acid molecule encoding the exogenous receptor and introducing the gene editing agent, e.g. Cas9/gRNA RNP. In some embodiments, the method includes activating or stimulating cells with a stimulating or activating agent (e.g. anti-CD3/anti-CD28 antibodies) prior to introducing the nucleic acid molecule encoding the exogenous receptor and the gene editing agent, e.g. Cas9/gRNA RNP.

In some embodiments, introducing the gene editing agent, e.g. Cas9/gRNA RNP, is done after introducing the nucleic acid molecule encoding the exogenous receptor. In some embodiments, prior to the introducing of the agent, the cells are allowed to rest, e.g. by removal of any stimulating or activating agent. In some embodiments, prior to introducing the agent, the stimulating or activating agent and/or cytokines are not removed. Those of skill in the art will be able to determine the order in which each of the one or more nucleic acid sequences are introduced into the host cell.

### F. Methods of Treatment with Modified Cells

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the disclosure includes a method for adoptive cell transfer therapy comprising administering to a subject in need thereof a modified T cell of the present disclosure. In another aspect, the disclosure includes a method of treating a disease or condition in a subject comprising administering to a subject in need thereof a population of modified T cells.

Also included is a method of treating a disease or condition in a subject in need thereof comprising administering to the subject a genetically edited modified cell (e.g., genetically edited modified T cell). In certain embodiments, the method of treating a disease or condition in a subject in need thereof comprises administering to the subject a genetically edited modified cell (e.g. comprising downregulated expression or overexpression of endogenous SOX and/or ID3) comprising an exogenous TCR and/or CAR.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells of the present disclosure can be administered to an animal, preferably a mammal, even more preferably a human, to treat a cancer. In addition, the cells of the present disclosure can be used for the treatment of any condition related to a cancer, especially a cell-mediated immune response against a tumor cell(s), where it is desirable to treat or alleviate the disease. The types of cancers to be treated with the modified cells or pharmaceutical compositions of the disclosure include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells of the disclosure are used to treat a myeloma, or a condition related to myeloma. Examples of myeloma or conditions related thereto include, without limitation, light chain myeloma, non-secretory myeloma, monoclonal gamopathy of undertermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

In certain exemplary embodiments, the modified immune cells of the disclosure are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells of the disclosure are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells of the disclosure to be administered may be autologous, with respect to the subject undergoing therapy.

The administration of the cells of the disclosure may be carried out in any convenient manner known to those of skill in the art. The cells of the present disclosure may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the disclosure are injected directly into a site of inflammation in the subject, a local disease site in the subject, alymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence.

In some embodiments, the methods comprise administration of a chemotherapeutic agent. In certain embodiments, the modified cells of the disclosure (e.g., a modified cell comprising modified endogenous SOX and/or ID3 and a TCR and/or CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present disclosure.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the disclosure can be administered in dosages and routes and at times to be determined in appropriate pre-clinical and clinical experimentation and trials. Cell compositions may be administered multiple times at dosages within these ranges. Administration of the cells of the disclosure may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the disclosure provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol Blood Marrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

The modified immune cells comprising an exogenous TCR and/or CAR of the present disclosure may be used in a method of treatment as described herein. In some embodiments, the modified immune cells comprise an insertion and/or deletion in a SOX and/or ID3 gene locus that is capable of downregulating gene expression of SOX and/or ID3. In some embodiments, when SOX and/or ID3 is downregulated, the function of the immune cell comprising an exogenous TCR and/or CAR is enhanced. For example, without limitation, when downregulated, SOX and/or ID3 enhances tumor infiltration, tumor killing, and/or resistance to immunosuppression of the immune cell comprising an exogenous TCR and/or CAR. In some embodiments, when SOX and/or ID3 is downregulated, T cell exhaustion is reduced or eliminated. In some embodiments, when SOX and/or ID3 is downregulated, T cell dysfunction is reduced or eliminated.

As such, the modified immune cells comprising an exogenous TCR and/or CAR of the present disclosure when used in a method of treatment as described herein, enhances the ability of the modified immune cells in carrying out their function. Accordingly, the present disclosure provides a method for enhancing a function of a modified immune cell for use in a method of treatment as described herein.

In one aspect, the disclosure includes a method of treating cancer in a subject in need thereof, comprising administering to the subject any one of the modified immune or precursor cells disclosed herein. Yet another aspect of the disclosure includes a method of treating cancer in a subject in need thereof, comprising administering to the subject a modified immune or precursor cell generated by any one of the methods disclosed herein.

Still another aspect of the disclosure includes a method of treating a disease or disorder in a subject in need thereof, comprising administering to the subject a modified T cell comprising: a CRISPR-mediated modification in an endogenous gene locus encoding SOX and/or ID3, wherein the modification is capable of downregulating gene expression of endogenous SOX and/or ID3; and an exogenous T cell receptor (TCR) and/or chimeric antigen receptor (CAR) comprising affinity for an antigen on a target cell.

In another aspect, the disclosure includes a method of treating a disease or disorder in a subject in need thereof, comprising administering to the subject a modified T cell comprising: a modification of an endogenous gene locus encoding SOX and/or ID3, wherein the modification is capable of overexpression of endogenous SOX and/or ID3; and an exogenous T cell receptor (TCR) and/or chimeric antigen receptor (CAR) comprising affinity for an antigen on a target cell.

### G. Methods of Assessing and Treating T Cell Dysfunction

Also provided herein are methods of assessing T cell dysfunction or exhaustion, as well as methods for treating T cell dysfunction or exhaustion, in a subject in need thereof.

In one aspect, the disclosure includes a method of assessing T cell dysfunction in a subject comprising measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY. When at least 11 of the genes is upregulated, the T cell is dysfunctional.

The panel of genes can be measured by any means known to one of ordinary skill in the art, including but not limited to PCR, qPCR, microarray, sequencing, and the like. Genes can be determined to be upregulated by methods known to one of ordinary skill in the art, which include but not limited to comparison to a reference sample, comparison to a standard curve with known gene quantities, comparison to a sample taken from the subject before treatment, and the like.

In certain embodiments, the T cell or population of T cells wherein dysfunction is being measured comprise a CAR (are CAR T cells). In certain embodiments, the T cell or population of T cells wherein dysfunction is being measured comprise an engineered TCR (are used for TCR therapy). In certain embodiments, CAR and/or TCR is capable of binding a tumor associated antigen (TAA).

In another aspect, the disclosure includes a method for treating cancer in a subject in need thereof. The method comprises administering a CAR T cell therapy to the subject, then measuring a panel of genes in a sample from the subject. The panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY. When at least 11 of the genes are upregulated, the CAR T cells are deemed dysfunctional and an alternative therapy is administered.

In another aspect, the disclosure includes a method of treating cancer in a subject in need thereof comprising administering to the subject a therapy comprising a T cell comprising an engineered TCR capable of binding a tumor associated antigen (TAA), and measuring a panel of genes in a sample from the subject. The panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY. When at least 11 of the genes are upregulated, the T cells are deemed dysfunctional and an alternative therapy is administered.

In another aspect, the disclosure includes a method of treating a disease, disorder, or chronic infection in a subject in need thereof. The method comprises administering to the subject a T cell therapy, and measuring a panel of genes in a sample from the subject. The panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY. When at least 11 of the genes are upregulated, the cells are deemed dysfunctional and an alternative therapy is administered.

In certain embodiments, the chronic infection is selected from the group consisting of HIV, EBV and CMV.

An “alternative therapy” is meant to include any therapy that was not originally administered to the subject (e.g. CAR T, TCR, or T cell therapy). Alternative therapies can include but are not limited to chemotherapy, checkpoint inhibitors, cell therapy, gene therapy, and any of the treatments described elsewhere herein. The alternative treatment may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The therapies are in some embodiments suitably administered to the subject at one time or over a series of treatments.

### H. Sources of Immune Cells

In certain embodiments, a source of immune cells is obtained from a subject for ex vivo manipulation. Sources of target cells for ex vivo manipulation may also include, e.g., autologous or heterologous donor blood, cord blood, or bone marrow. For example the source of immune cells may be from the subject to be treated with the modified immune cells of the disclosure, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{hIgh}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD 14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD1 1b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### I. Expansion of Immune Cells

Whether prior to or after modification of cells (e.g. to express a TCR and/or CAR and/or modify endogenous SOX and/or ID3), the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the disclosure may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the disclosure, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the disclosure includes cryopreserving the expanded T cells. The cryopreserved T cells are thawed prior to introducing nucleic acids into the T cell.

In another embodiment, the method comprises isolating T cells and expanding the T cells. In another embodiment, the disclosure further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555, 105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### J. Pharmaceutical Compositions and Formulations

Also provided are populations of immune cells of the disclosure, compositions containing such cells and/or enriched for such cells, such as in which cells expressing the recombinant receptor make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells, or such as in which at least 50%, 60%, 70%, 80%, 90%, 91%, 92%, 93%, 94%, 95%, 96%, 97%, 98%, 99%, or more of the total cells comprise a modification in an endogenous gene locus encoding SOX and/or ID3. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

General cell culture: AsPC-1, K562 and HEK293T cells were obtained from American Type Culture Collection (ATCC). AsPC-1 cells were grown in D20 media consisting of DMEM/F12 (1:1) (Gibco, Life Technologies), 20% fetal bovine serum (FBS) and 1% penicillin/streptavidin (Gibco, Life Technologies) and K562 and HEK293T cells were cultured in R10 media consisting of RPMI-1640 (Gibco, Life Technologies) with 10% FBS, 2% HEPES (Gibco), 1% of GlutaMAX™ (Gibco), and 1% of penicillin/streptavidin. GFP-expressing cell lines were generated by lentiviral transduction for cell killing assays. All cell lines were routinely authenticated by the University of Arizona Genetics Core and tested for mycoplasma contamination (MycoAlert Mycoplasma Detection Kit, Lonza).

Lentiviral vector production: Lentiviral vector production was performed as previously described (Kutner et al., (2009) Nat Protoc 4, 495-505). Briefly, HEK293T cells were transfected with the CAR lentiviral construct and the packaging plasmids by using Lipofectamine 2000 (Invitrogen) according to manufacturer's instructions. Lentiviral supernatants were collected at 24 and 48 hours post-transfection and concentrated using high-speed ultracentrifugation. To generate the lentiviral stocks, the resulting concentrated lentivirus batches were resuspended in cold R10 media and stored at −80° C.

Transduction of CAR-redirected human T cells: Primary human CD4+T and CD8+ T cells from normal donors were provided by University of Pennsylvania Human Immunology Core. CAR T cells were generated as previously described (Carpenito et al., (2009) Proc Natl Acad Sci USA 106, 3360-3365). Briefly, CD4+ and CD8+T at 1:1 ratio at 1×10e⁶ cells/ml were activated with Dynabeads® CD3/CD28 CTS™ (Gibco, Life Technologies) at a 3:1 bead-to-cell ratio. Approximately after 24 hours, T cells were transduced at a multiplicity of infection (MOI) of 5. At day 5 beads were removed from cultures. T cell cultures were maintained at 8×10e⁵ cells/ml. Cell number and volume were monitored daily using Multisizer 4 Coulter Counter (Beckmanwaslter). Transduced T cells were cryopreserved when reached the resting state, as determined by cell size.

CAR T cell in vitro dysfunction model: AsPC-1 cells were routinely seeded in 6-well plates at 1×10e⁶ cells/well the day preceding T cell seeding. M5 CAR T cells (30-50% of transduction efficiency) were thawed and rested at 1×10e⁶ cells/ml in T75 flasks with R10 media. After 24 hours, the T cell number (CD45+EpCAM−) was calculated and 2.5×10e⁵ T cells/well were transferred to the AsPC-1 plates. After 3-4 days, the cocultures were thoroughly suspended by frequent pipetting and 300-400 μl of the cell suspension was used for T cell counting assessment and flow cytometry staining. The remaining cell suspension was spun down and the supernatant (conditioned media) was collected and filtered with a 0.45 μm filter (Corning). The cells were resuspended in media containing equal amounts of conditioned and fresh R10. The resulting T cell suspension was transferred into AsPC-1-coated plates cells (2.5×10e⁵ T cells/well) for continuous co-culture. This process was repeated for 20-35 days.

Flow cytometry and sorting: For flow cytometry and sorting assays of CAE, M5 CAR T cells were stained in fluorescence-activated cell sorting (FACS) buffer consisting of PBS (Gibco), 0.5% bovine serum albumin (BSA) (GEMINI), 2 mM EDTA (Invitrogen), and 100 μg/ml DNase (Roche). CountBright™ Absolute Counting Beads, (ThermoFisher) were used as an internal standard according to the manufacturer's instructions to calculate absolute cell counts in cell suspensions.

Antibodies specific for human CD45 (ref 304032, clone HI30), CD3 (317322, OKT3), CD4 (357412, A161A1/317440), CD56 (304608, MEM-188), EpCAM (324226/324238, 9C4), CD94 (305520, DX22), KLRB1 (339918, HP-3G10), TIGIT (372716, A15153G), TCR Va24-Jα18 (342922, 6B11), PD-1 (329928, EH12.2H7) were purchased from BioLegend. Antibodies specific for human CD8 (560179, SK1) was purchased from BD Pharmingen. Antibodies specific for human NKG2A (FAB1059P, 131411) and Methothelin (FAB32652P, 420411) were purchased from R&D Systems. Antibodies specific for human CTLA-4 (12-1529-42, 14D3) was purchased from eBioscience. Antibody specific for human NKG2C (130-103-636, REA205) was purchased from Miltenyi.

M5 CAR expression was assessed using biotinylated goat anti-human IgG F(ab′)₂ (Jackson ImmunoResearch, 109-066-006) followed by streptavidin (FITC or APC) (BioLegend) or using an anti-idiotype antibody provided by Novartis Pharmaceuticals. Live/dead staining was performed using a Live/Dead Fixable Aqua Dead Cell Stain Kit (Life Technologies following manufacturer's protocol followed by cell surface staining for 20 minutes at room temperature in the dark. Intracellular staining was performed with the Foxp3/Transcription Factor Staining Buffer set (Thermo Fisher) according to the manufacturer's instructions. Samples were acquired on an LSRII Fortessa Cytometer (BD Bioscience) and analyzed with FlowJo v10 software (FlowJo, LLC). Sorting assays were performed using a FACS Aria Cytometer (BD Bioscience).

CD56+ cell depletion: MACS Dead cell removal kit and CD56 MicroBeads (Miltenyi Biotec) were used for CD56-positive cell depletion on day 0 CAR T cell products. The CD56-depleted CAR T cell product was subjected to CAE protocol as described above and the frequency of CD56+ T cells was assessed by flow cytometry.

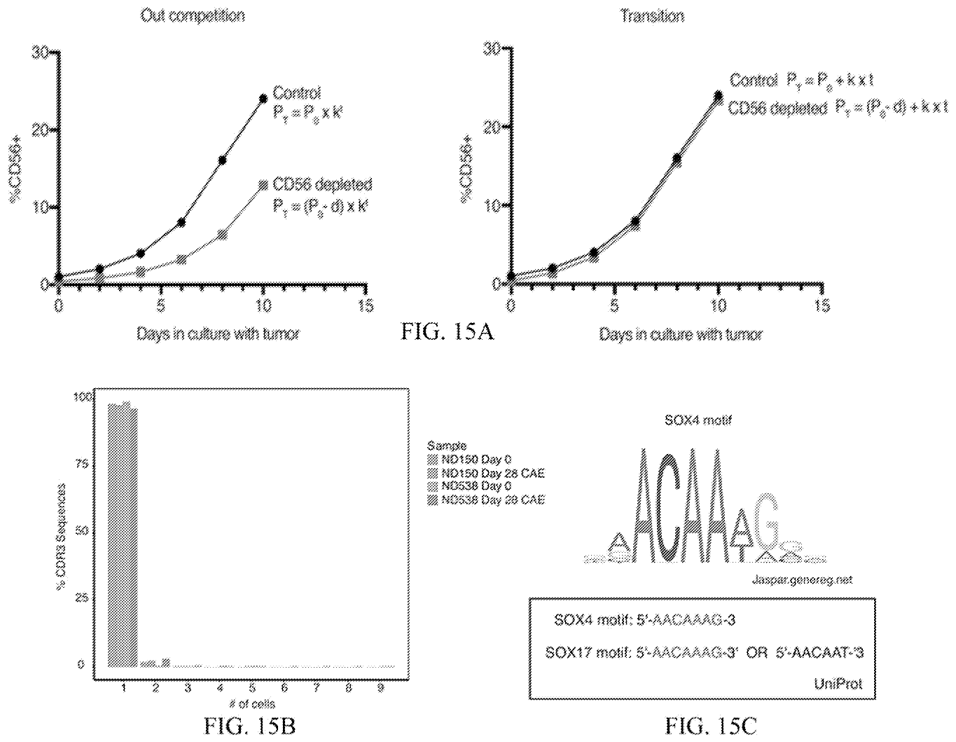

The out-competition model assumes that initial depletion of the NK-like-T cell population would result in altered kinetics of NK-like-T cell abundance over time compared to a non-depleted control group, whereas transitioning assumes similar kinetics between the control and depleted groups. As shown in FIG. 15A, in case of out-competition by the CD56-positive cell subset (left panel), the frequency of CD56 in the CD56-depleted cultures increase at a lower rate than in the controls. This growth can be expressed by the formula PT=(P0−d)×kt. On the other hand, if T cell are transitioning into NK-like-T cells, (S8A, right panel), the frequency of CD56 in the cocultures would increase at the same rate over the time, independently of the initial depletion of the CD56 at the start of the coculture, which can be expressed as PT=(P0−d)+k×t. PT: percentage CD56-positive cells at time “T”. P0: Percentage CD56-positive cells at time zero. T: time of in vitro stimulation [Days]. K: transition constant. D: percentage CD56-positive cells depleted.

Clinical trial design and research participants: Single-institution pilot safety and feasibility trial was conducted at University of Pennsylvania. This study is registered at www.clinical trial.gov as #NCT03054298. 1-3×10e⁷ M5 CAR T cells/m² were intravenously infused into patients who were diagnosed with ovarian cancer. Pleural fluid (patient 06) or peritoneal fluid (patient 01) were collected (06: day 36, 01: day 21) and surface and intracellular CAR expression was analyzed by flow cytometry. PBMCs collected from patients who received CD19CAR (CTL019) T cells to treat DLBCL (www.clinical trial.gov number, NCT02030834) and CTL019 T cell products were used for identifying NK-like CAR T cells in human. Fifty-two DLBCL patients were enrolled and 35 patients were excluded as CD56 expression was not examined. CTL019 T cell expansion in the patient's blood was analyzed by qPCR and the peak time point of expansion was selected to examine the frequency of NK-like CAR T cells. To investigate the expression of NK-related molecules on CAR T cells, cryopreserved materials from patient 13413-39 (CTL019 T cell product and PBMCs collected 27 days after CAR T infusion) were thawed and analyzed by flow cytometry.

Cytotoxicity assays: Cytotoxic killing of target cells was assessed using a real-time, impedance-based assay with xCELLigence Real-Time Cell Analyzer System (ACEA Biosciences). Briefly, 10e⁴ AsPC-1 cells were seeded to the 96-well E-plate. After 24 hours, sorted CD8+ CAE surCARpos T cells (day 28 CAE, day 0 product and CD19BBz) were added to the wells in 4:1 E:T ratio. Tumor killing was monitored every 20 minutes over 4 days.

High-throughput cytotoxicity assay using Celigo Image Cytometer (Nexcelom Bioscience) was used to investigate the effects of the resting with cytokine supplement on cytotoxicity of CAR T cells. CD8+M5CAR T cells were sorted after CAE, counted and the viability assessed using Moxi Flow System (Orflo Technologies). Part of the cell suspension was cocultured with 1.5×10e³-2×10e³ AsPC-1-GFP cells immediately after sorting in a 7:1 E:T ratio and the rest was left resting at 1.0×10e⁶ cells/ml in fresh R10 media with IL-15 supplement (20 ng/ml). After 24 h, cell viability was examined and rested T cells were cocultured with AsPC-1-GFP cells in identical conditions as the non-rested counterparts. The % lysis was calculated by direct cell counting of live fluorescent target cells. % Lysis=(1−count # of live target cells (GFP) in wells with effector cells/count # of live target cells (GFP) in wells without effector cells)×100

Cytokine production: Fifty thousand CD8+ surCARpos T cells (day 28 CAE, day 0 product and CD19BBz) were cocultured with 5×10e⁴ AsPC-1 cells or left in R10 media in 48 well plate. After 48 hours, supernatant was collected and analyzed by high-sensitivity LUMINEX assay according to manufacturer's instructions (Merck Millipore).

Quantitative real-time PCR (qPCR): Surface CAR-positive and -negative CD8+ T cells were sorted on days 4, 7 and 17 after CAE and genomic DNA was isolated from sorted cell pellets using an Arcturus™ PicoPure™ DNA Extraction Kit (Applied Biosystems). qPCR was performed in triplicate with TaqMan Gene Expression Master Mix on a 7500Fast Real-Time PCR System (Applied Biosystems) per the manufacturer's instructions. The validated primers specific to the 4-1BB and CD3ζ fusion gene and probes specific for the fusion fragment and labeled with compatible reporter dyes (FAM or VIC) were used to detect the CAR. The average plasmid copy per cell was calculated based on the factor 0.0063 ng/cell. Nine μL DNA was loaded directly for quantitation by p21 qPCR. A correction factor (CF) was not used for calculating the average % marking and copies/μg DNA as the amount of actual DNA loaded was accurately quantified by p21.

CAR re-expression assay: SurCARneg CD8+ T cells were sorted after 23 days of CAE, rested in fresh R10 media with TL-15 supplement (20 ng/ml) for 24 hours and examined for surface CAR expression by flow cytometry.

CyTOF: Mass cytometry antibodies were obtained as pre-conjugated metal-tagged antibodies from Fluidigm or prepared using the Maxpar antibody conjugation kit (Fluidigm) according to the manufacturer's protocol. Following labeling, antibodies were diluted in Candor PBS Antibody Stabilization solution (Candor Bioscience GmbH, Wangen, Germany) supplemented with 0.02% NaN3 to 0.25 mg/mL and stored long-term at 4° C. Each antibody was titrated to optimal staining concentrations using primary human PBMCs.

CAE CD8⁺ CART cells and CD8⁺ CART product were washed and resuspended 1:1 with PBS containing EDTA and 20 μM cisplatin for 2 minutes before quenching 1:1 with CSM (cell staining medium: PBS with 0.5% BSA and 0.02% NaN3) for dead cell discrimination. After washed, the cells were fixed for 10 minutes at RT using 1.6% paraformaldehyde (PFA) in PBS and frozen in CSM with 10% DMSO at −80° C. CAE CD8⁺ CART cells and CD8⁺ CART product were barcoded with distinct combinations of stable Pd isotypes in Barcode Perm Buffer (Fluidigm). Cells were washed twice with CSM, and once with PBS, and pooled into a single tube. Cells were blocked with human FcR blocking reagent (BD Bioscience) for 10 minutes at RT. Cells were then incubated with all antibodies targeting cell surface markers for 30 minutes at RT. After washed, cells were fixed with 1.6% PFA and permeabilized with Perm-S buffer (Fluidigm). Fixed/permeabilized cells were incubated with all antibodies targeting intracellular antigens for 30 minutes at room temperature. After washed with CSM, cells were incubated in 4% PFA in PBS with 191/193-iridium intercalator (Fluidigm) for 48 hours. Cells were washed in CSM, PBS, and then deionized H₂O. Cells were resuspended in deionized H₂O containing EQ four-element beads (Fluidigm) to approximately 10⁶ cells and then analyzed on Helios CyTOF system (Fluidigm) at Flow Cytometry Core, University of California, San Francisco. The acquisition data were normalized with premessa package and analyzed with cytofkit package (27662185) in R software 3.6.1 (The R foundation for Statistical Computing, Vienna, Austria).

Mouse experiments: NOD/scid/IL2rγ−/− (NSG) mice were purchased from The Jackson Laboratory and bred and housed in the vivarium at the University of Pennsylvania in pathogen-free conditions. Animal studies were approved by the University of Pennsylvania Institutional Animal Care and Use Committee.

Five million A549-A2-ESO tumor cells in 150 μl of Matrigel:PBS (1:1) solution were subcutaneously injected in the flanks of NSG mice. 2×10e⁷ human T cells were activated with anti-CD3+anti-CD28 microbeads 3:1 and subsequently transduced with 3^{rd} generation high titer lentivirus encoding for the Ly95 TCR. Transduced cells (50% of which were positive for Ly95 TCR) were intravenously injected when tumors reached a mean volume of 150 mm³. Thirty days after T cell injection, mice were sacrificed, tumors were harvested, digested and processed. The single-cell suspension obtained was then treated with Dead Cell Removal Kit (Miltenyi Biotec) following manufacturer's protocol, and CD3+ cells were positively selected by using an EasySep cell isolation kit (Stem Cell Technologies). The non-transduced CD8+ T cells from the same donor and the transduced NY-ESO-1 redirected infusion product were also subjected to the same digestion and processing protocols.

T cells from the tumor cell suspension were stained with anti-hCD8 and anti-TCRV013.1 The donor's CD8+ T cells were stained with anti-CD8 and anti-CD45RO. NY-ESO-1 T cell infusion product was stained with anti-CD8 and anti-TCRVβ13.1. All three specimens were flow sorted on the BD FACS Aria on the same day for the following populations: CD45 cells isolated from tumor digest—CD8+/TCRVβ13.1+, donor's untransduced CD8+ T cells—CD8+/CD45RO+, NY-ESO-1 T cell infusion product—CD8+/TCRVβ13.1+. Sorted samples were snap frozen, subjected to RNA extraction with Qiazol (Qiagen) and gene expression microarray analyses. For genes with multiple probes, average expression values were used to make the heatmap in R (pheatmap).

For the AsPC-1 recurrence model, NSG mice were subcutaneously injected with 2×10e6 AsPC-1 cells suspended in 200 ml Ma-trigel:PBS (1:1) into the right flank. When the mean of tumor volumes reached 300 mm3, mice were treated with 1×10e6 ND552 M5CAR+ T cells. Tumor volumes were calculated as length×width (2/2). Tumor growth was weekly assessed by caliper measurement. After primary antitumor response mice were monitored for recurrence. Mice bearing recurrent tumors were sacrificed when reached the maximum size or showed evident signs of disease, and tumors were collected. Fresh tumors were excised and digested in RPMI containing collagenase D (400 Mandl units/mL, Sigma) and DNase I (50 mg/mL, Sigma) for 15 minutes at 37 C. Enzymatic digestion was stopped with 12 mL/mL EDTA d 0.5 M, pH 8. Tumors were mechanically disrupted and filtered through a 0.7 mm cell strainer (Corning). For flow cytometry stainings, single-cell suspensions were stained with Fixable Dead Cell Dyes followed by FcR-Block treatment (Fc Receptor Blocking Solution, Biolegend) following manufacturer's recommendations. Positive NK receptor cell subsets in DO and recurrent samples were determined in sample-matched tumor and Day 0 FMO controls. Positive checkpoint receptor subsets were determined sample-matched tumor and Day 0 isotype controls. All the isotype controls were incubated at the same final concentration as their corresponding test antibody.

Single-cell RNA-seq and TCR-seq: ScRNA-seq libraries were generated using a Chromium Single-Cell 3′ Library and Gel Bead Kit (10× Genomics) using v3 for CAR T donor ND388 and v3.1 for donors ND539 and ND566 following the manufacturer's protocol. Briefly, 16,000 CD8+ T cells were sorted by flow cytometry and washed with ice cold PBS+0.04% BSA. After washing, cells were used to generate single-cell gel beads in emulsion. Following reverse transcription, gel beads in emulsion were disrupted and barcoded complementary DNA was isolated and amplified by PCR for 12 cycles. After fragmentation, end repair, and poly A tailing, samples indexes were added and amplified following the manufacturer's protocol. The final libraries were quality control checked and sequenced on an Illumina NextSeq 500 with a 150-cycle kit with parameters Read 1: 28, Read 2: 130, Index 1: 8, Index 2: 0. One sample was sequenced per flow cell. For CAR T donors ND150 and ND538, scRNA-seq libraries were generated using Chromium Single-Cell 5′ Library and Gel Bead Kit and TCR libraries were generated using Chromium Single-Cell V(D)J Reagent Kits (10× Genomics) according to the manufacturer's protocol. Followed same brief protocol as above except amplified cDNA by PCR for 13 cycles. Two uL of post amplified cDNA was used to generate TCR libraries and 50 ng of cDNA was used to generate 5′ gene expression libraries. After fragmentation, end repair and poly A tailing, sample indexes were added and amplified following manufacturer's protocol. The libraries were sequenced on an Illumina NextSeq 500 with a 150-cycle kit with parameters Read 1: 26, Read 2: 130, Index 1: 8, Index 2: 0. One RNA library and one TCR library (8:1 ratio) were pooled and sequenced on one flow cell.

Polyclonal (bulk) RNA-seq: RNA-seq libraries were made following the previously established SMARTseq2 protocol (Picelli et al., (2014) Nat Protoc 9, 171-181). Briefly, total RNA was extracted using Qiazol (Qiagen) from 300 cells for day 0, day 16 or day 28 for CD8+ T cells continuously stimulated with antigen (two sorted populations including surface CAR-positive and surface CAR-negative cells). Cells were recovered by RNA Clean and Concentrator spin columns (Zymo), followed by incubation with oligo-dT. The transcription reaction was carried out on 100 pg of cDNA for 1 min at 55° C. Libraries were uniquely barcoded (Buenrostro et al., (2013) Nat Methods 10, 1213-1218) and amplified for 14 cycles. Fragment size distribution was verified and paired-end sequencing (2×75 bp reads) was carried out on an Illumina NextSeq 500.

Paired-end data were aligned to human genome assembly GRCh37/hg19 using STAR v2.5.2a with command-line parameters --outFilterType BySJout --outFilterMultimapNmax 20 --alignSJoverhangMin 8 --alignSJDBoverhangMin 1 --outFilterMismatchNmax 999 --alignIntronMin 20 --alignIntronMax 1000000 --alignMatesGapMax 1000000. Resulting SAM files were converted to BAM format using samtools v1.1 (samtools view -bS) and BAM files were sorted by position using samtools sort. For replicate R2, several libraries were pooled after alignment to enhance coverage using samtools merge as below:
R2 Control Day 0 CAR+ T1: 4-Day0-CD8-CARpos_S7, 16-Day0-CD8-CARpos_S5
R2 Control Day 0 CAR− T1: 3-Day0-CD8-CARneg_S2, 15-Day0-CD8-CARneg_S6
R2 other CAR+ T1: 21-other-CD8-CARpos-10-24-2018_S11, 12-other-CD8-CARpos_S12, 24-other-CD8-CARpos_S10
R2 CAE CAR+ T1: 8-CAE-CD8-CARpos_S9, 20-CAE-CD8-CARpos_S12
R2 CAE CAR− T1: 7-CAE-CD8-CARneg_S6, 19-CAE-CD8-CARneg_S3

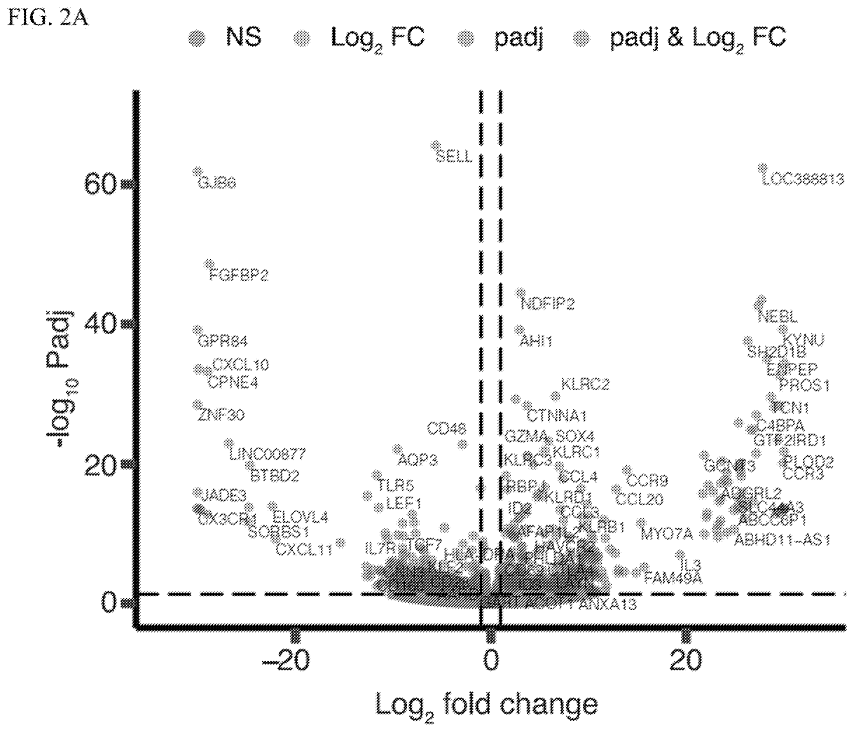

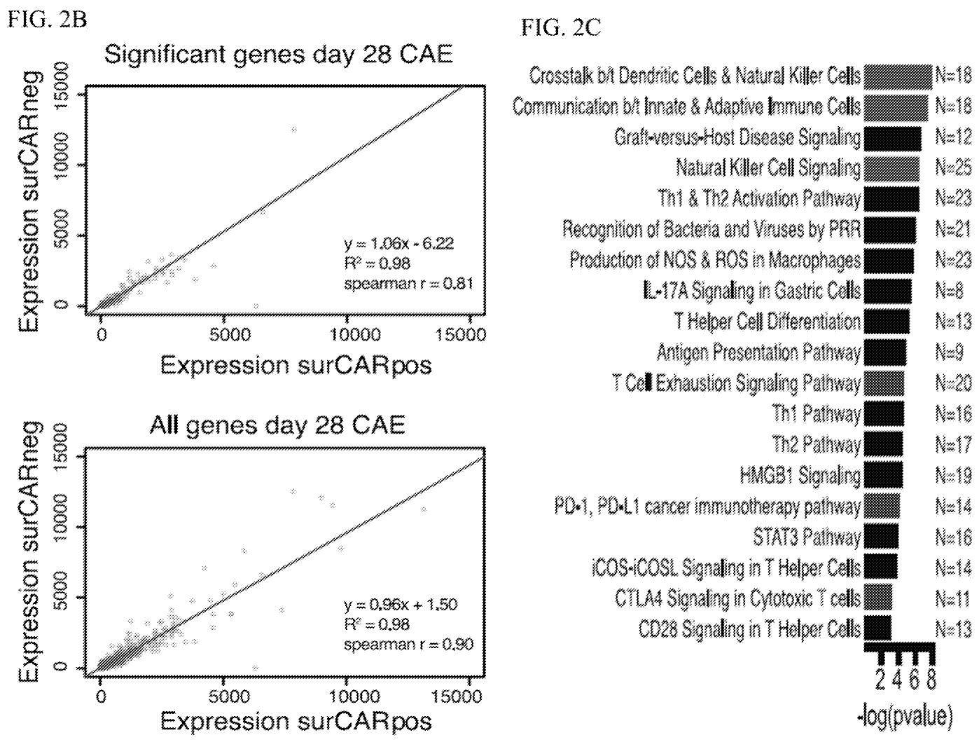

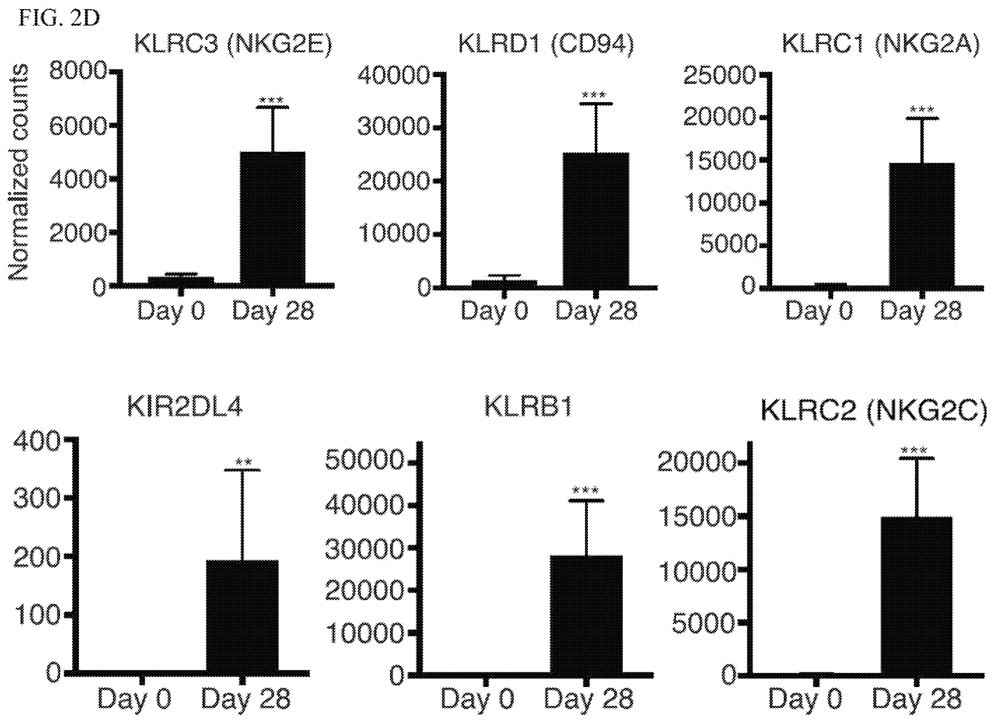

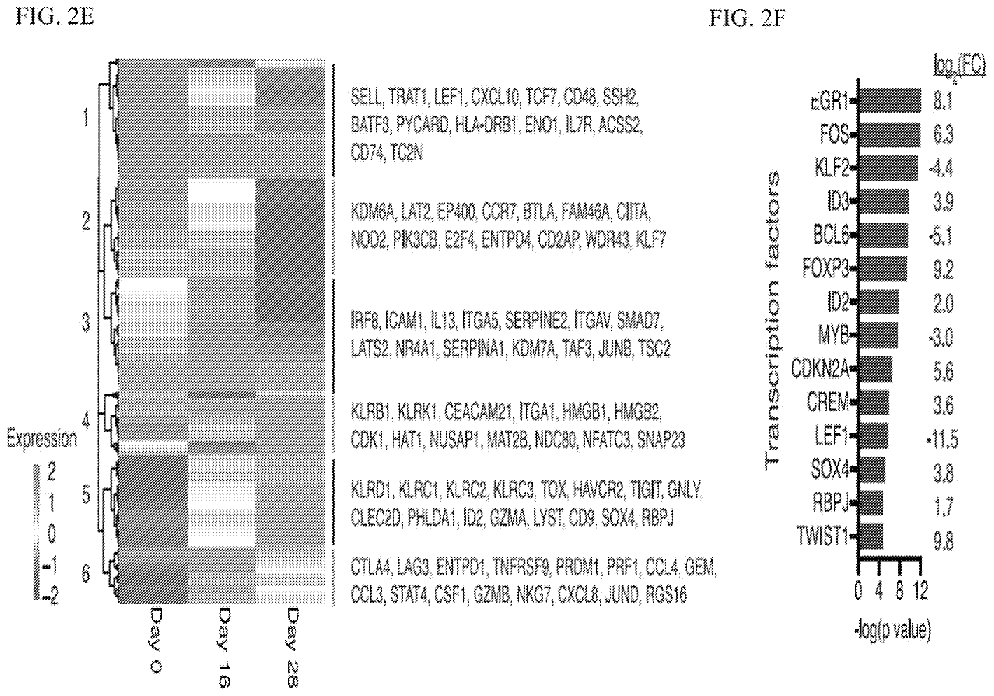

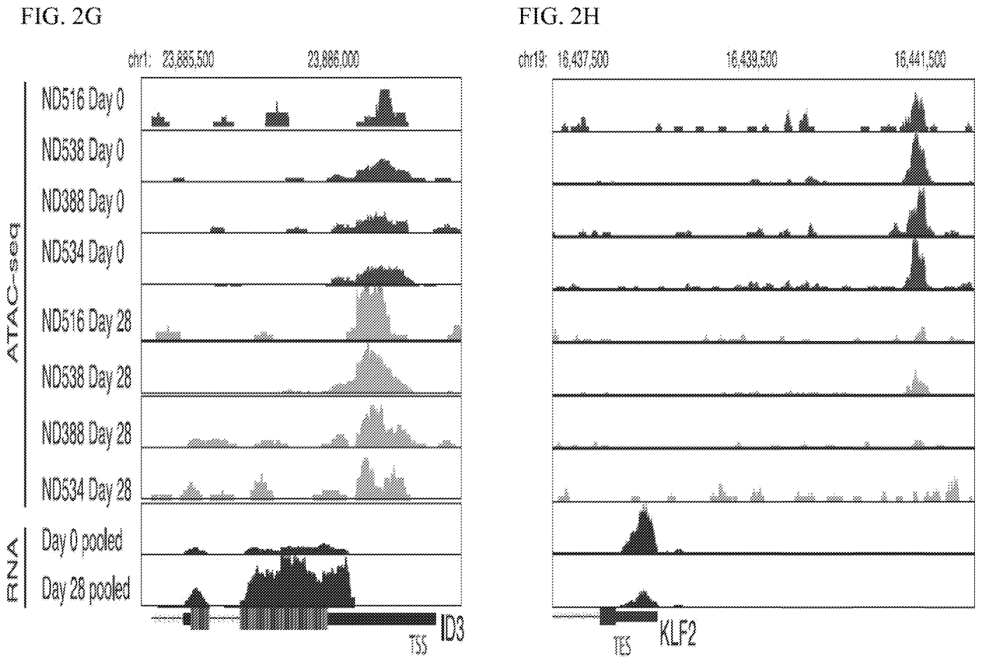

HTSeq v0.6.0 was used to count aligned tags over gene features with command-line python -m HTSeq.scripts.count -f bam -r pos -s no -t exon -i gene id BAM_FILE GTF. The GTF was constructed from RefSeq transcripts and UCSC Genome Browser's annotation of RefSeq transcript IDs to gene symbols. For the antigen exposure and time series analysis, DESeq2 was used to adjust library size and estimate significant differences at an FDR of 0.05. The Wald test was used to assess differences between control day 0 and CAE. Other samples were included to adjust dispersions and library sizes but were not used for the contrast. LRT was used to assess differences along the time course (day 0, day 16, day 28), with a full model of ˜Replicate+Time and a reduced model of -Replicate. For this analysis other exposure samples were not included. For the antigen exposure analysis (day 0 compared to day 28 CAE, see FIG. 2A), some genes were filtered out which register as significantly different but which may be artifacts of the SMART-seq library construction; these fall along the arcs of a parabola in a volcano plot of the data. Lines were interpolated on the plot using genes along the arcs: between IL22 and WDR63 (negative) and between ALK and INBHE (positive). Genes with a perpendicular distance <1 to the lines were removed. DESeq2 adjusted counts were used to generate gene expression plots of NK associated molecules (see FIG. 2D). Statistics assessed by Mann-Whitney test (****P<0.0001, ***P<0.001, **P<0.01, *P<0.05).

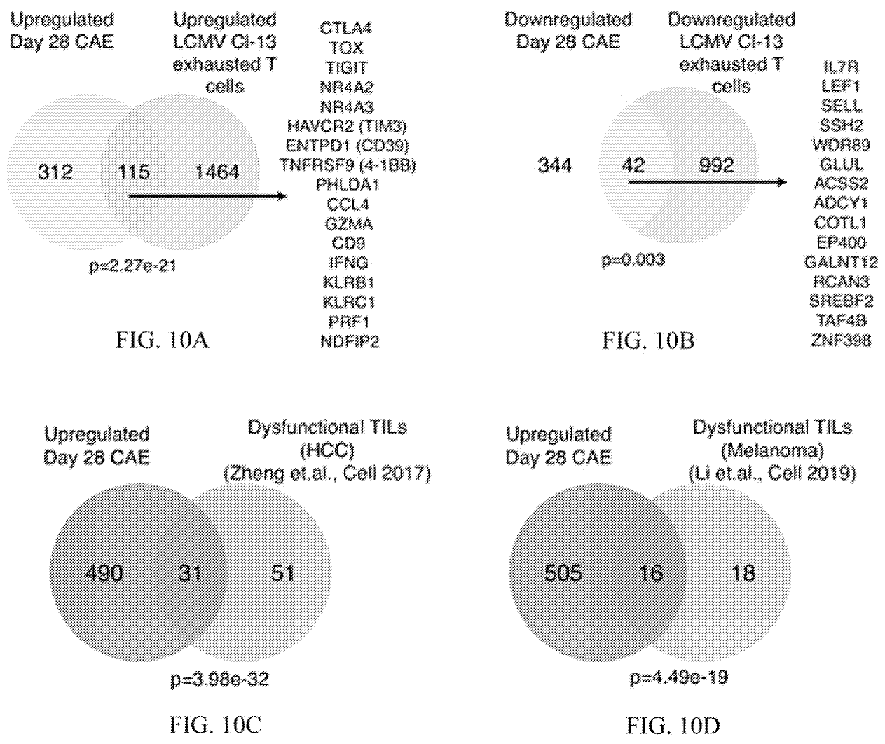

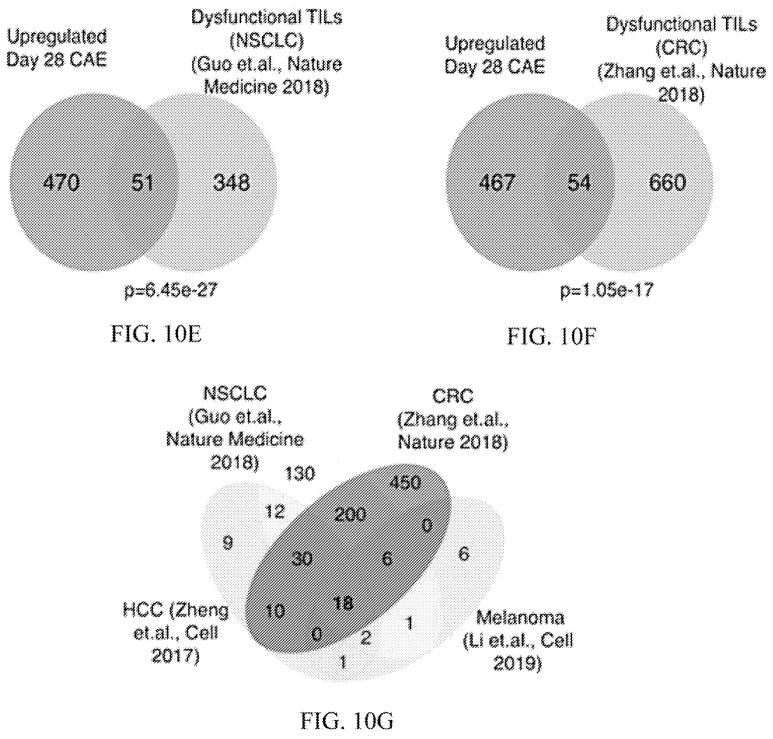

To contrast CAR+ and CAR− samples, transcripts per kilobase million (TPMs) were calculated for each gene using the bioinfokit.analys module in python. Gene lengths were calculated from the gene models used to run HTSeq, taking the maximum of all summed exon lengths across multiple isoforms as the length of the gene. For illustration purposes, outlying genes with high expression (>15,000) in surCARpos versus surCARneg plots were removed to more easily see where >99% of the genes fall on the correlation plot. However, all genes were included to make calculations, including spearman r (see FIG. 10H-10I).

Tracks were created for RNA-seq by pooling CAR+ samples across all replicates for control day 0 or CAE samples. BED files were filtered to remove alignments extending over 100 bp, primarily removing intron-spanning alignments. Coverage maps were created using BEDtools genomeCoverageBed -bg and these were adjusted by multiplying by the RPM coefficient. Resulting bedGraphs were converted to bigWigs using UCSC Genome Browser Tools' bedGraphToBigWig.

Bulk RNA-seq was compared to single-cell RNA-seq by taking all genes with significant differences in the single-cell data (between day 0 and day 20 CAE, identified using cellfishing.jl software) and rank-ordering them into ten deciles by log₂(day 20 CAE/day 0 control), then representing the bulk RNA-seq log₂(day 28 CAE/day 0 control) for each decile by box and whisker. Boxes are heated by the median value.

ATAC-seq: Omni ATAC-seq libraries were made as previously described (Corces et al., (2017) Nat Methods 14, 959-962). Briefly, nuclei were isolated from 30,000 sorted CD8+ surface CAR+ T cells, followed by the transposition reaction using Tn5 transposase (Illumina) for 30 minutes at 37° C. with 1000 rpm mixing. Purification of transposed DNA was completed with DNA Clean and Concentrator (Zymo) and fragments were barcoded with ATAC-seq indices (Buenrostro et al., (2013) Nat Methods 10, 1213-1218). Final libraries were double size selected using AMPure beads prior to sequencing. Paired-end sequencing (2×75 bp reads) was carried out on an Illumina NextSeq 500 platform.

Paired-end data were aligned to human genome assembly GRCh37/hg19 using bowtie2 v2.3.4.1 with parameters --local -X 1000. Resulting SAM files were converted to BAM and filtered for match quality using samtools view -q 5 -bS (samtools v1.1). BAM files across four NextSeq lanes were merged and sorted by read name using samtools merge -n, then PCR de-duplicated with PICARD MarkDuplicates REMOVE_DUPLICATES=True ASSUME_SORT_ORDER=queryname. BAM files were converted to BEDs using BEDtools bamToBed and processed to remove all alignments on chrM. Alignments with a mate distance under 100 bp were kept as sub-nucleosome fragment size signal and others were discarded.

For replicates R2 and R5, re-sequenced libraries were pooled using UNIX cat as follows:
R2 Day 0: 4-Day0-CD8-CARpos-R_S13, 4-Day0-CD8-CARpos-10-24-2018_S6
R2 CAE: 8-CAE-CD8-CARpos-R_S7, 8-CAE-CD8-CARpos-10-24-2018_S5)
R5 Day 0: 2-Day0-CD8-CARpos-REP5-ATAC-re_S17, 2-Day0-CD8-CARpos-REP5-ATAC_S17
R5 CAE: 4-CAE-CD8-CARpos-REP5-ATAC-re_S15, 4-CAE-CD8-CARpos-REP5-ATAC_S18

Peaks were called in the sub-nucleosome fragment fraction of alignments using MACS2 callpeak with parameters -s 42 -q 0.01 and no explicit background control sample. The FDR was subsequently controlled at 0.001.

Robust peak sets for control and CAE were identified in the following way. Peaks in either condition were combined across replicates, merging overlapping loci. Merged peaks without representation (BEDtools intersect) in all four replicates were removed.

To make track visualizations of the ATAC-seq data, an appropriate library size adjustment is necessary. DESeq2 was used to calculate size factors (coefficients for library size adjustment for each sample) from a set of pan-conditional peaks. The robust peak sets for control and CAE were combined, merging overlapping loci. Tag counts were calculated for all pan-conditional peaks across all samples and the resulting table was input to DESeq2 to estimate size factors and get adjusted tag counts at each peak. For each sample, sub-nucleosome sized fragment alignments were converted into a coverage map using BEDtools genomeCoverageBed -bg. Resulting bedGraph files were adjusted for library size by dividing coverage tallies by the DESeq2 size factors. Files were then sorted using UCSC Genome Browser Tools' bedSort and converted to bigwig format using bedGraphToBigWig.

To compare ATAC-seq to polyclonal RNA-seq, pan-conditional peaks were filtered to remove peaks overlapping ENCODE blacklisted regions. Remaining peaks were mapped to the nearest RefSeq transcript by TSS. The set of genes up- or down-regulated at FDR 0.05 in the antigen exposure contrast was used to identify mapped peaks, and their DESeq2-adjusted counts were plotted by box-and-whisker. Statistics assessed by Mann-Whitney.

Enriched motifs were identified in peaks specific to control day 0 or CAE using HOMER v4.6 findMotifsGenome.pl with command-line parameters -size 200-mask. Robust peak sets were filtered for any overlap with ENCODE blacklisted regions or with peaks from the other condition (e.g., control day 0 peaks without overlap to CAE peaks) using BEDtools intersect, and these specific peak sets were input to HOMER. The HOMER background (-bg) was set as robust peaks specific to the other condition.

To analyze the enrichment of Sox17 at ATAC-seq peaks, the Sox17 position weight matrix was downloaded from JASPAR (MA0867.2) and scanned against robust CAE-specific peaks (those without overlaps to ENCODE blacklisted regions or control day 0 peaks) or peaks common to control day 0 and CAE stimulation using PWMSCAN, with the FDR controlled at 1E-8. Peaks were divided into those with or without the motif and DESeq2-adjusted values are shown for these peak sets in box-and-whisker. Statistics assessed by Mann-Whitney.

Single-cell RNA-seq analysis: Sequencing data were aligned to the GRCh38 genome, filtered, and then barcodes and unique molecular identifiers were counted using the Cell Ranger v3.1.0 command cellranger count. Data were further analyzed in R using Seurat version 3.1.2 (Butler et al., (2018) Nat Biotechnol 36, 411-420; Stuart et al., (2019) Cell 177, 1888-1902.e1821). Briefly, genes that were not detected in at least 3 cells and cells with >5% mitochondrial reads were excluded, as well as cells that express <200 genes or >5000 genes. Data were normalized using sctransform (Hafemeister and Satija, (2019) Genome Biol 20, 296). PCA was performed on the most variable genes which were found based on average expression and variance. Clusters and UMAP were generated from the first 10 PCA dimensions using the default parameter settings in Seurat. Marker genes were determined using the FindAllMarkers function in Seurat where at least 25% of the cells must be expressing the gene. Sctransform normalized expression was used for the heatmap of marker genes, UMAP feature plots, and dot plots. Metascape was used with cluster marker genes for gene ontology analysis. Monocle 3 was used for trajectory analysis with the default parameter setting and 100 PCs. AddModuleScore was used to project expression of the dysfunction signature genes (N=30) onto the Monocle trajectory. Gene regulatory network inference was performed using the partial information decomposition algorithm, PIDC, on the top 500 variable genes (identified via Seurat) with a threshold for edge inclusion of 15%. Cellfishing.jl, a software that builds a database from single-cell data to then be queried, was used for differential expression analysis between single cell data sets (day 0 product versus day 20 CAE cells) with the default of 10 k-nearest neighbors (Sato et al., 2019). 1,834 genes were found to be differentially expressed. Data were analyzed using IPA (QIAGEN Inc., https://www.qiagenbioinformatics.com/products/ingenuitypathway-analysis). For IPA analysis, mitochondrial genes were filtered out and only genes with fold change >2 (N=1,442 genes) were included. Fold change was calculated as the number of cells at day 20 that upregulate the gene divided by the number of cells at day 20 that downregulate the gene compared to day 0 cells. NK-like T cells were identified using raw_counts [“KLRC1”,]>0 & raw_counts[“KLRB1”,]>0 & raw_counts[“CD3E”,]>0. Significant differences in changes in the NK-like T cell populations between WT and KO conditions were measured by Fisher's exact test.

To identify the 30 gene dysfunction signature, all genes differentially expressed between dysfunctional and nondysfunctional clusters were identified using Seurat's FindMarkers function. For donor ND388, differentially expressed genes were identified between dysfunctional clusters D20-1 and D20-4 versus non-dysfunctional clusters D20-2 and D20-3. This list was further filtered by log₂FC>0.64 and padj<1.0e22 (padj with Bonferroni correction using all genes in the dataset).

WT, SOX4 KO, and ID3 KO Seurat objects were combined for analysis using the merge function (for donor ND566) and WT and ID3 KO samples were combined for donor ND539. Genes that were not detected in at least 3 cells and cells with >5% mitochondrial reads were excluded, as well as cells that express <200 genes or >5000 genes. EPCAM expression (tumor marker) was used to identify a contaminating tumor cell cluster which was subsequently removed using seurat's subset function. CellCycleScoring was used to regress out cell cycle specific clustering using SCTransform vars.to.regress (S.Score, G2M.Score) function. SCT counts of the dysfunction signature genes (N=30) were averaged per cell to create the dysfunction score. Mann-Whitney U test was used to test significance of dysfunction score between WT and KO conditions.

To assess the expression of M5CAR in the scRNA-seq data, the cellranger reference was reindexed (mkref) by adding a single contig for the 627 bp WPRE sequence (a unique sequence in the CAR plasmid) to assembly GRCh38 of the human genome (the gene annotation GTF file was appended with CDS and exon entries spanning the entire sequence and gene id “Ligand”). To analyze expression of CAR and to determine the percent of cells expressing the CAR, data was pooled from three scRNA-seq experiments (ND388 day 20 CAE cells, ND538 and ND150 day 28 CAE cells). Cells belonging to the dysfunctional clusters and non-dysfunctional clusters were defined for each donor separately.

Single-cell TCR-seq analysis: Sequencing data was aligned to the vdj-GRCh38-alts-ensembl-3.1.0 genome and processed using the cellranger vdj command in Cell Ranger v3.1.0. To assess receptor persistence, a map of full-length receptor peptide sequences to cell barcodes was loaded at both time points from filtered coverage annotation (FCA) files. Cell barcodes associated with peptide sequences in common to both time points were screened against lists of cell barcodes that express CD8A at both time points; cells without persistent CD8A expression were removed. Remaining cells were screened against barcodes of cells that express KLRB1 at either day 0 or day 28, or not at all. Sankey plots of this distribution were created using the plotly library in R. Maps were also analyzed for the number of cell barcodes associated to each full length peptide sequence to insure that the data largely obey a one peptide: one barcode rule.

LCMV chronic viral infection data analysis: RNA-seq FASTQ files were downloaded from GEO submission GSE86881 for naïve CD8+ T cells (GSM2309810, GSM2309811) and exhausted CD8+ T cells (GSM2309812, GSM2309813, GSM2309814). FASTQ files were aligned to the mm10 reference genome using STAR and differentially expressed genes between naïve CD8+ T cells and exhausted CD8+ T cells were identified using DESeq2. Only genes with mouse to human homologs were overlapped with CAR T dysfunction gene signature. Homologs were obtained from the Mouse Genome Informatics (MGI) database.

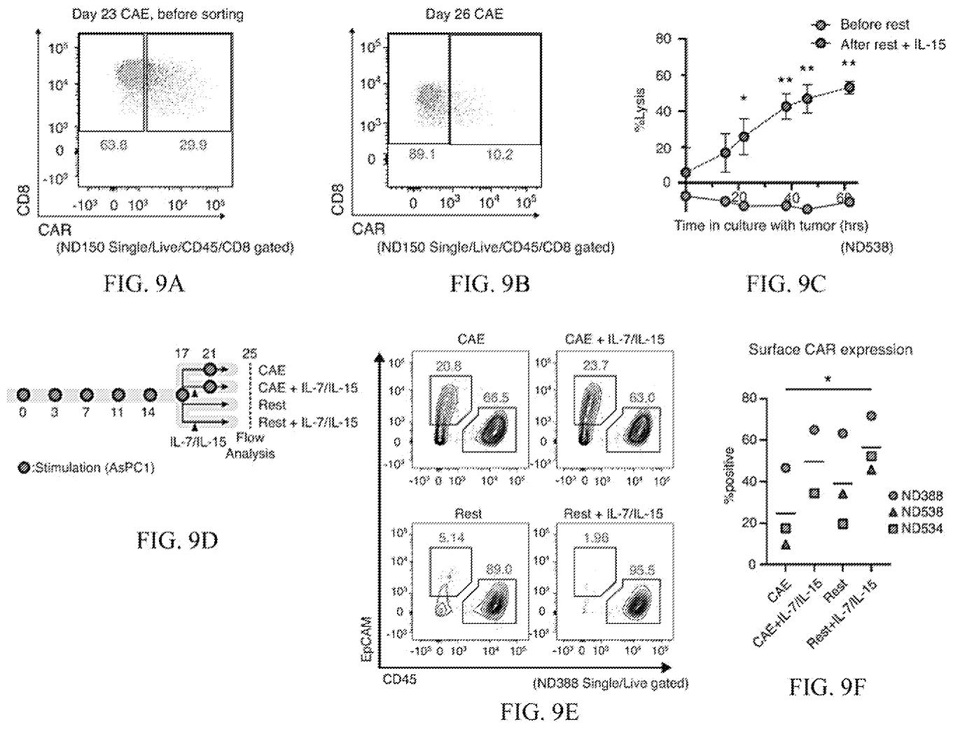

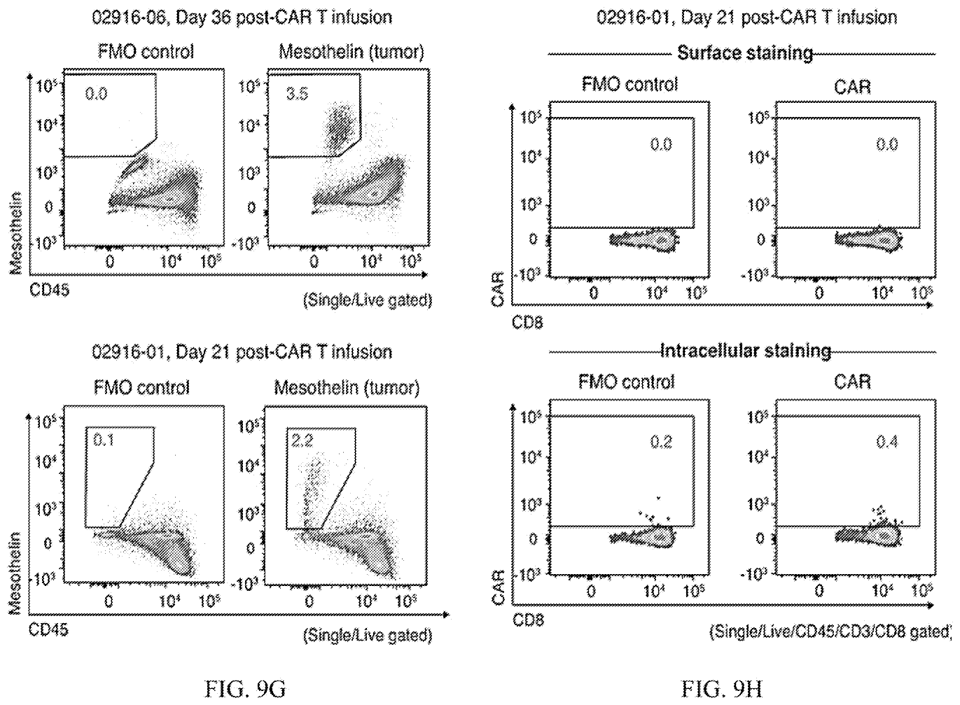

Human cancer TIL overlap analysis: The following published single-cell datasets were overlapped with the CAR T cell dysfunction gene signature. Colorectal cancer exhausted CD8 TIL associated genes were downloaded for the CD8_C07-LAYN specific genes (N=714 genes, including LAYN) (Zhang et al., (2018) Nature 564, 268-272). Non-small-cell lung cancer exhausted CD8 TIL associated genes were downloaded for the CD8-C6-LAYN specific genes (N=399 genes) (Guo et al., (2018) Nat Med 24, 978-985). Hepatocellular carcinoma exhausted CD8 TIL associated genes were downloaded (N=82 genes) from Zheng et al., (2017) Cell 169, 1342-1356 e1316). Melanoma exhausted CD8 TIL associated genes were obtained from FIG. 2B (genes most correlated with LAG3) and FIG. 9E (genes most correlated with HAVCR2) (N=34) (Li et al., (2019) Cell 176, 775-789 e718).

Guide RNAs targeting SOX and ID3: The following guide RNAs (gRNAs) were designed to target SOX4 (SEQ ID NOs: 1-5) and ID3 (SEQ ID NOs: 6-10) (FIG. 17): SOX4 gRNAs: GCTGGTGCAAGACCCCGAGT (SEQ ID NO: 1), CAAGATCCCTTTCATTCGAG (SEQ ID NO: 2), TGGTGTGGTCGCAGATCGAG (SEQ ID NO: 3), AGGAGGCGATTCCCAGCTCG (SEQ ID NO: 4), CGAGAACACGGAAGCGCTGC (SEQ ID NO: 5). ID3 gRNAs: TGGCTAAGCTGAGTGCCTCT (SEQ ID NO: 6), ATGTCGTCCAGCAAGCTCAG (SEQ ID NO: 7), TGTAGTCGATGACGCGCTGT (SEQ ID NO: 8), TGGCCAGACTGCGTTCCGAC (SEQ ID NO: 9), GGTGCGCGGCTGCTACGAGG (SEQ ID NO: 10).

Production of Human CRISPR-engineered CAR-T cells: Single guide RNA (sgRNA) sequences targeting ID3 and SOX4 were designed using CRISPick sgRNA designer (https:/portals.broadinstitute.org/gppx/crispick/public) and Benchling online software (https://www.benchling.com) and were synthesized by Integrated DNA Technologies (IDT). Two of five sgRNAs targeting each gene were selected for further experiments after pre-validation. Gene disruption, T cell activation, transduction, expansion, and knockout validation of ID3KO and SOX4KO M5 CAR T cells were performed following an optimized protocol previously described (Agarwal et al., (2021) J. Vis. Exp. https://doi.org/10.3791/62299). Briefly, CD4+ and CD8+T at 1: 1 ratio were incubated in OpTmizer T Cell expansion media (Gibco) supplemented with 5 ng/mL of huIL-7 and huIL-15 each (Preprotech) (OPT 7/15 media). After 24 h, cells were collected and resuspended at 1×10e⁸ cells/mL in P3 nucleofection solution (Lonza). The ribonucleoprotein (RNP) complexes were generated by incubating each sgRNA (5 mg per 10×10e⁶ cells) individually with the Cas9 nuclease (Aldevron, 10 mg per 10×10e⁶ cells) for 10 min at room temperature. Cells were electroporated in batches of 10×10e⁶ cells (100 mL) with a mixture of RNP complex plus 16.8 pmol of electroporation enhancer (IDT) into electroporation cuvettes (electroporation code EH111) in a 4D-Nucleofactor X-Unit (Lonza). After electroporation cells were grown in OPT 7/15 media at 5×10e⁶ cells/mL at 37° C. and activated 4 to 6 h later with anti-CD3/anti-CD28 monoclonal antibody-coated magnetic beads. After 24 h, T-cell were lentivirally transduced and expanded as described above.

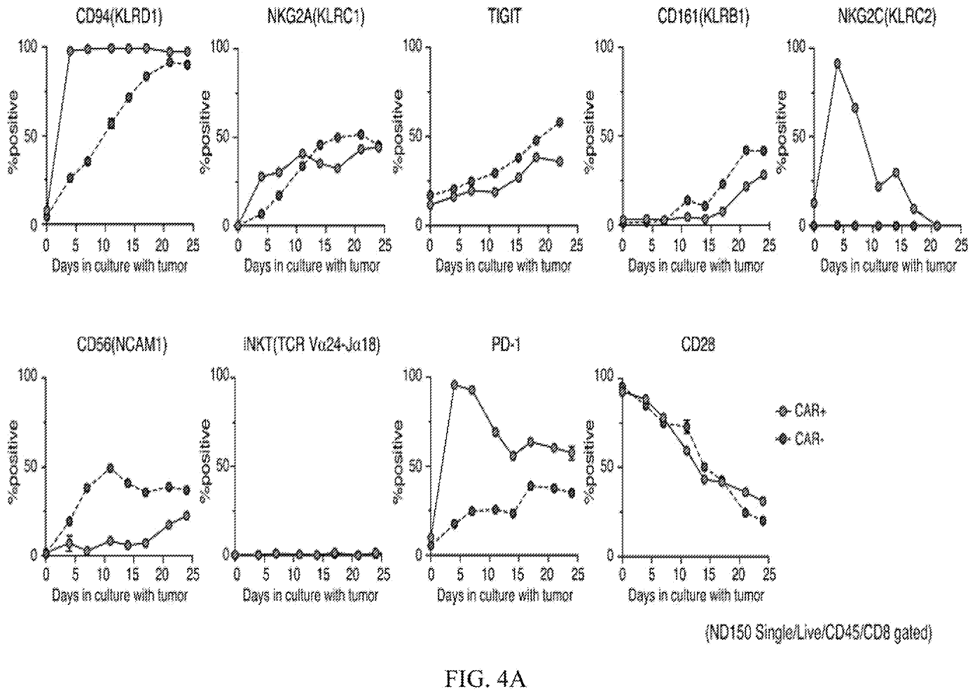

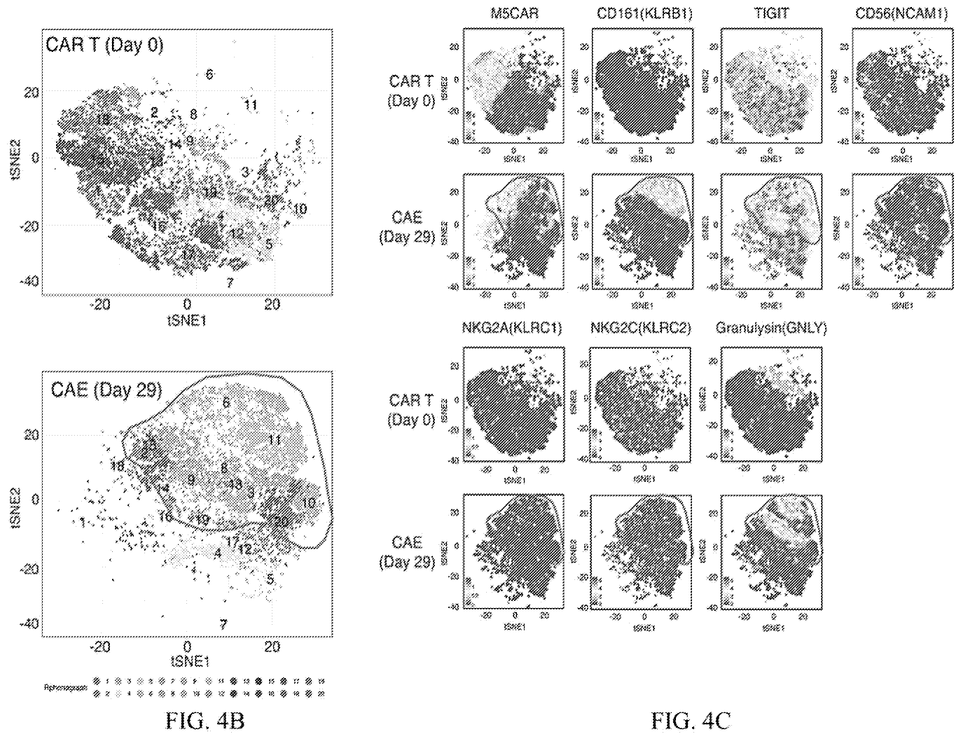

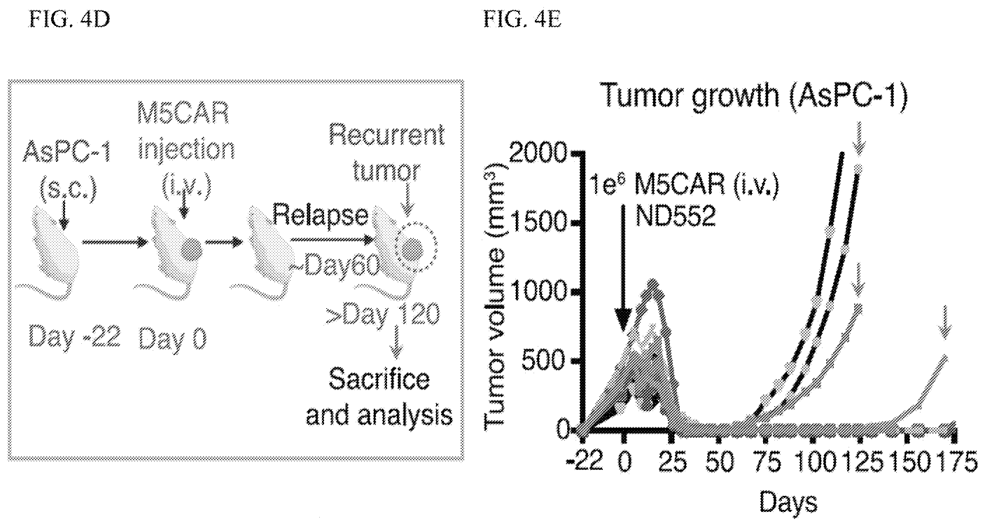

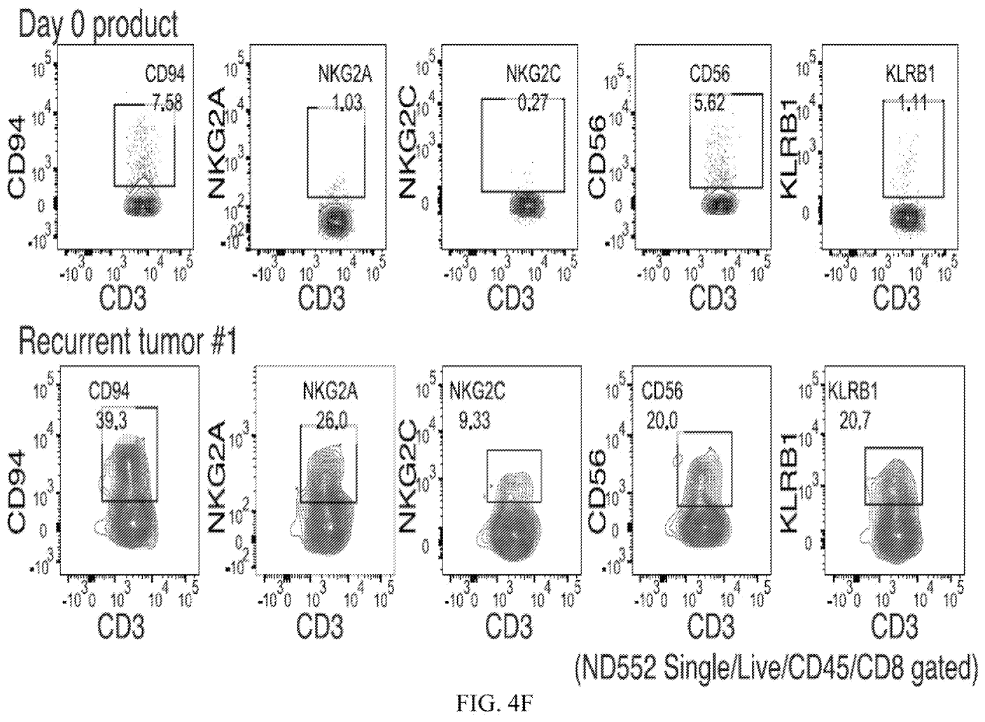

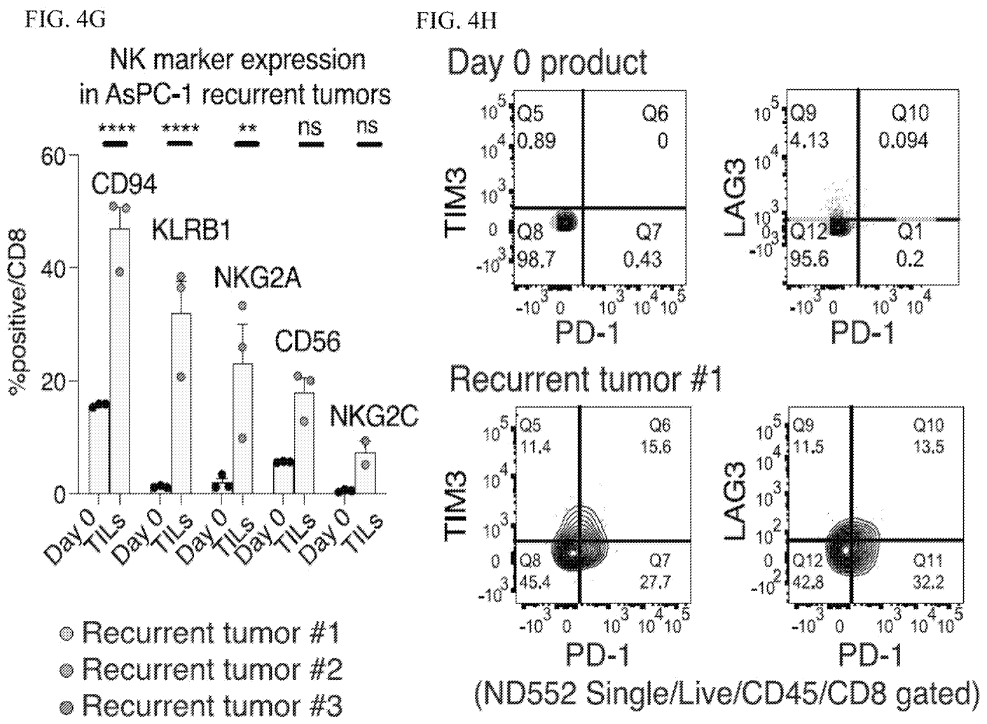

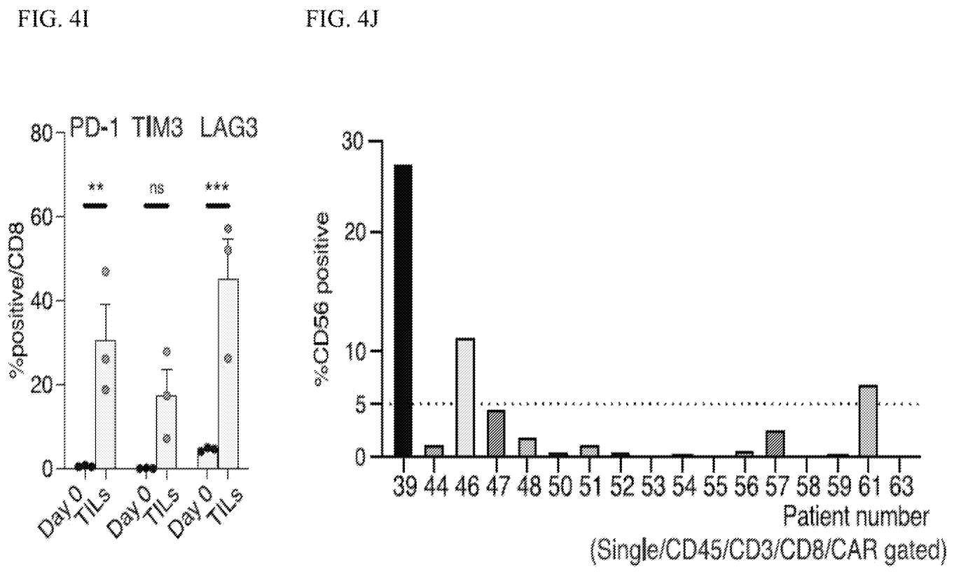

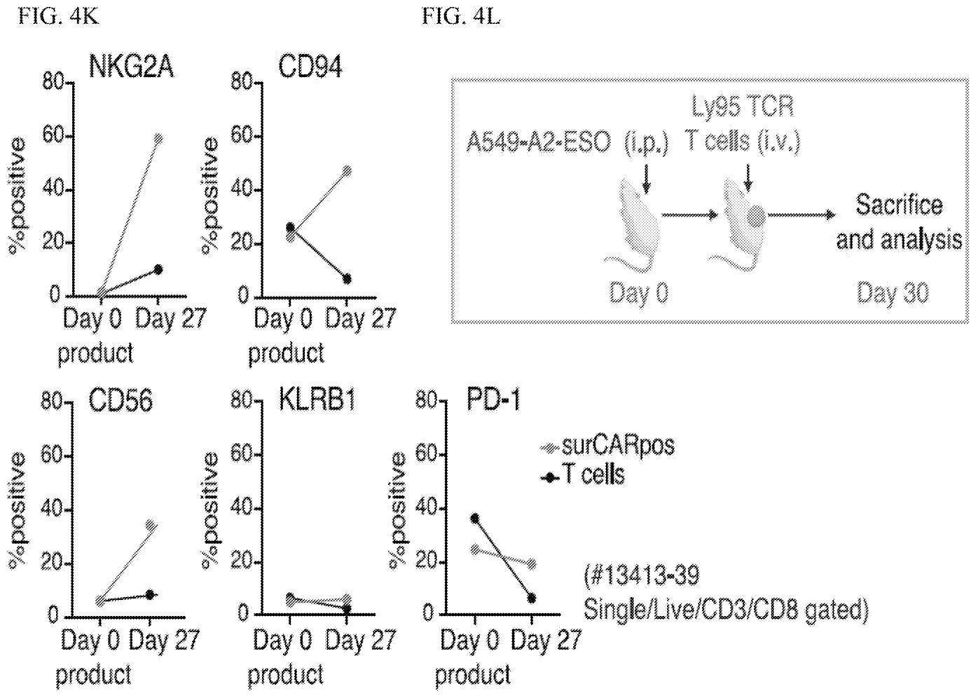

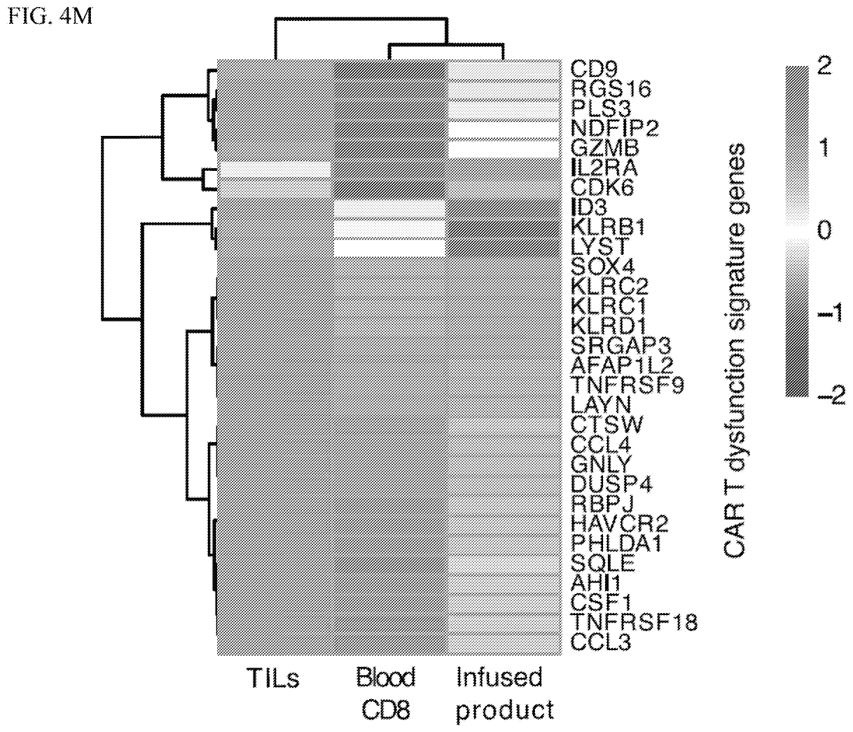

Since each target locus was defined by two sgRNA cut sites (spanned 100 and 130 bp for SOX4 and ID3, respectively), PCR primers and sequencing primers were designed to detect each target locus. LongAmp™ Taq 2× Master Mix (NEB) was used for target sequence amplification and used following manufacturer's protocol and NucleoSpin Gel and PCR Clean-up (Macherey-Nagel) was used for DNA purification. Analysis of gene editing efficiency was assessed by Sanger sequencing. Two sets of KO T cells were obtained per group: one bearing small insertions and deletions due to a single sgRNA hit, and a second population of CAR T cells bearing a large fragment deletion as a result of a double sgRNA hit. Synthego's Performance Analysis ICE (short for Inference of CRISPR Edits) tool, was used to calculate the editing efficiency (https://ice.synthego.com/[2021]). The schematic representation of the in vivo experiments of FIG. 4 and the knockout strategy in figure panel 20A were created using BioRender.com.

The results of the experiments are now described:

### Example 1: Establishment and Validation of an In Vitro Model of CAR T Dysfunction Induced by Prolonged and Continuous Antigen Exposure

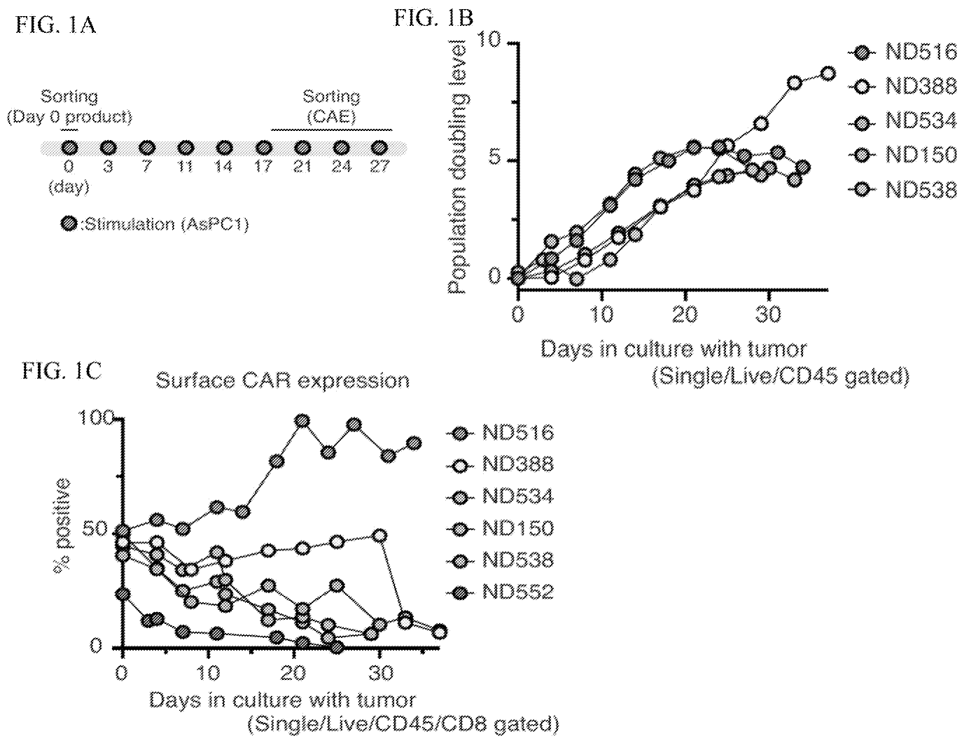

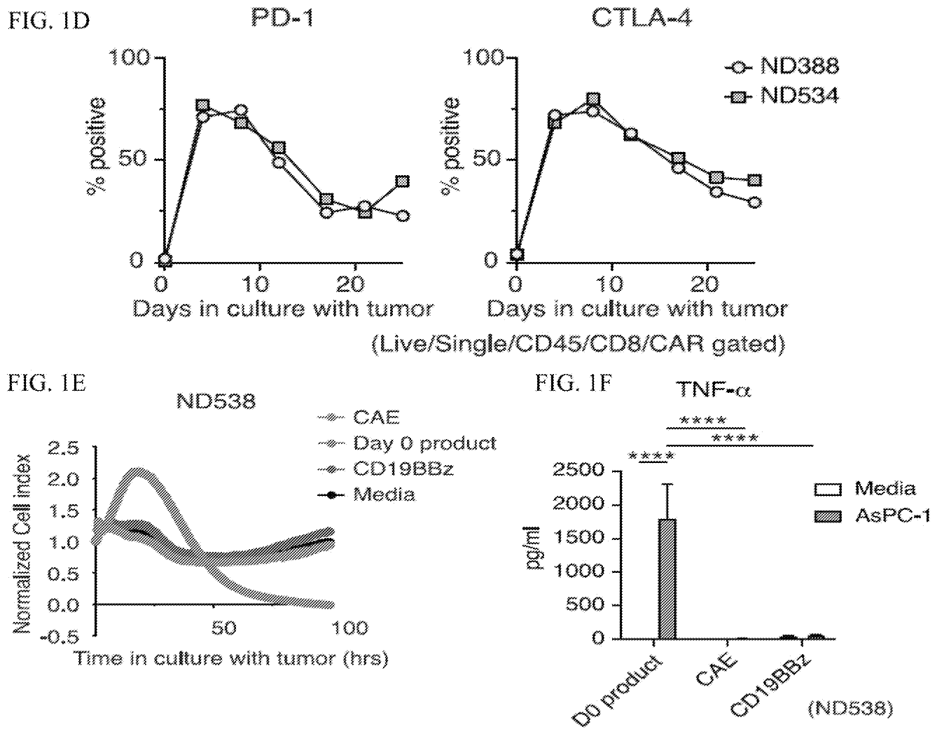

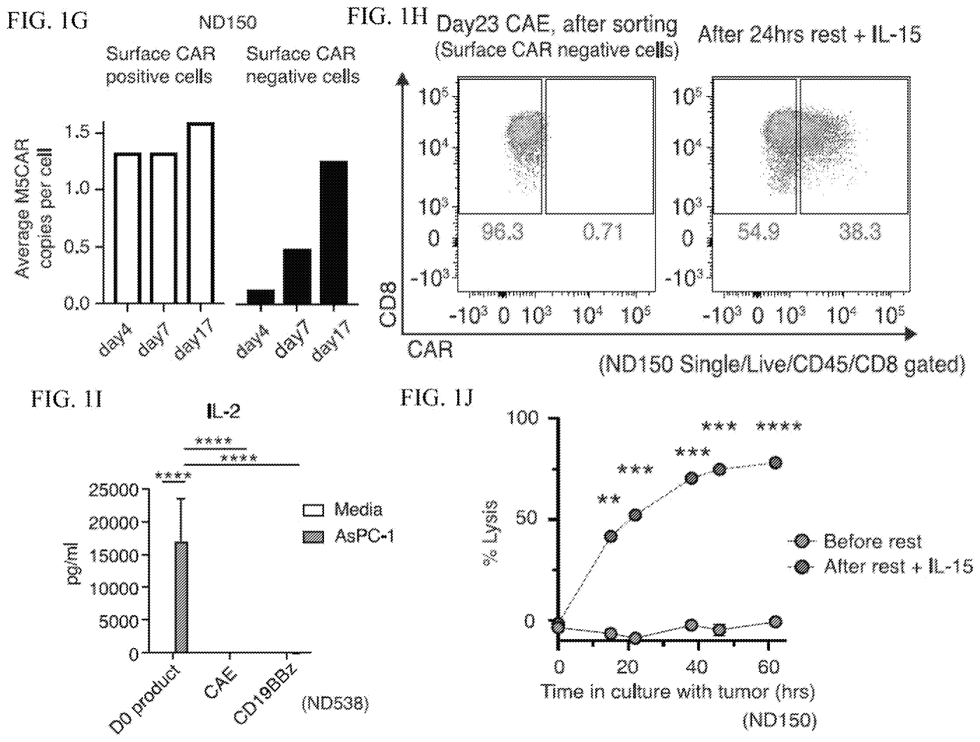

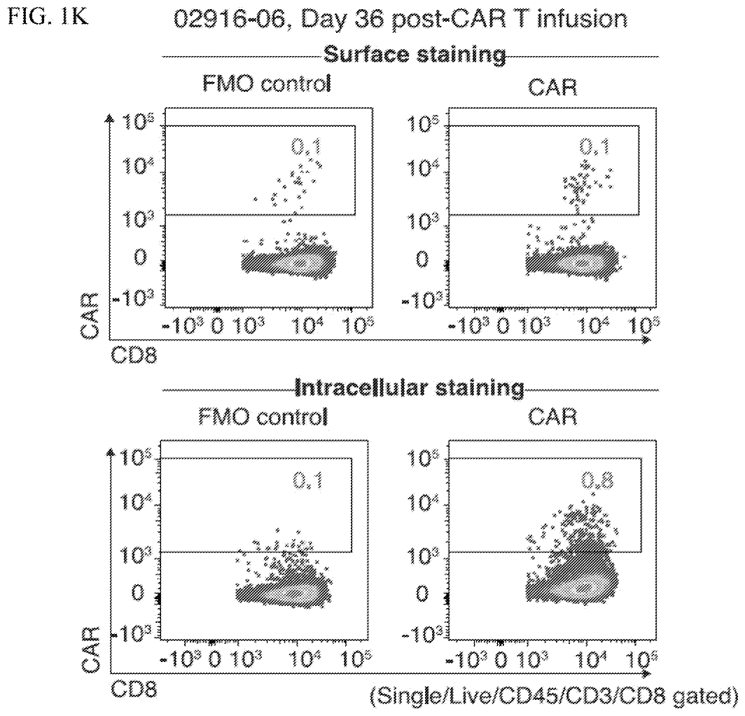

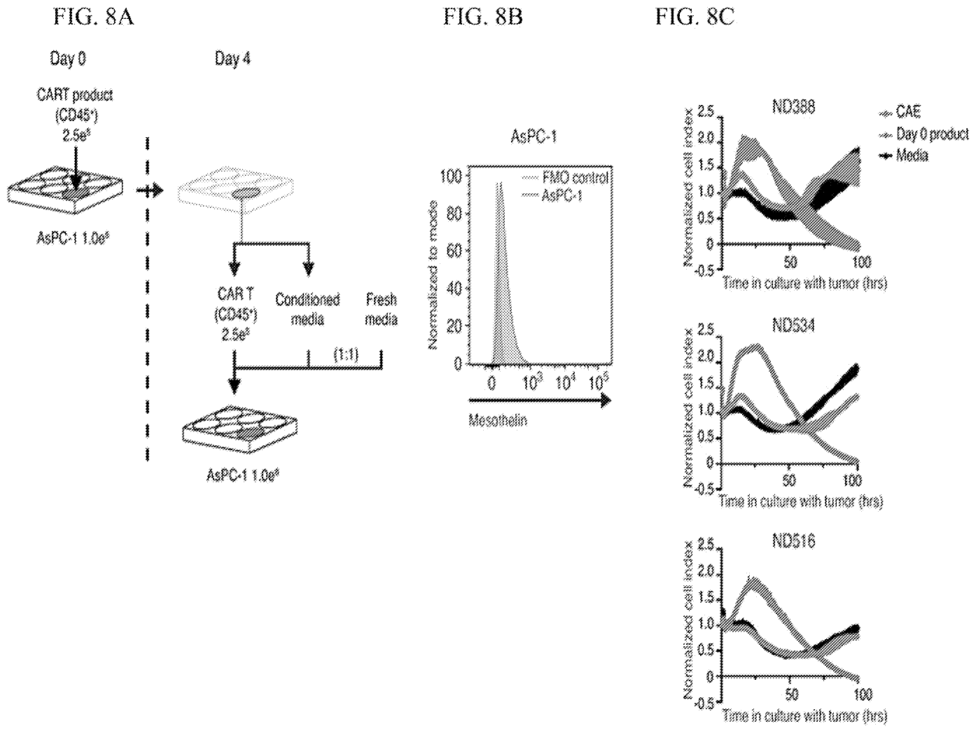

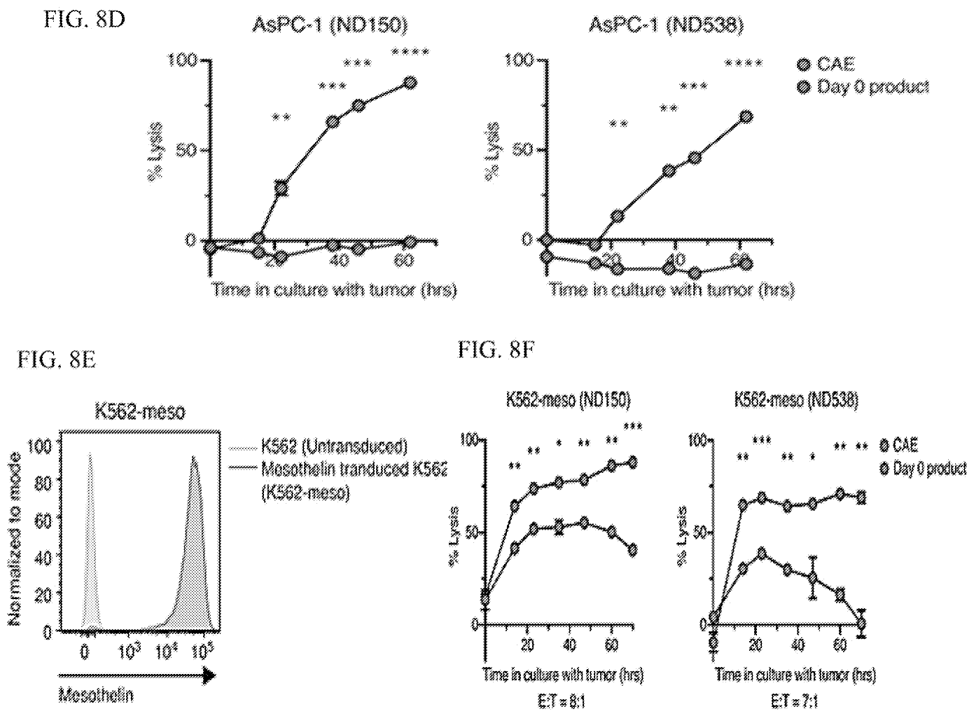

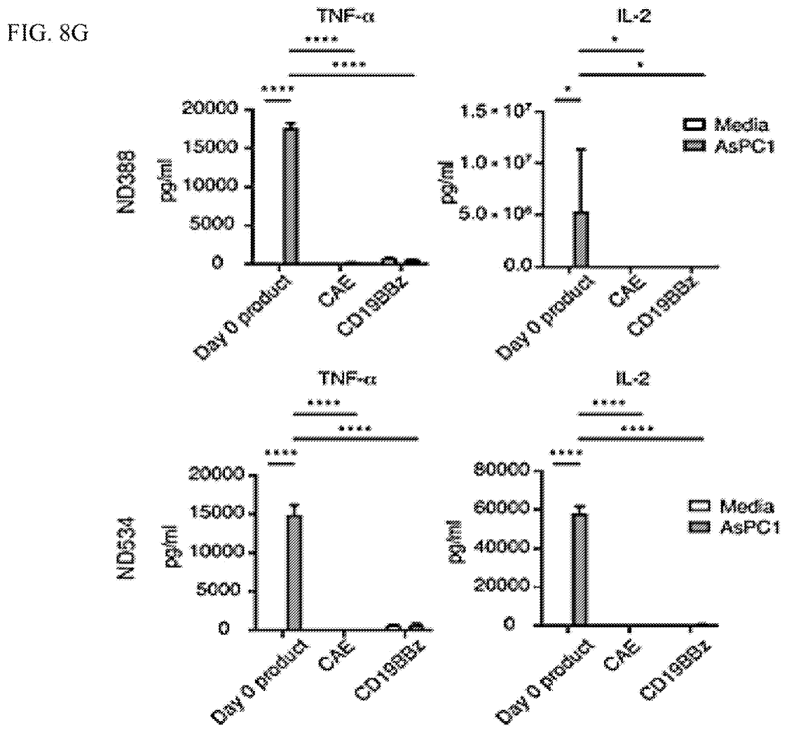

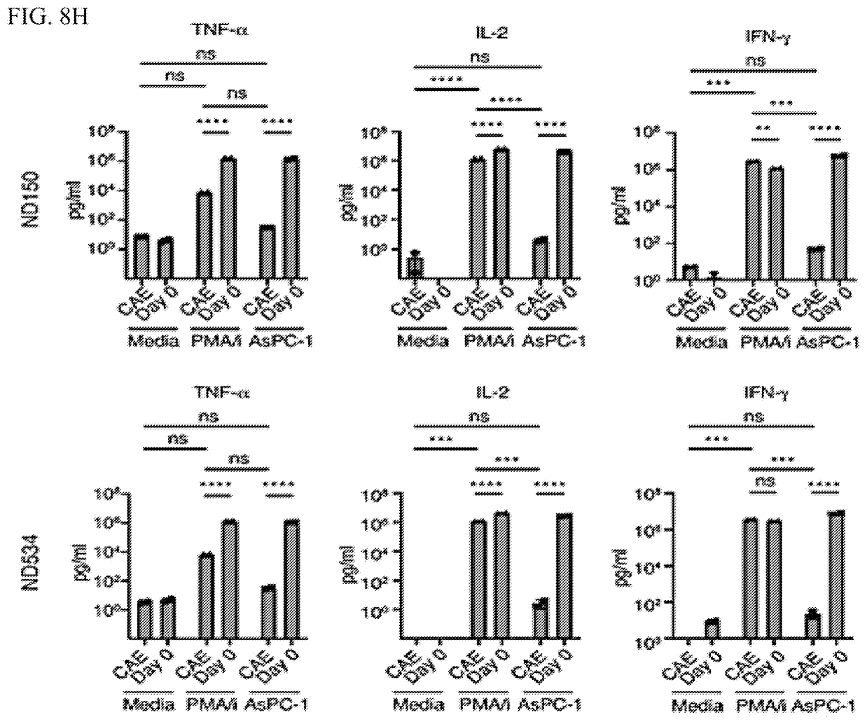

To gain a deeper understanding of CAR T cell exhaustion, an in vitro model was developed in which anti-mesothelin CAR (M5CAR) T cells were driven to a dysfunctional state through continuous antigen exposure (CAE). M5CAR comprises a human MSLN-binding scFv and CD8a hinge and transmembrane domains fused to 4-1BB and CD3-zeta cytoplasmic signaling domains. To achieve CAE, M5CAR T cells were manufactured from normal donor (ND) peripheral blood mononuclear cells (PBMCs) and repeatedly stimulated with a mesothelin-expressing pancreatic cancer cell line (AsPC-1) such that tumor cells were never cleared by the CAR T cells (FIG. 1A and FIGS. 8A-8B). AsPC-1 express low levels of mesothelin. It was reasoned that if CAR T cells were dysfunctional after prolonged CAE, they should exhibit reduced proliferation and cytokine production, extended expression of multiple immune checkpoints, and reduced ability to kill tumor cells. To test this, the population doubling of T cells transduced with M5CAR when co-cultured with continuous exposure to tumor was first measured by determining changes in CD45+ T cells, which discriminates T cells from residual tumor cells. After prolonged stimulation (20-35 days), M5CAR T cells lost or decreased doubling capacity, although this varied between donors with ND388 taking slightly longer to display a proliferative defect (FIG. 1B). Furthermore, although the viability of CAR T cells remained stable at 70% to 80%, the phenotype of apoptotic CAR T cells shifted from early apoptotic to late apoptotic after 18 days of CAE. Interestingly, when changes in the number of M5CAR T cells were directly measured, a reduction in CAR expression with prolonged CAE in all donors but ND516 was observed (FIG. 1C).

The expression of immune checkpoint inhibitors PD-1 and CTLA-4 was also measured on M5CAR T cells after CAE (FIG. 1D). Even though CD8+M5CAR T cells were at reduced numbers after CAE, sufficient cells from co-cultures were able to be sorted to perform comparison assays. At baseline CD8+M5CAR T cells did not express PD-1 or CTLA-4; however, this population exhibited high levels of PD-1 and CTLA-4 after initial stimulation (day 3) due to CAR T cell activation, and remained elevated above baseline as expected in dysfunctional T cells (FIG. 1D). In addition, CAR T cells upregulated the exhaustion marker TIM3 upon prolonged antigen stimulation. Furthermore, tumor cytotoxicity of CAR T cells following CAE was examined using the xCELLigence® Real-Time Cell Analyzer (FIGS. 1E and 8C) and Celligo Imaging Cytometer (FIG. 8D). While day 0 (unstimulated) CD8+M5CAR T cells eliminated tumor cells, day 28 CD8+M5CAR T cells and non-specific control CD8+CD19CAR (BBz)-positive T cells did not control tumor growth, revealing that surCARpos T cells become dysfunctional after tumor recognition and CAE. Additionally, loss of effector function was not specific to co-culture with the AsPC-1 tumor cell line; similar results were observed when CD8+M5CAR T cells were continuously stimulated with K562-meso, a human myelogenous leukemia cell line engineered to express mesothelin (FIGS. 8E and 8F). The effect of CAE on cytokine release from CD8+M5CAR T cells was measured following 28 days of exposure to AsPC-1 cells. While day 0 CD8+M5CAR T cells produced high levels of TNF-α and IL-2, in contrast, CAE CD8+M5CAR T cells and day 0 CD19BBz lacked cytokine production (FIGS. 1F, 1I and 8G). Together these data revealed that this in vitro model induces progressive CAR T cell dysfunction that is dependent on antigen recognition.

Next, it was examined whether this dysfunctional phenotype of CAR T cells in the model is specific to CAR signaling. CD8+M5CAR T cells were collected following 24 days of CAE, then stimulated with PMA+ ionomycin or AsPC-1 cells to measure cytokine production capacity. Both CAE and day 0 cells produced large amounts of TL-2 and IFN-γ after being stimulated with PMA+ ionomycin. However, when stimulated with AsPC-1 cells, cytokine production by the CAE cells was significantly reduced (FIG. 8H). CAE M5CAR T cells failed to secrete cytokines after prolonged CAR engagement, but still retained the ability to produce cytokines through pharmacologic stimulation by a CAR bypass mechanism, suggesting that downstream signaling remains intact.

### Example 2: Surface CAR Expression Dependent and Independent Mechanisms of Dysfunction

To further explore the decline in CAR expression with CAE, M5CAR T cells were sorted at 4, 7, and 17 days of CAE and genomic DNA (gDNA) of CAR positive and negative cell populations assayed. Cells sorted for CAR on the surface (surCARpos) have similar copies of M5 CAR gDNA throughout the CAE time course (FIG. 1G, left). Surprisingly, the population that was negative for surface CAR expression (surCARneg) exhibited increasing levels of M5CAR gDNA (FIG. 1G, right), suggesting that over time CAR T cells are expanding relative to untransduced T cells. By day 17, surCARpos and surCARneg populations expressed comparable levels of CAR gDNA, indicating that most surface CAR-negative cells are transduced CAR T cells with the CAR ligand internalized (FIG. 1G, left).

Hypofunctional mesothelin-directed CAR TILs isolated from mesothelin-expressing flank tumors in immunodeficient mice regain killing ability after rest and IL-2 treatment. Thus, it was tested whether CAE-induced loss of surface CAR is reversible in the present in vitro dysfunction model. Transduced M5 CAR T cells were cultured under CAE for 23 days (FIG. 9A), sorted for surCARneg cells (FIG. 1H, left) and then rested with fresh media plus IL-15 for a day. Both IL-2 and IL-15 are used in the clinical manufacturing process of CD19-directed CAR T cells; however, addition of IL-7 and IL-15 preserves a T cell population closely related to T-memory stem cells. After 24 hours of rest and antigen-independent IL-15 cytokine stimulation, 38% of surCARneg CD8+ T cells regained surface CAR expression (FIG. 1H). This suggests that T cells that have undergone CAE lose expression of CAR on the surface, and that this phenomenon is reversible.

The impact of CAE-induced surface CAR loss on M5CAR T cell effector function was investigated by measuring cell killing capacity. Polyclonal CD8+ T cells (10.2% surCARpos, FIG. 9B) collected after 26 days of CAE could not control tumor growth; however, 24 hour rest with IL-15 dramatically rescued their cytotoxic ability (FIG. 1J and FIG. 9C, second donor). Taken together, these results demonstrate that although loss of surface CAR expression is observed after prolonged CAE, M5CAR T cells can recover effector function and surface CAR expression with rest and IL-15 supplement.

A different approach was taken to examine whether rest and cytokine treatment rescue surface CAR expression and cytotoxicity of CAE CAR T cells. M5CAR T cells were continuously stimulated with AsPC-1 cells until day 17 at which point one group was rested with or without cytokine stimulation and another group was continuously stimulated with or without cytokines (FIG. 9D). Rested M5CAR T cells, with or without cytokine stimulation, were able to eliminate most of the AsPC-1 tumor cells at day 25 (EpCAM+CD45-), while M5CAR T cells with CAE, regardless of cytokine stimulation, were not (FIG. 9E). However, M5CAR T cells that received rest, cytokine treatment, or both rest and cytokine treatment displayed more surface CAR expression compared with CAE M5CAR T cells (FIG. 9F). Taken together, these results suggest that, although loss of surface CAR expression drives M5CAR T cell dysfunction, M5CAR T cells can recover effector function and surface CAR expression with rest and IL-15 supplement. Additionally, cytokine treatment alone rescues MSCAR expression on the cell surface, but MSCAR T cell killing is not restored. Therefore, surface CAR expression is necessary, but not sufficient for restoring M5 CAR T effector function.

Having demonstrated loss of surface expression of the MSCAR in vitro, the in vivo relevance of this phenomenon in the human tumor microenvironment was examined next. Peritoneal/pleural fluid samples collected after MSCAR T cell infusion were collected from two ovarian cancer patients (02916-01; day 21 and 02916-06; day 36) with peritoneal/pleural dissemination enrolled on a MSCAR T cell trial (NCT03054298). Tumor cells (identified by expression of mesothelin, FIG. 9G) and MSCAR CD8+ T cells were identified at days 21 (patient 02916-01) and 36 (patient 02916-06) post-CAR infusion (FIGS. 1K and 9H). Although the levels of MSCAR T cells in the peritoneal/pleural fluid were low as determined by qPCR, CAR T cells were able to be detected by flow cytometry. Notably, the frequency of intracellular CAR positive T cells (FIG. 1K, bottom right), which represent both surCARpos and surCARneg T cells, was higher than surCARpos T cells (FIG. 1K, top right), confirming that MSCAR T cells exhibited reduced expression of CAR on the cell surface in the human tumor microenvironment (FIG. 1K, FIG. 9H). In summary, CAE results in a reversible state of dysfunction of MSCAR T cells through loss of surface CAR expression, and quite unexpectedly, dysfunction as manifested by impaired cytotoxicity and cytokine secretion is also observed in cells that retain surface CAR expression.

### Example 3: Transcriptional Dynamics of Dysfunctional CAR T Cells

To better understand the mechanisms driving the loss of CAR T effector function in cells that express M5CAR on the cell surface, RNA-seq was performed on CD8+ surCARpos day 0 product and day 28 CAE surCARpos cells. This identified 1,038 differentially expressed genes (521 upregulated and 517 downregulated) in CAE surCARpos cells (FIG. 2A). In parallel, RNA-seq was performed on day 0 and day 28 CAE surCARneg CD8+ T cells (comprising both untransduced T cells and internalized CAR T cells). There was strong correlation of the gene expression signatures for surCARpos and sur-CARneg populations (FIG. 2B), suggesting that CAR T cells acquire the dysregulation signature before developing impaired expression of surface CAR. Since the phenotypic studies were performed in surCARpos cells (see FIG. 1) and the mechanisms of dysfunction in this population are unexplored, the remainder of the bulk RNA-seq analyses focused on this population.

To ascertain how well the model correlates with established in vivo models of T cell exhaustion, differentially expressed genes were compared between naïve T cells and exhausted T cells in the mouse model of chronic lymphocytic choriomeningitis virus (LCMV) infection (Pauken et al., (2016) Science 354, 1160-1165) with the present in vitro model of CAR T dysregulation. 27% of genes upregulated in CAE CD8+ surCARpos T cells overlapped with genes upregulated in exhausted T cells (115 gene overlap; p=2.27e-21), including genes implicated in the T cell exhaustion phenotype [CTLA4, TOX, TIGIT, NR4A2, NR4A3, HAVCR2 (TIM3), ENTPD1 (CD39), TNFRSF9 (4-1BB)] (FIG. 10A). There was also significant overlap (p=0.003) between genes downregulated in CAE and exhausted T cells (42 gene overlap), which included genes known to be expressed in naïve or memory CD8+ T cells (IL7R, LEF1, SELL, FIG. 10B). Further, GSEA analysis of the data with the four transient states of T cell exhaustion identified in the LCMV mouse model revealed significant enrichment with the intermediate and terminally exhausted T cell populations, indicating that the model recapitulates features of the later stages of T cell exhaustion in mouse T cells.

The present model was also compared to tumor-infiltrating lymphocytes (TILs), a second model of T cell exhaustion/dysfunction. The single-cell RNA-seq gene signatures of dysfunctional human CD8+ TILs isolated from patients with melanoma (Li et al., (2019) Cell 176, 775-789.e718), hepatocellular carcinoma (Zheng et al., (2017) Cell 169, 1342-1356.e1316), colorectal (Zhang et al., (2018) Nature 564, 268-272), and non-small cell lung cancer (Guo et al., (2018) Nat Med 24, 978-985) significantly overlapped with genes upregulated in CAE surCARpos T cells (FIGS. 10C-10F). To obtain a stringent list of dysfunctional TIL marker genes, datasets from the four cancer types above were overlapped, and a common group of 18 TIL marker genes was found (FIG. 10G), including genes known to be associated with T cell exhaustion (LAG3, PDCD1, CTLA4, HAVCR2) (Wherry (2011) Nat Immunol 12, 492-499). To determine how applicable this signature is to other CARs, we performed GSEA analysis of the exhaustion signature curated in GD2-directed CARs (Lynn et al., 2019). Genes upregulated in the exhausted CD8+GD2 CAR T cells were significantly enriched with genes up in day 28 CAE M5CAR T cells, suggesting that at least some of the signaling observed in the 4-1BB mesothelin-directed dysfunctional CAR T cells is conserved in the exhausted GD2-28z CAR T cells. Taken together, these analyses provide further evidence that the in vitro model of CAR T cell dysfunction presented herein aligns with in vivo human and mouse models of T cell exhaustion and dysfunction.

To further illuminate the biological functions of the entire dysregulated gene expression signature identified in FIG. 2A, Ingenuity Pathway Analysis (IPA) was performed. T cell exhaustion, PD-1/PD-L1 cancer immunotherapy, and CTLA4 signaling pathways were enriched (FIG. 2C). Interestingly, several pathways related to natural killer cells (NK cells), including crosstalk between dendritic cells and NK cells, communication between innate and adaptive immune cells, and NK cell signaling were also enriched in the gene expression signature of CAE CD8+ surCARpos T cells (FIG. 2C). In fact, multiple NK receptors were upregulated in CAE CD8+ surCARpos T cells, including KLRC1, KLRC2, KLRC3, KLRB1, KLRD1, and KIR2DL4 (FIG. 2D). αβ T cells often upregulate receptors constitutively expressed by NK cells, potentially due to chronic activation by antigens and cytokines. For example, CD8+ T cells in the inflamed intestine of patients with celiac disease and peripheral blood CD8+ effector memory T cells in healthy individuals possibly induced by prior infections undergo a re-programming to express CD94, NKG2A, NKG2C, KIR2DL4, and other NK receptors, as observed in the dysfunctional CAR T cells. To identify whether CAE drives a similar gene expression program in CD4+ T cells, RNA-seq was performed on day 0 and day 28 CAE surCARpos CD4+ T cells. Significant overlap was found between the CD4+ and CD8+ T cell signatures following CAE, including the upregulation of NK receptors (KLRB1, KLRC1, KLRC2, KLRC3, KLRD1) and other genes in our signature including GNLY, LAYN, CD9, PHLDA1, SOX4, and TNFRSF9.

To better understand how gene expression changes over time in the model, RNA-seq was performed on CAE surCARpos T cells at day 16 (a middle time point, in replicate). Genes were identified that show changes in expression between day 0, 16, and 28, illustrating distinct patterns of transcription (FIG. 2E). For example, many NK receptors and exhaustion markers gradually turn on, with moderate expression by day 16 and highest expression by day 28 (cluster 5: KLRD1, KLRC1, KLRC2, KLRC3, TOX, HAVCR2, TIGIT), while other markers remain off or lowly expressed until dramatic upregulation at day 28 (cluster 4: KLRB1, KLRK1). Cluster 6 represents genes that display robust activation early with slight downregulation by day 28, which includes inhibitory molecules (CTLA4, LAG3), genes encoding chemokines (CCL3, CCL4, CXCL8), cytotoxic molecules (PRF1, GZMB, NKG7), and a group of T cell activation genes. In parallel, RNA-seq was performed on day 0 and CAE surCARneg CD8+ T cells (comprising both untransduced T cells and internalized CAR T cells). There was strong correlation of the gene expression signatures for surCARpos and surCARneg populations (FIGS. 10H and 10I), suggesting that CAR T cells acquire the dysregulation signature before developing impaired expression of surface CAR.

To identify potential transcription factors that control the dysregulated gene expression signature in CAE surCARpos T cells, IPA's upstream regulator analysis software was used, which allows for the identification of transcription factors that can induce the gene expression changes observed in the dataset. To narrow down the list of potential candidates, transcription factors were selected that were themselves dysregulated upon CAE (FIG. 2F). This list includes transcription factors that are upregulated (EGR1, ID3, SOX4, RBPJ), as well as downregulated (KLF2, BCL6, LEF1) in CAE surCARpos cells. Interestingly, upregulated ID3 and RBPJ are predicted to regulate the dysfunctional CD8 signature in melanoma TILs, while downregulated KLF2 is associated with the cytotoxic CD8+ T cell signature.

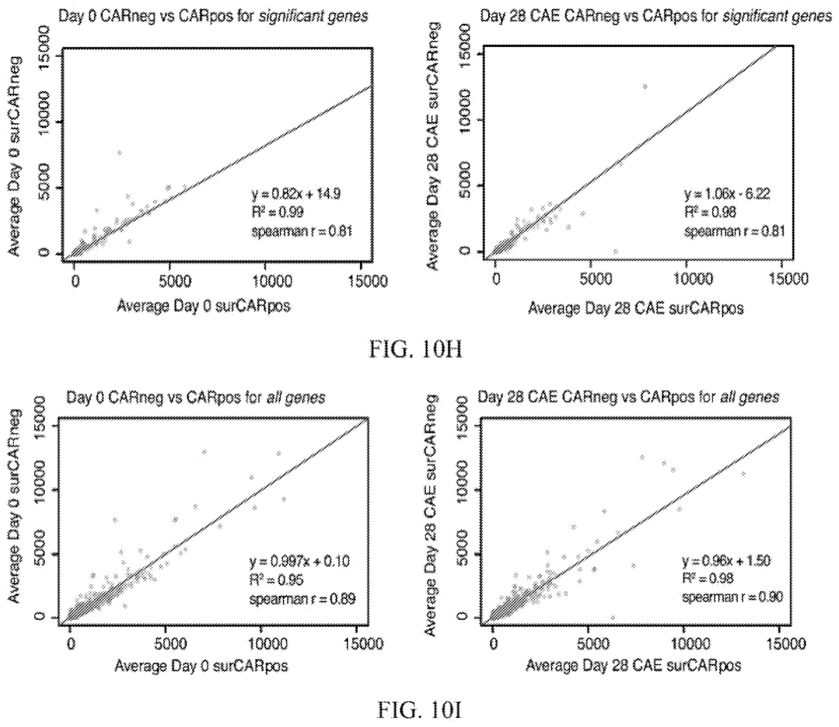

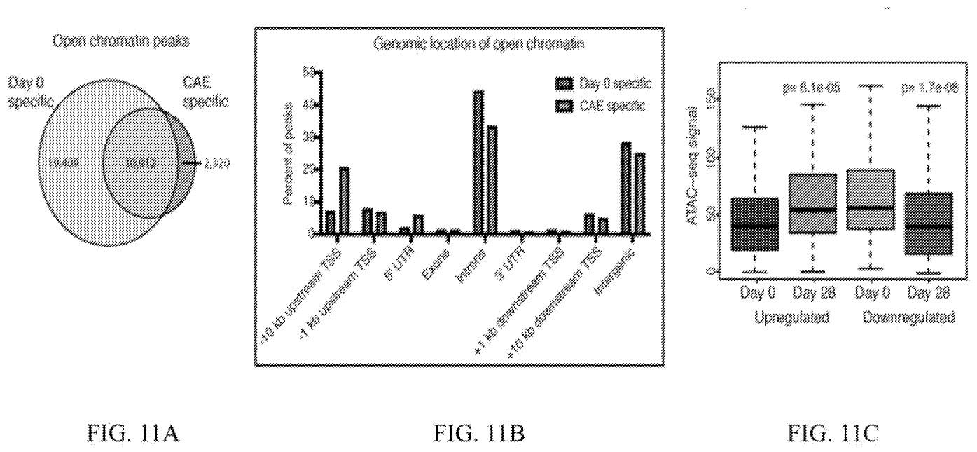

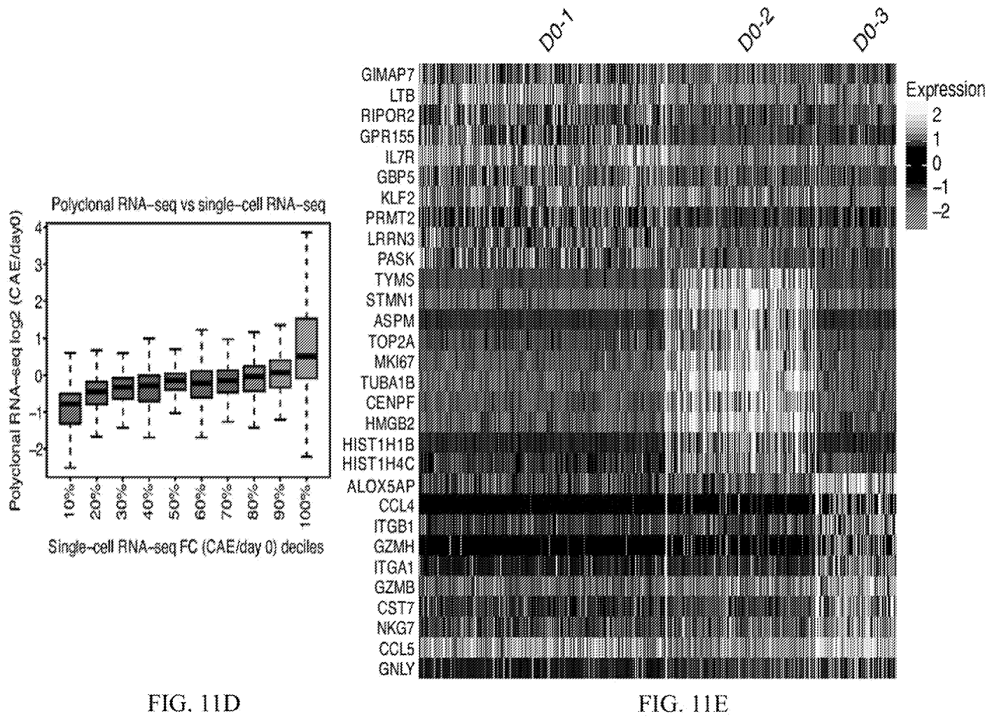

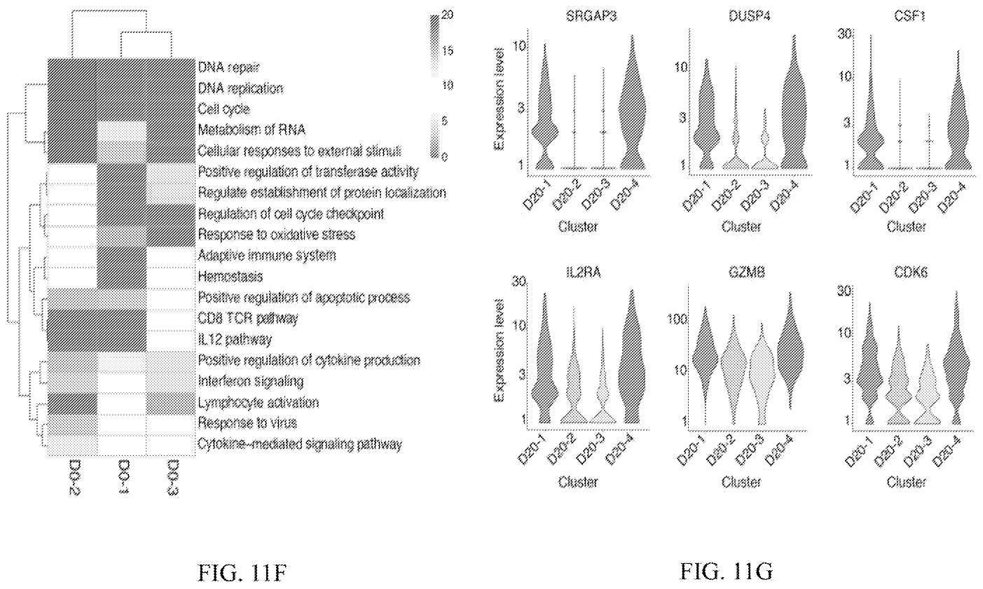

ATAC-seq (assay for transposase-accessible chromatin with sequencing) was performed to explore CAE specific regulatory changes. Overall, there was closing of chromatin upon CAE, with 30,321 open chromatin regions (OCRs) in day 0 and 13,232 OCRs in CAE surCARpos T cells (FIG. 11A). Although many open sites in day 0 close upon CAE, 2,320 regions are uniquely open in CAE surCARpos T cells, and these sites are mostly in introns (33.4%), intergenic (25%), and promoter regions (27.4%, <10 kb upstream from TSS) consistent with a regulatory role (FIG. 11B). In fact, compared to day 0, 5′ UTR and promoters are the most enriched locations for chromatin opening upon CAE, suggesting CAE cells having a unique epigenetic landscape. RNA-seq and ATAC-seq datasets were integrated in order to gain insights into how chromatin organization impacts gene expression in CAE surCARpos T cells. Genes upregulated in CAE have an opening of chromatin, while genes downregulated in CAE have a closing of chromatin (FIG. 11C). For example, the upregulated gene ID3 and the downregulated gene KLF2 displayed opening and closing of chromatin at their promoter regions, respectively (FIGS. 2G-2H). These data suggest that CAE results in a remodeling of chromatin that includes closing of chromatin at intronic regions and opening at gene promoters.

To determine if the epigenetic landscape of the dysfunctional CAR T cells is similar to TCR-mediated exhaustion, ATAC-seq datasets from exhausted human PD1-high TILs (Philip et al., (2017) Nature 545, 452-456. were queried. Chromatin sites opening in day 28 CAE cells were also open in exhausted TILs. Closing of chromatin was observed in day 28 CAE cells at CD5, CD28, and TCF7, similar to PD1-high human TILs or dysfunctional mouse T cells, as previously reported (Philip et al., (2017) Nature 545, 452-456).

### Example 4: Single-Cell Analysis of CAE CD8+ T Cells Reveals Co-Expression of Dysfunction Signature Genes

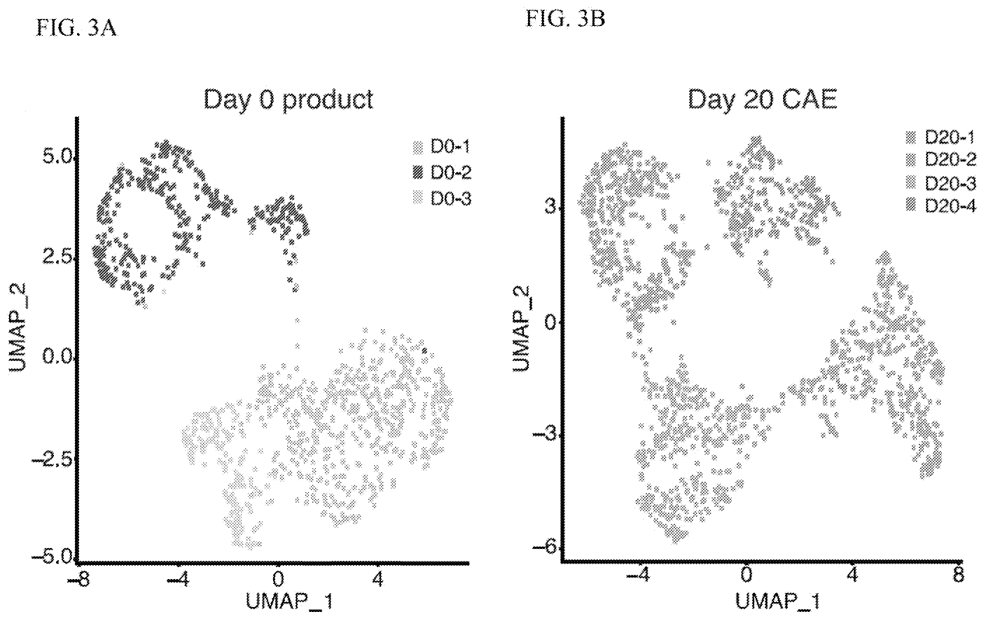

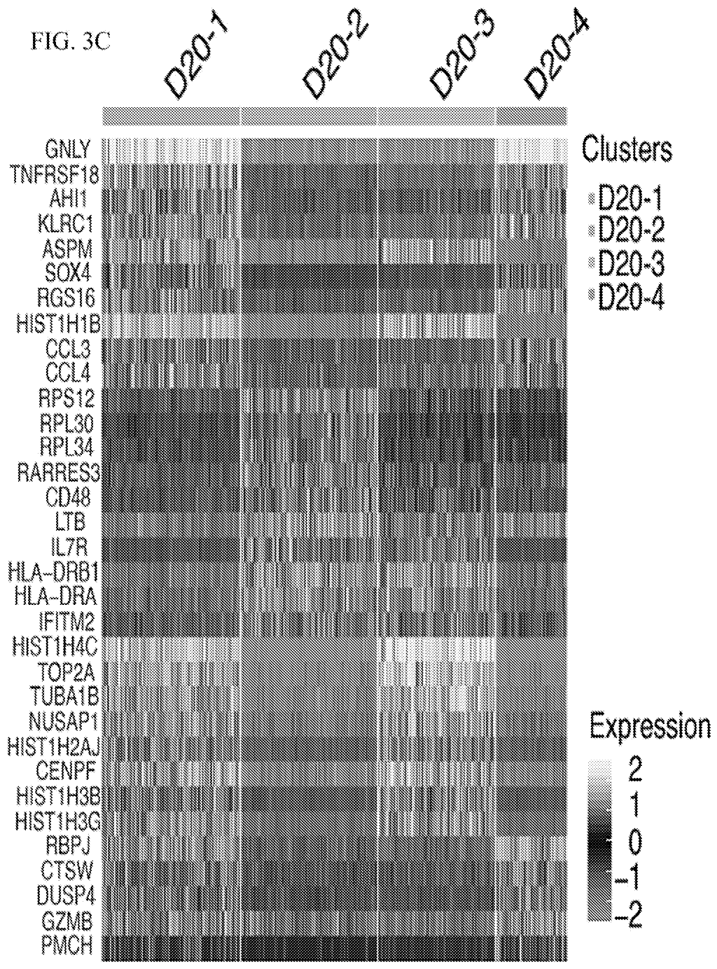

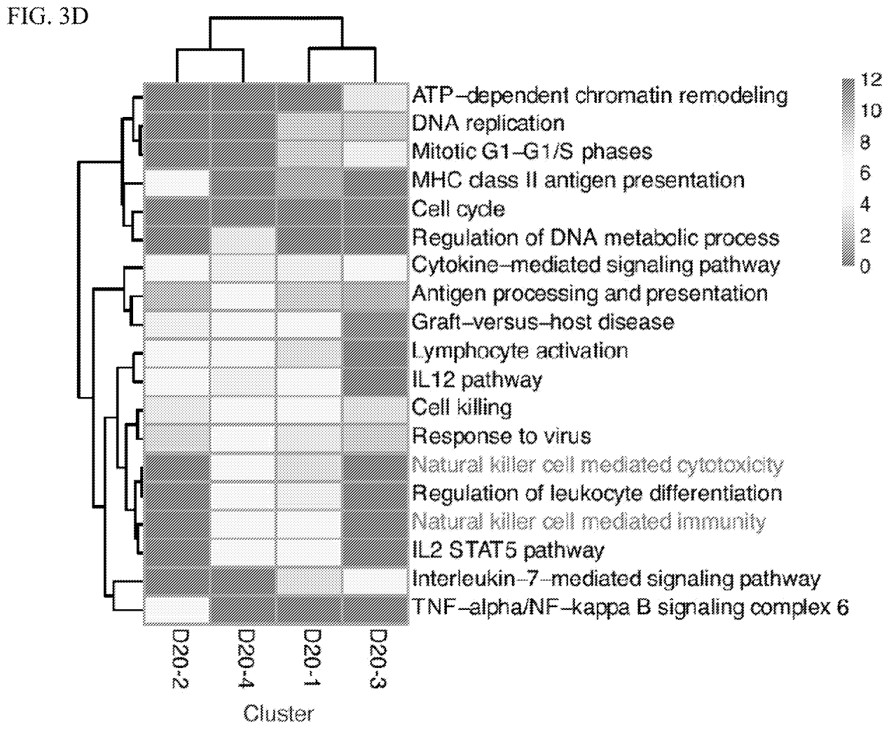

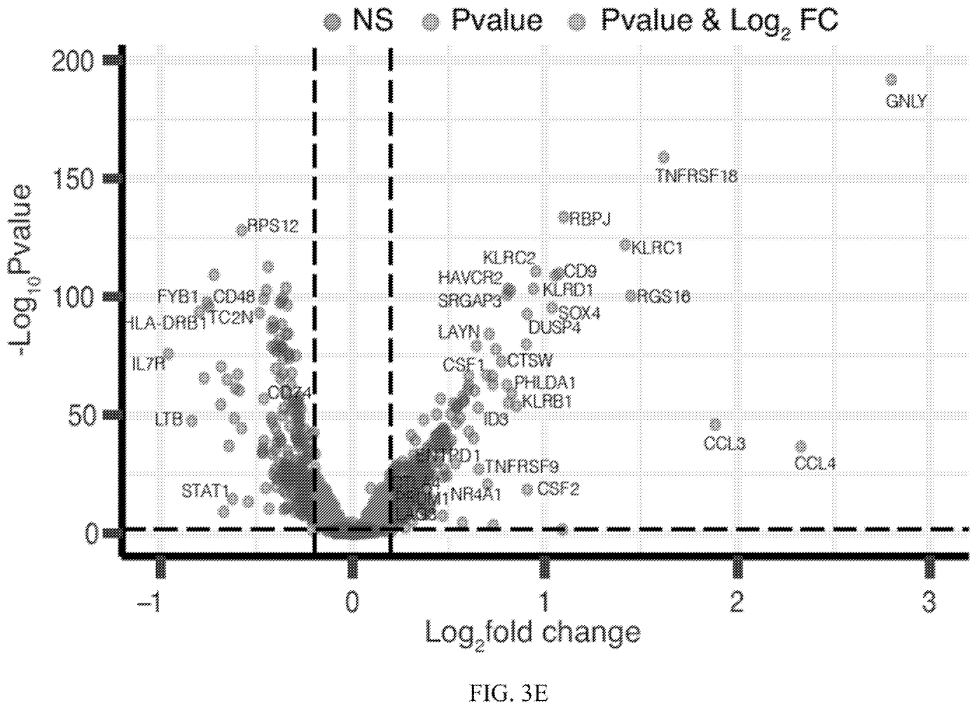

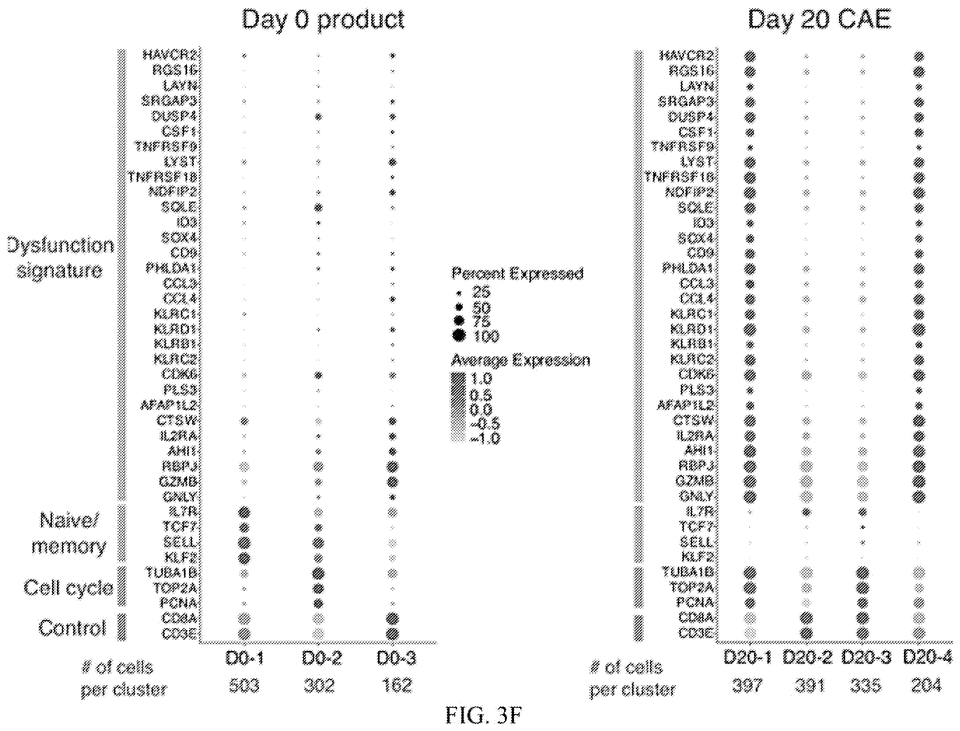

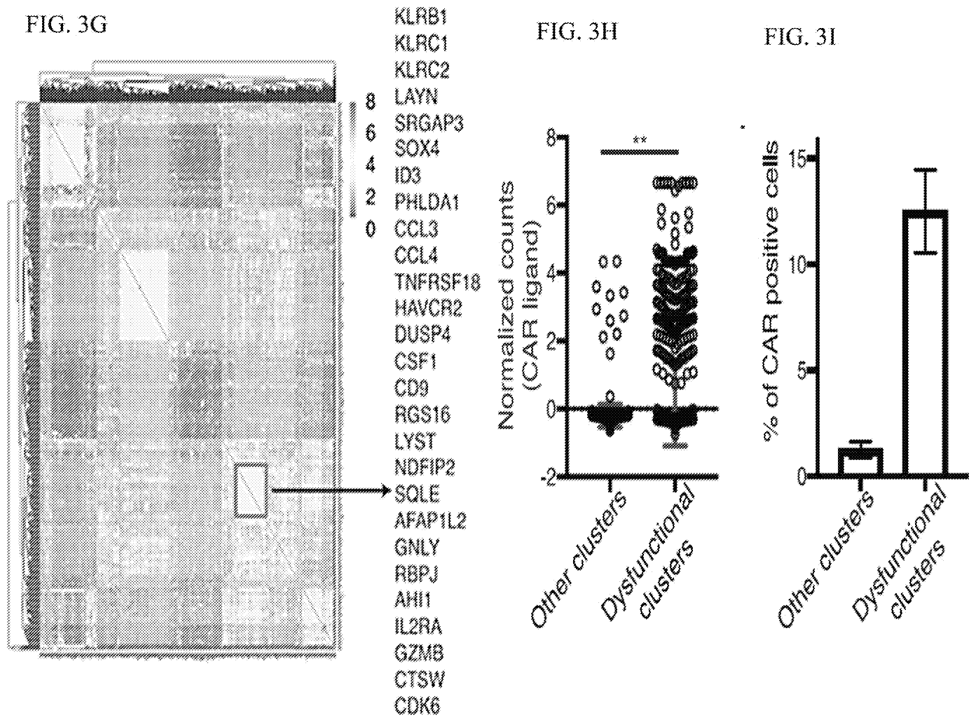

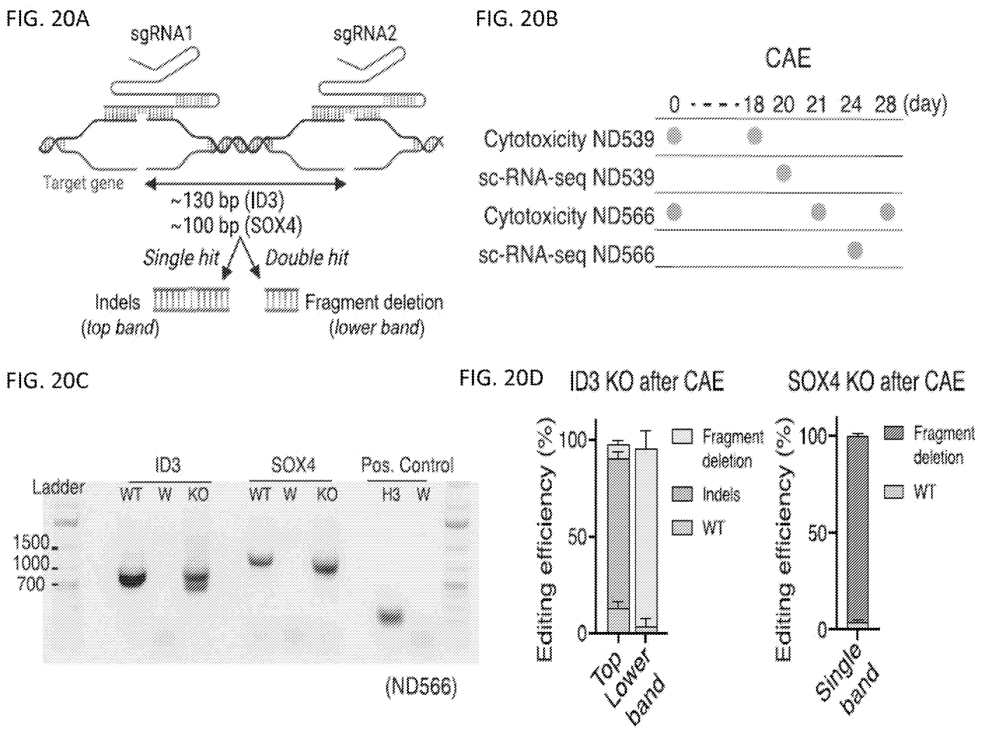

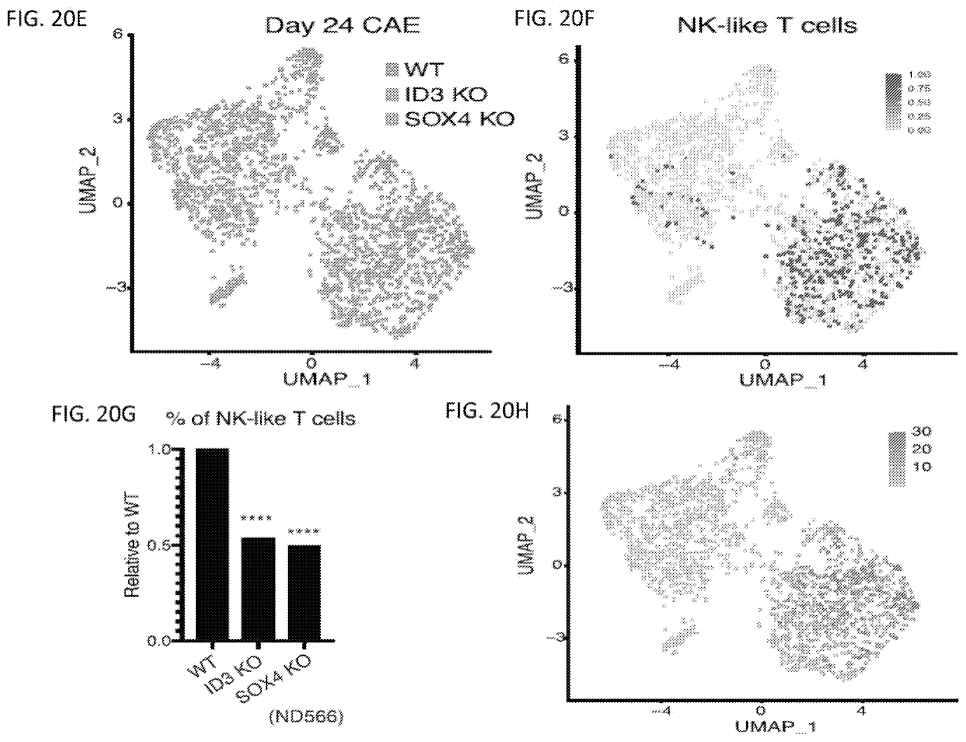

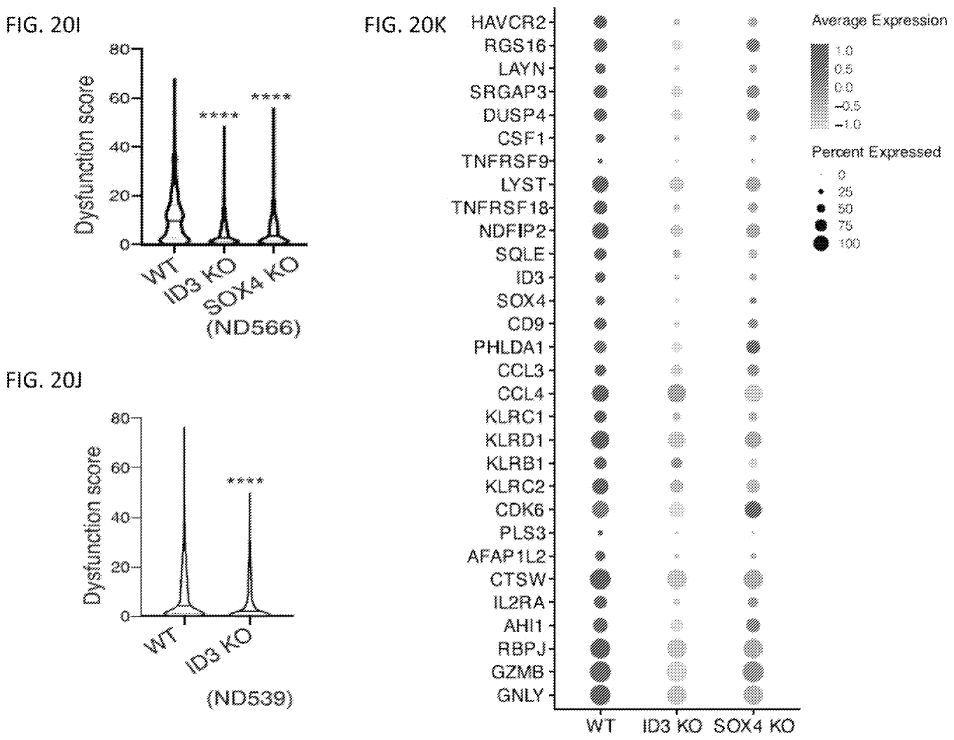

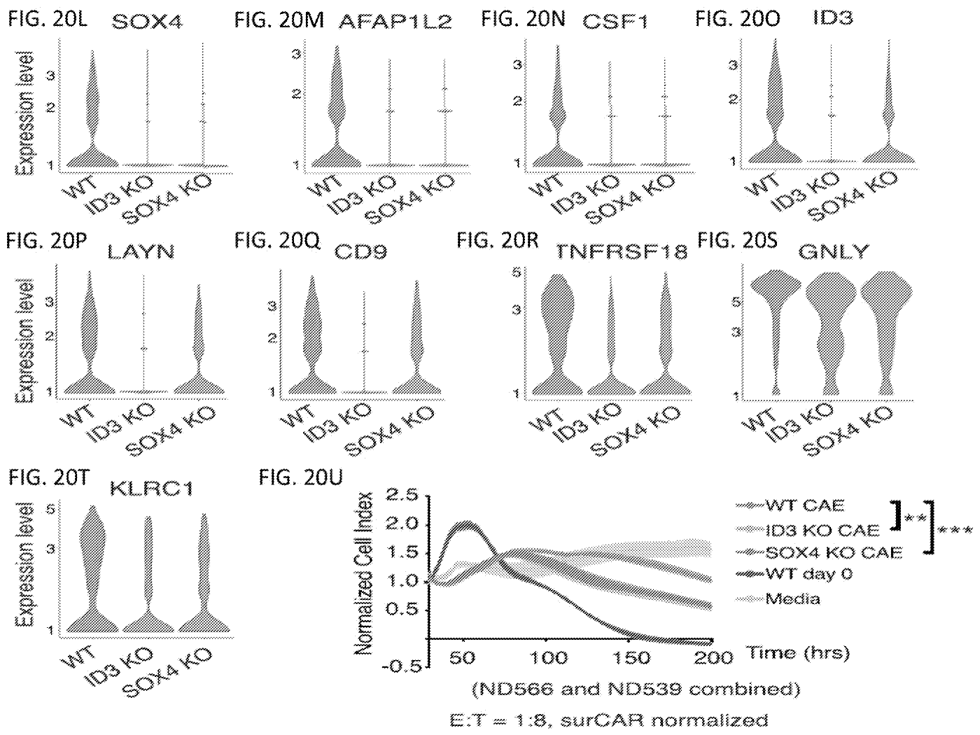

Using polyclonal (bulk) genomics described above, a dysfunction gene signature expressed in cells following CAE was identified. However, it is unclear whether the entire population of T cells express the gene signature or if a subpopulation of CAR T cells dominates in the gene expression. Therefore, single-cell RNA-sequencing (scRNA-seq) was performed for day 0 and day 20 CAE cells. Of note, this experiment was performed in CAR-transduced CD8+ T cells and thus included a mixed population of surCARpos, surCARneg, and untransduced CD8+ T cells. Differentially expressed genes (DEGs) between day 0 and 20 CAE cells were first identified using “cellfishing” (Sato et al., (2019) Genome Biol 20, 31). A strong correlation with findings using polyclonal RNA-seq was found (FIG. 11D). Next, a nonlinear dimensionality-reduction technique (uniform manifold approximation and projection, UMAP) was performed followed by unsupervised clustering on cells from day 0 (FIG. 3A) and 20 (FIG. 3B). The program identified three distinct clusters on day 0 (D0-1, D0-2, D0-3) and four clusters on day 20 (D20-1, D20-2, D20-3, D20-4). Top marker genes (genes that define each cluster) were identified for day 20 CAE (FIG. 3C) and day 0 cell clusters (FIG. 11E). Interestingly, a group of genes upregulated in surCARpos CAE cells identified via polyclonal genomics (FIG. 2A) (KLRC1, SOX4, TNFRSF18, RBPJ, RGS16, CCL3) were found to be top marker genes for single-cell clusters D20-1 and D20-4. Furthermore, gene pathway analysis using all DEGs for each cluster revealed enrichment of the term “natural killer signaling” in day 20 CAE cell clusters D20-1 and D20-4, but not D20-2 and D20-3 clusters or day 0 clusters (FIG. 3D, FIG. 11F). Overlap of the top marker genes for each single-cell cluster revealed that genes defining clusters D20-1 and D20-4 significantly overlapped with genes upregulated in day 28 CAE cells via bulk genomics. Thus, D20-1 and D20-4 clusters likely represent a subpopulation of CAE cells consisting of dysfunctional CD8+ T cells that express NK associated genes. Genes that were downregulated in polyclonal CAE surCARpos T cells and thus highly expressed in day 0 cells (IL7R, LTB, CD48, HLA-DRB1) were top marker genes for clusters D20-2 and D20-3, suggesting that the cells in these clusters have attributes similar to day 0 cells. Of note, clusters D20-1 and D20-3 are highly enriched for cell cycle regulated pathways (see FIG. 3D) and thus FIG. 3B UMAP_1 likely separates cell clusters based on cell cycle genes, while UMAP_2 separates on the dysfunction signature genes.

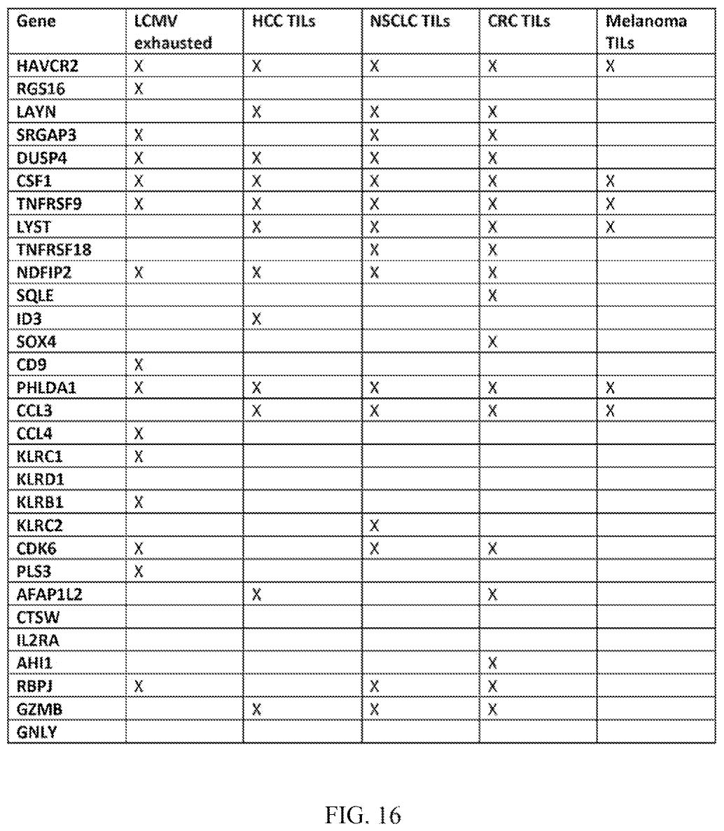

All genes specifically expressed in the presumptive dysfunctional clusters (D20-1 and D20-4), compared to clusters D20-2 and D20-3 (FIG. 3E) were identified. Genes with known links to exhaustion, including HAVCR2, ENTPD1, LAYN, CTLA4, PHLDA1, TNFRSF9, NR4A1, PRDM1, and LAG3 were upregulated in the dysfunctional clusters (FIG. 3E volcano plot, right side). An unbiased dysfunction gene signature was curated consisting of the top 30 genes most highly upregulated in day 20 dysfunctional clusters (log₂FC>0.64, padj<1.0e22) (FIG. 3F), of which 24/30 genes were also upregulated in polyclonal CAE surCARpos T cells (FIG. 2A). Single-cell analysis identified genes that were not within the polyclonal RNA-seq signature, likely due to increased sensitivity of isolating the dysfunctional subpopulation of cells. These newly identified genes included SRGAP3, DUSP4, CSF1-genes not currently linked to T cell exhaustion. Clusters that emerged that were not dysfunctional (D20-2,D20-3) highly expressed HLA molecules (HLA-DRB1, HLA-DQB1, HLA-DRA, HLA-DPB1) and IL7R, TC2N, and FYB1 (see FIG. 3E, left side). In summary, single-cell analysis led to the identification of a dysfunction signature comprised of 30 genes (FIG. 16).

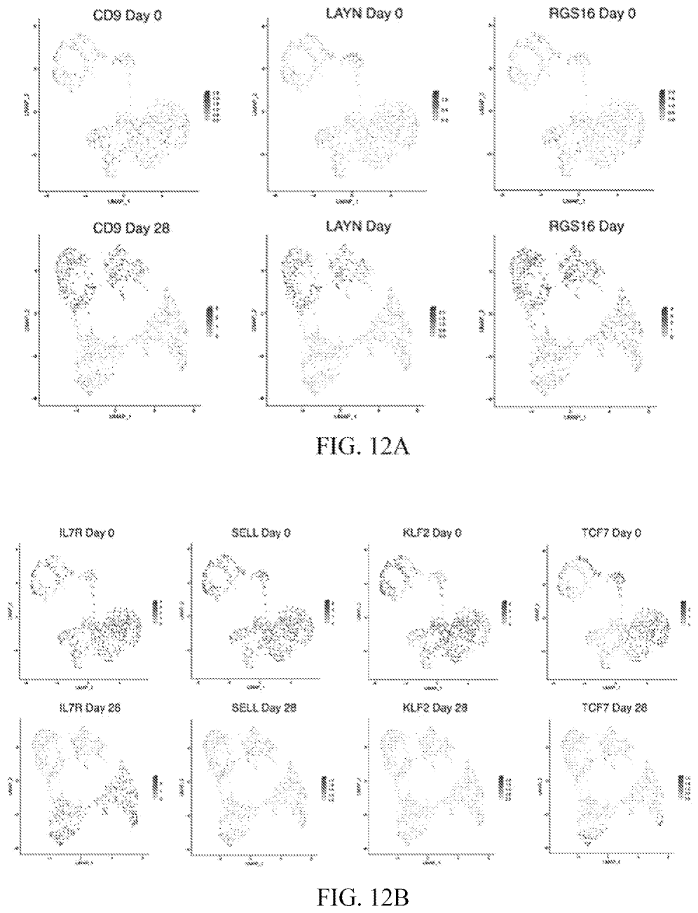

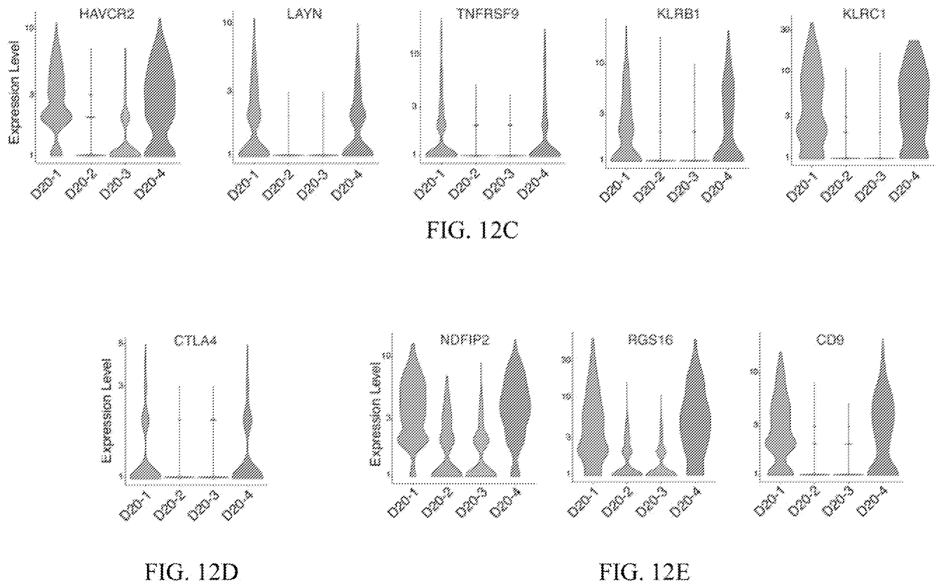

To illustrate differences in the dysfunction signature between day 0 and day 20 CAE cells, dot plots containing the 30 signature genes, as well as naïve/memory markers (IL7R, TCF7, 5 SELL, KLF2), cell cycle genes (TUBA1B, TOP2A, PCNA), and control genes (CD8A, CD3E) were generated (FIG. 3F). Of note, many of the dysfunction signature genes are also present in the gene expression signature described for other models of T cell dysfunction (FIG. 16). Day 20 CAE cells (FIG. 3F, right) had two cell clusters that highly express the dysfunction signature (clusters D20-1, D20-4), while clusters D20-2 and D20-3 and day 0 cell clusters (FIG. 3F, left) did not express this signature (also see UMAPs, FIG. 12A). Day 0 clusters and D20-2 and D20-3 clusters expressed IL7R while TCF7, SELL and KLF2 (naïve markers) were specific to day 0 clusters (also see UMAPs, FIG. 12B). Violin plots of genes with known links to T cell exhaustion/dysfunction (HAVCR2, LAYN, TNFRSF9) and NK receptor genes (KLRB1, KLRC1) showed specific expression in D20-1 and D20-4 clusters (FIG. 12C). Although not part of the 30 gene signature, CTLA4 (an immune checkpoint molecule associated with T cell exhaustion) was upregulated in D20-1 and D20-4 clusters (padj 1.2e-11, log₂FC 0.20) (FIG. 12D). Taken together, these observations demonstrated that 20 days of CAE results in the upregulation of a gene expression signature that includes NK receptor and exhaustion markers, as well as candidate genes without previous association with T cell dysfunction (SRGAP3, RGS16, DUSP4, NDFIP2, CD9) (FIGS. 11G and 12E). Select T cell activation genes identified in CD19 CAR T cells (CCL3, CCL4, GZMB, TNFRSF9) are in the 30-gene signature; however, many inhibitory receptors are also T cell activation genes, and their sustained expression is a hallmark feature of T cell exhaustion (Wherry and Kurachi, (2015) Nat. Rev. Immunol. 15, 486-499).

It was investigated whether the dysfunction signature genes are co-expressed within the same single cell. To address this question, an unbiased gene regulatory network analysis (PIDC) was implemented to identify genes that are co-expressed or co-regulated in day 20 CAE cells (Chan et al., (2017) Cell Syst 5, 251-267.e253). One community included 34 genes that are co-expressed (FIG. 3G; boxed). Strikingly, 27/30 of the defined dysfunction signature genes (FIG. 3F) are contained within this 34 gene community, confirming that these genes are co-expressed in the same subset of cells and demonstrating that they have a common regulatory network (FIG. 3G). Genes that are co-expressed with the dysfunction genes but are not within the dysfunction signature included APOBEC3G, IKZF3, BHLHE40, CCND2, NAMPT, SAMSN1, and PIP5K1B.

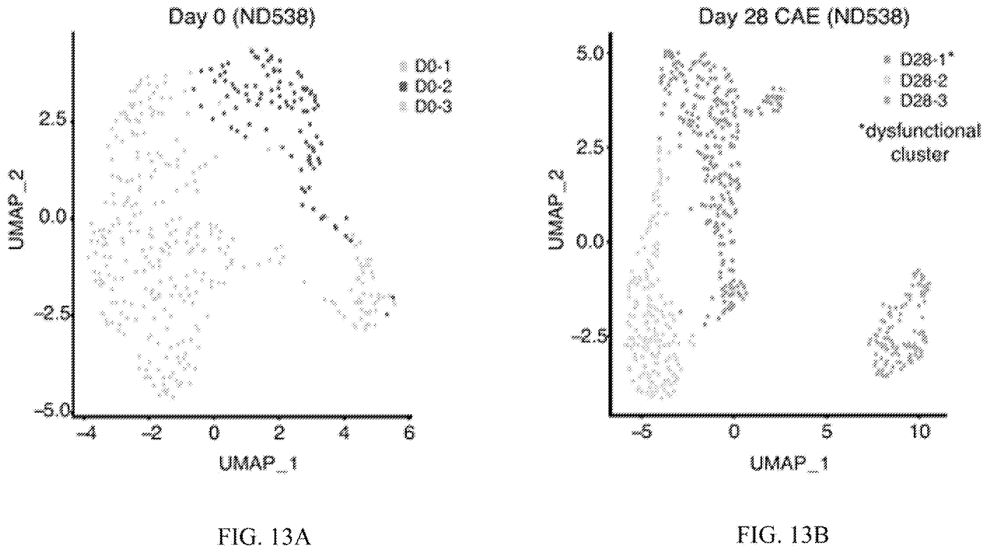

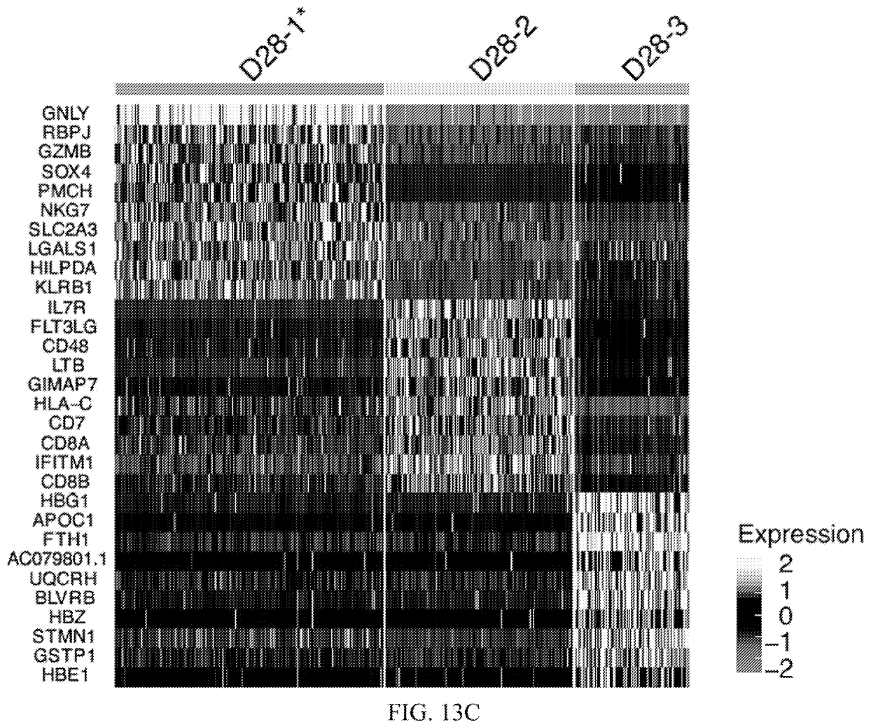

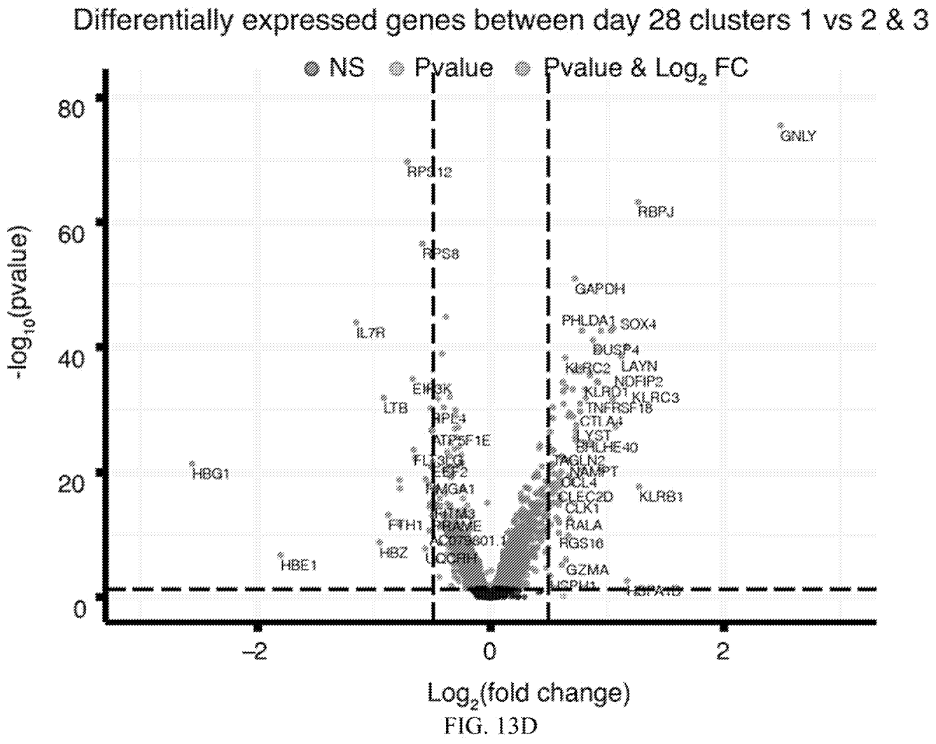

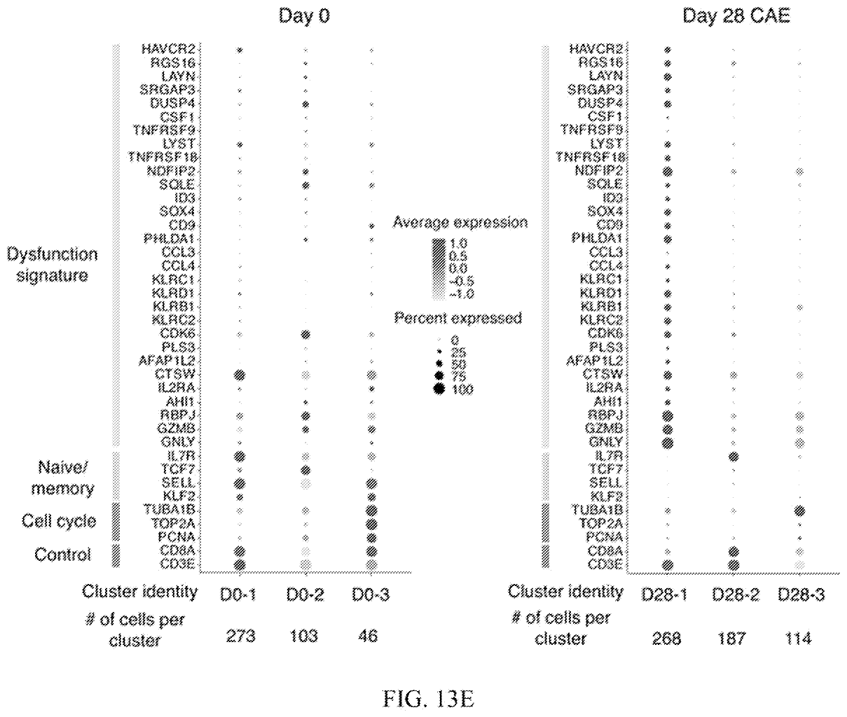

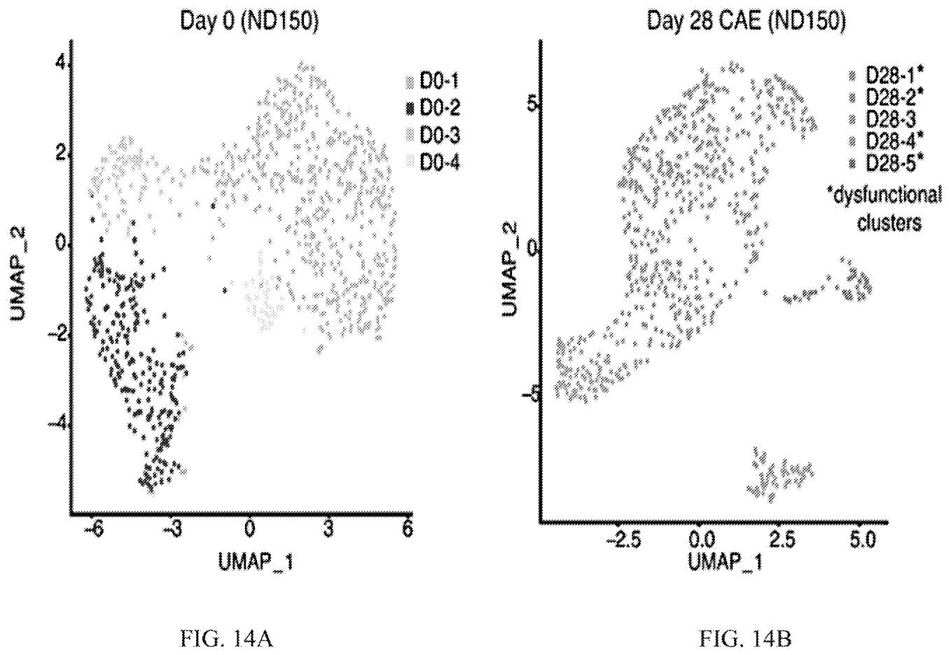

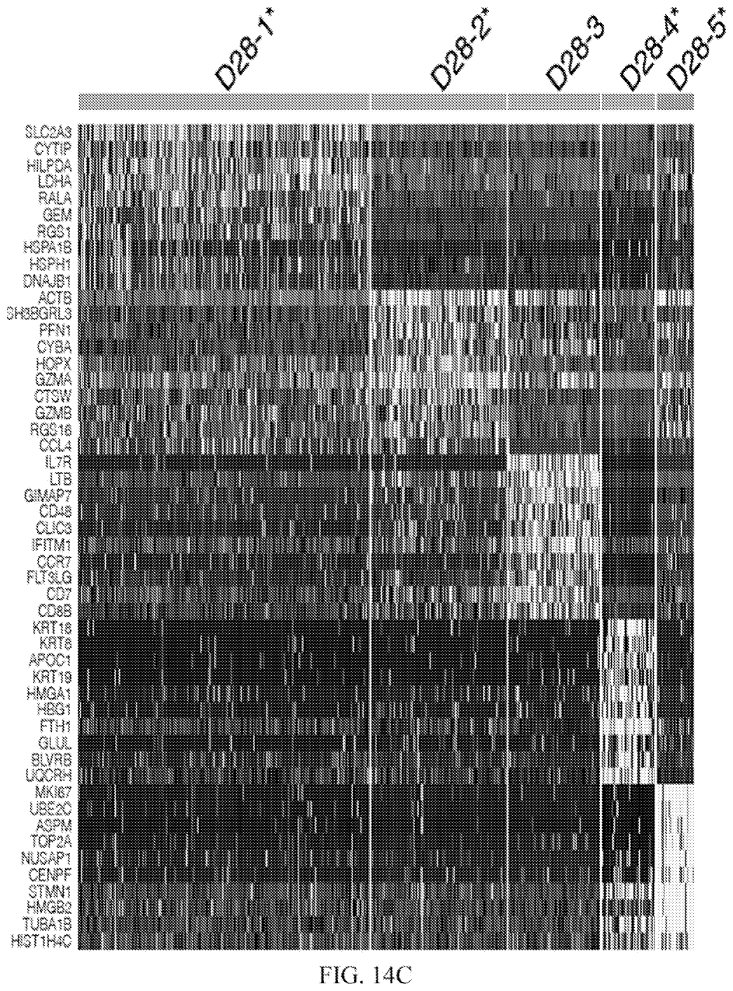

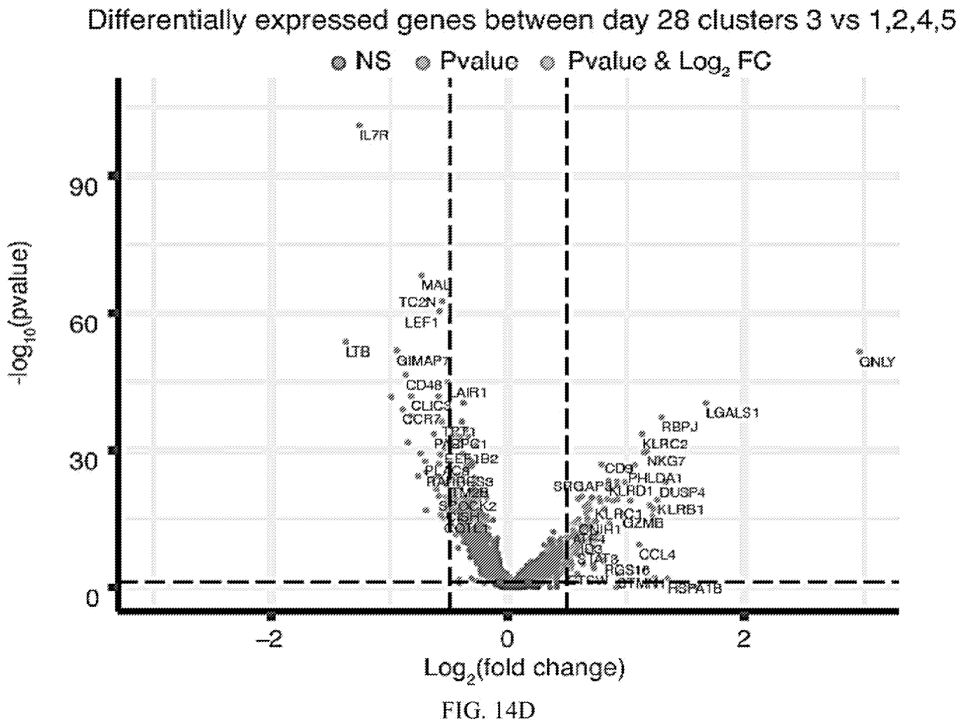

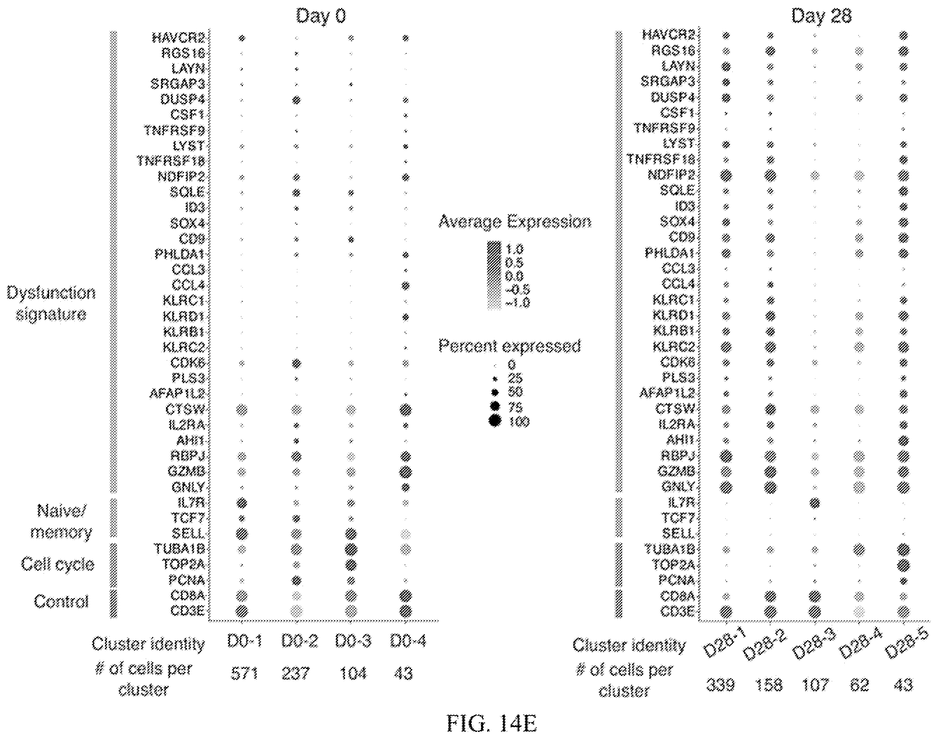

Importantly, to confirm the single-cell findings, scRNA-seq was performed in two additional donors (ND538 and ND150) for day 0 and 28 CAE cells. Remarkably similar gene expression signatures were found, despite these cells being collected at later timepoints of CAE (FIGS. 13 and 14). Human donors have variability in the number of days required to reach a dysfunctional state; however, most CAR T donors are dysfunctional by 20 days of CAE.

Given that CAE results in dysfunctional CAR T cells with reduced effector function, it was next asked whether CAR transcripts could be detected in the single-cell datasets, and if so, whether cells that express the CAR are preferentially expressed in the dysfunctional cell clusters. The hypothesis was that continuous stimulation through the CAR drives the dysfunction signature. Cells from all three single-cell experiments (ND388 day 20 CAE cells, ND538 and ND150 day 28 CAE cells) were combined and the dysfunctional cell clusters (defined for each donor separately, see FIGS. 3, 13, and 14) expressed significantly more CAR (FIG. 3H) and had a higher percentage of cells overall that expressed the CAR (FIG. 3I). Notably, these data provided a functional link between the dysfunction cell and gene signature and diminished CAR T cell killing.

In summary, a robust gene expression signature was identified that defines dysfunctional CAR T cells and is associated with reduced CAR T cell killing; this signature has implications for the effectiveness of CAR T cell therapy in solid tumors. The CAR T cell dysfunction signature, including upregulation of exhaustion genes, and, notably, upregulation of NK receptors on CD8+ T cells, suggests that these cells may have acquired an NK-like phenotype.

### Example 5: Mass and Flow Cytometry Profiling Reveals NK-Like Phenotype of CD8+ CAR T Cells Under CAE

A dysfunctional gene expression signature of CAR T cells was uncovered that includes the upregulation of many NK receptor genes by polyclonal and scRNA-seq. However, expression of mRNA is not always predictive of protein expression, therefore, expression of NK-associated proteins was examined by flow cytometry on surCARpos and surCARneg CD8+ T cells. In alignment with the polyclonal and scRNA-seq data, upregulation of CD94, NKG2A, and KLRB1 protein occurred in prolonged CAE (FIG. 4A, top). Increased expression of both CD56 and PD-1 also was detected at the protein level (FIG. 4A, bottom), but not by scRNA-seq-possibly due to the low expression levels of these genes. CD8+ CAR T cells did not express high levels of NK-associated molecules before CAE, but exhibited increased expression of NK-markers with different time courses after CAE (FIG. 4A). The frequency of CD94+ T cells (KLRD1) increased immediately after one stimulation and remained high throughout CAE. This result suggests that expression of CD94 is specific to signaling through the CAR since day 0 CAR T cells have been stimulated with CD3 and CD28 beads, but do not express CD94. This notion is further supported by the delay in expression of CD94 in surCARneg verses surCARpos T cells (FIG. 4A). NKG2C expression levels initially rose in a pattern similar to CD94, and then showed approximately a 75% decline in percent NKG2C positive cells around day 15 (FIG. 4A). In contrast, expression levels of NKG2A, TIGIT, and CD56 steadily increased over time. The activating receptor NKG2C and the inhibitory receptor NKG2A both bind to CD94 to toggle NK cell function. The timing of expression of NKG2C and NKG2A in CAE M5CAR T cells suggests that CD94 initially binds NKG2C and later NKG2A outcompetes for ligand engagement to override NKG2C-induced effector function. Interestingly, CD94 can also function as a homodimer. KLRB1 was expressed in the late phase of the study. Levels of CD28 decreased with CAE in both surCARpos and surCARneg populations (FIG. 4A). Interestingly NK receptors are thought to provide alternative costimulatory pathways for innate-like T cells. PD-1 expression dramatically increased in surCARpos CD8+ T cells after one stimulation and then PD-1 expression was maintained at a moderate level after CAE (FIG. 4A). surCARneg CD8+ T cells showed higher expression of CD56 than surCARpos CD8+ T cells (FIG. 4A, bottom), emphasizing that both loss of surface CAR expression, and CD56 expression are associated with the dysfunctional CAR T cell state. Importantly, invariant NKT cells (iNKT, defined as cells with Va24-Ja18 specific TCRs) were not identified over the time course of the study (FIG. 4A, bottom), suggesting NK-like T cells identified in this model need to be separately classified from iNKT cells.

Cytometry by time-of-flight (CyTOF or mass cytometry) was performed in addition to using the flow cytometry data to explore how the dysfunction signature identified by scRNA-seq relates to protein expression levels on CAR T cells. T-distributed stochastic neighbor embedding (t-SNE) plots of 29 NK-associated molecules were generated to visualize the phenotypical differences between day 0 product and day 29 CAE CAR T cells (FIG. 4B). Twenty subpopulations of CD8+ T cells were identified. Strikingly consistent with sc-RNA-seq observations, CAE CAR T cells had markedly different clusters compared to day 0 product (FIG. 4B, circle denotes cell populations more abundant in CAE T cells). Notably many NK receptors and NK-related proteins were increased in the CAE specific clusters, including the inhibitory receptors (KLRB1, TIGIT, NKG2A, PD-1) and NK-related proteins CD56 and Granulysin (FIG. 4C). These mass cytometry data closely aligned with the flow cytometry profiling of CD8+ CAR T cells under CAE as shown in FIG. 4A, however, the peak of NKG2C expression seen by flow time course analyses was not captured in the mass cytometry data from day 29. The various subpopulations identified in the CAE cells revealed the NK-like phenotype is heterogeneous. Clusters 6 and 11 were predominantly KLRB1+ cells and clustered furthest from the day 0 subpopulations. Furthermore, there were two distinct subpopulations of cells that expressed CD56, one group that was KLRB1+ and another group that was KLRB1−. The significance of the NK-like T cell subpopulations (CD56+KLRB1+, CD56-KLRB1+, CD56+KLRB1−) is an active area of investigation. In agreement with the genomics data (see FIGS. 2B, 10H and 10I), NK-like phenotypes emerged in both surCARpos and surCARneg cells (FIGS. 4A and 4C). However, as M5CAR T cells acquired the dysfunctional phenotype and loss of surface CAR, the activating NK receptor, NKG2C, was no longer expressed. Overall, these data suggest that day 0 CD8+ T cells dynamically evolve into NK-like T cells with a distinct phenotype marked by KLRB1 and/or CD56 expression.

### Example 6: In Vivo NK Receptor Upregulation and Dysfunction Signature Gene Expression in CAR T Cells and TILs

Observations above of upregulation of NK molecules on CD8+ CAR T cells in vitro during CAE prompted testing of whether this expansion occurs in vivo. AsPC-1 tumors were established in mice and M5CAR T cells were able to eliminate large mesothelin-expressing flank tumors within 2 weeks after CAR T injection (FIGS. 4D and 4E). However, 2 to 4 months after initial injection of the CAR T cells, several of the mice relapsed. The recurrent tumors were analyzed and it was discovered that the mechanism of tumor relapse was not due to loss of the mesothelin target antigen. Therefore, the infiltrating human T cells in the relapsed tumors were analyzed and nearly all the infiltrating T cells were CD8+ CAR T cells. Intriguingly, the CAR T cells from the recurrent tumors expressed the dysfunction signature with high levels of NK receptors (FIGS. 4F and 4G) and checkpoint receptors (FIGS. 4H and 4I), unlike the day 0 CAR T product. Further, since the tumors were progressing without losing mesothelin expression, it can be concluded that the T cells had lost the ability to control the tumor and are thus dysfunctional.

This finding prompted testing of whether this expansion occurs in patients undergoing CAR T therapy. Diffuse large Bcell lymphoma (DLBCL) patients treated with CD19-directed CAR T cells (CTL019) were retrospectively assessed in a clinical trial (NCT02030834) to determine whether any of their circulating CAR T cells exhibited NK-like features. Three of seventeen analyzed DLBCL patients exhibited greater than 5% expansion of the CAR+ NK-like T cell population as early as 10 days post-CAR T infusion of a CD19-directed CAR, and other patients showed detectable expansion (FIG. 4J). Notably, the patient with the highest level of NK-like CAR T cells (13413-39) had progressive tumor and failed to respond to the therapy (Schuster et al., 2017, N. Engl. J. Med. 377, 2545-2554). There was sufficient material from patient 13413-39 to analyze additional NK markers in CAR+ T cells. The percentage of NK-like T cells in the day 0 CAR T product was low, but the NK-like CD8+ T cell phenotype was upregulated at day 27 post-CAR T infusion as determined by increased levels of NKG2A, CD94, and CD56 (FIG. 4K). Increased KLRB1 levels were not detected; however, this could be explained by the late expression of this marker upon CAE (FIG. 4A). In conclusion, these data provide evidence for the acquisition of an NK-like CAR T cell phenotype in some CAR T cell patients.

To determine whether the CAR T dysfunction signature is CAR-specific or more broadly applicable to T cells chronically exposed to antigen, lung tumors that expressed the antigen NY-ESO-1 in a xenograft mouse model were generated, and then human T cells specifically engineered to express NYESO-1-reactive Ly95 TCR were injected into the tumor (FIG. 4L). This generates hypofunctional Ly95 TILs that are unable to eradicate tumor (Moon et al., (2016) Clin. Cancer Res. 22, 436-447). The dysfunction gene signature was expressed at a low level in the infused product and blood CD8+ T cells, but strikingly, 28/30 of the exhaustion and NK signature genes were upregulated in the NY-ESO-1-reactive TCR TILs, including the transcription factors ID3 and SOX4 (FIG. 4M).

### Example 7: Transition of CD8+ T Cells to NK-Like T Cells Upon Continuous Antigen Stimulation

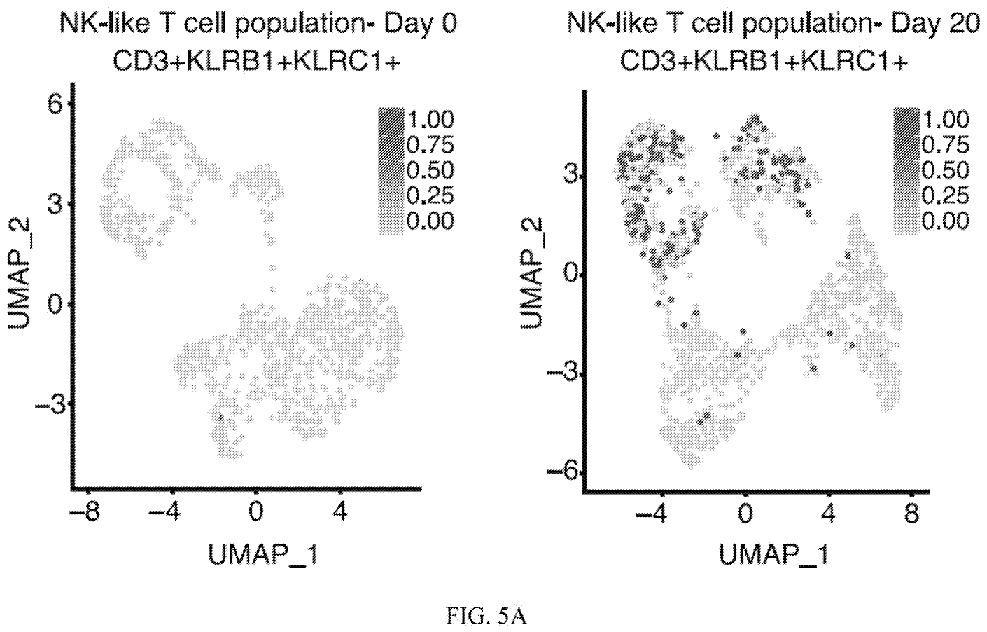

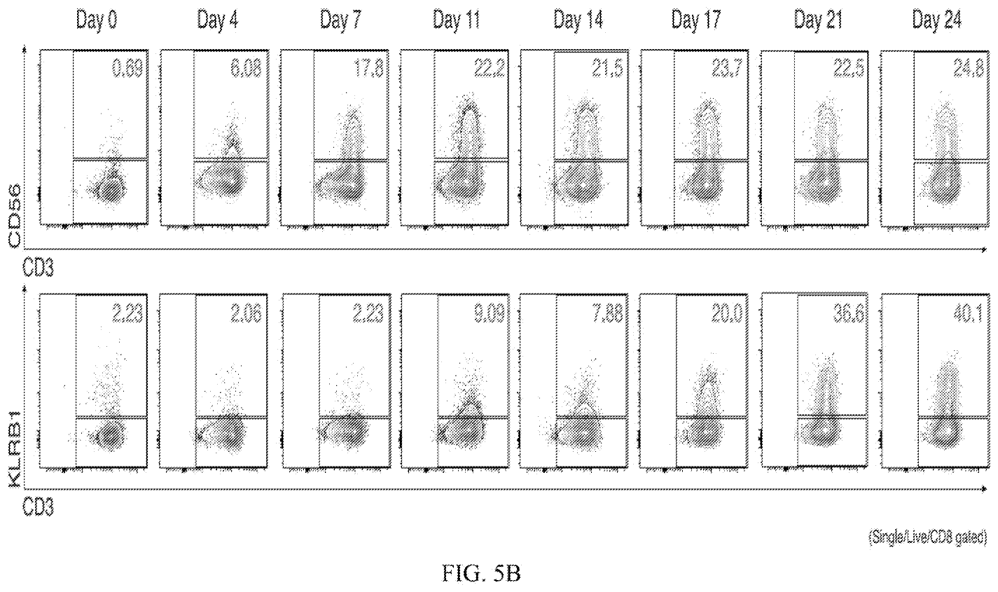

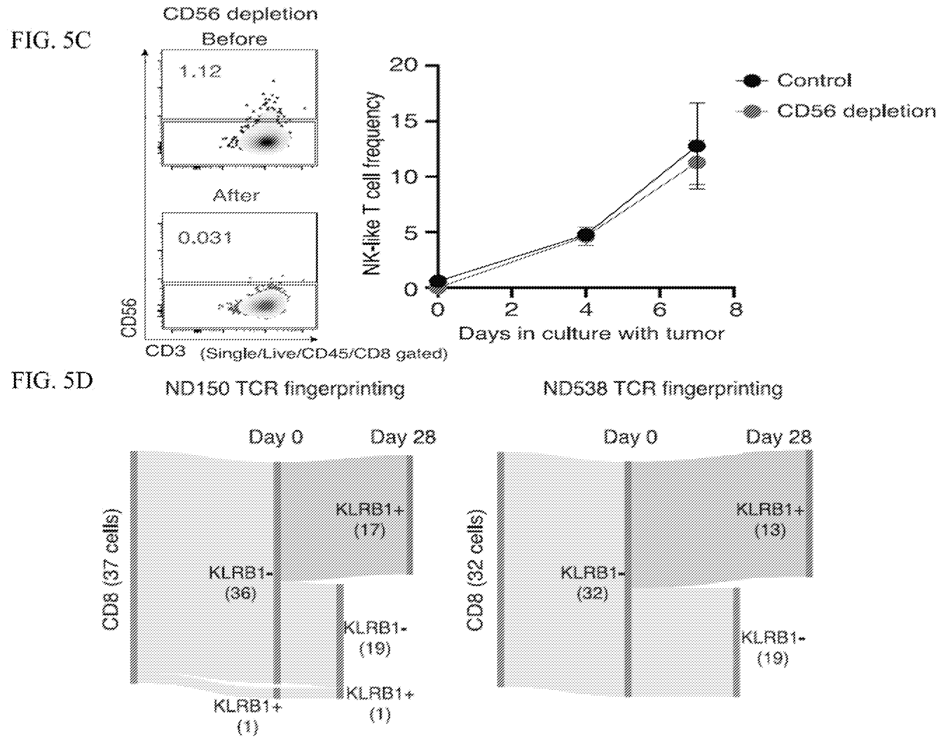

NK-like T cells have been shown to express both T cell and NK cell markers and are frequently defined as CD3+CD56+ or CD3+KLRB1+ and they often express KLRC1. UMAP plots of scRNA-seq day 0 vs. day 20 CAE cells showed enrichment of cells that co-express CD3, KLRB1, and KLRC1 (FIG. 5A, related to UMAPs in FIGS. 3A-3B). In addition, flow cytometry analysis using two separate markers for NK-like T cells (CD3+CD56+ and CD3+KLRB1+) revealed a robust expansion of this NK-like T cell population during the course of CAE (FIG. 5B).

These findings overall demonstrate expansion of an NK-like T cell population upon CAE; however, it is unclear whether these are clonally expanded cells from an NK-like T population existing at day 0, or, in contrast, whether CD8+ T cells acquire NK receptors during prolonged antigen exposure. To test this in the in vitro model of CAR T cell dysfunction, CD56+ cells were depleted from the input day 0 population using anti-CD56 coated beads and the CAE experiment was repeated. CD56 is the most frequently used marker to identify human NK and NK-like T cells and hence CD56 depletion was expected to remove both populations from the day 0 product. At day 0, the percentage of NK-like T cells was very low (0.69-2.23%, FIGS. 5A and 5B left). The prediction was that if pre-existing NK-like T cells at day 0 expand, depletion of CD56⁺ cells will result in reduced NK-like T cells upon CAE, whereas if instead there is a transition from CD8+ to NK-like T cells, CD56⁺ depletion will not reduce the number of NK-like T cells (see theoretical model, FIG. 15A). Strikingly, CD56⁺ depletion had no effect on the percent of NK-like T cells that emerged upon CAE (FIG. 5C, right), suggesting transition of T cells to NK-like T cells rather than expansion.

To confirm the T cell to NK-like T cell transition, scRNA-seq was performed alongside lineage tracing using T cell receptor (TCR) sequencing in two donors (ND150 and ND538) at day 0 and 28 of CAE (FIG. 5D). It was reasoned that the specific TCR allele would be the same after transition. CD8+ cells with TCRs in common between day 0 and 28 were filtered (N=37 cells for donor ND150, FIG. 5D, left). Of these, 36 were KLRB1− at day 0 and by day 28, 17/36 (47%) transitioned to KLRB1+, clear evidence of a transition because the KLRB1− cell at day 0 had the same TCR sequence as the KLRB1+ cell at day 28. This was validated independently using donor ND538 cells (FIG. 5D, right). These results confirmed that the NK-like T cells are undergoing transition, and not simply expanding. 96-99% of the TCRs were unique in each sample, providing additional evidence against clonal expansion in the in vitro model (FIG. 15B).

To model the changes in transcription that occur as CD8+ T cells transition to NK-like T cells, pseudotime analysis was performed using the Monocle single-cell software. Pseudotime is a quantitative measure of biological progression through a process, such as cell differentiation, that allows users to order cells and track transcriptional changes that occur during that process (Qiu et al., (2017) Nat Methods 14, 309-315; Trapnell et al., (2014) Nat Biotechnol 32, 381-386). Pseudotime analysis showed that day 20 CAE clusters (D20-2, D20-3) separate from dysfunctional clusters (D20-1, D20-4), with transcriptional progression from D20-3, D20-2, D20-4 to D20-1 (FIG. 5E, left). Consistent with this progression, cells expressing the dysfunction signature (see FIG. 3F, N=30 genes) prominently occupy the end of the trajectory (FIG. 5E, right). Two additional donors were used to validate these findings (ND150 and ND538) and importantly, day 0 and 28 CAE samples from both donors were combined together for pseudotime analysis. Pseudotime analysis revealed that day 0 samples cluster together on the right side of the trajectory, while day 28 samples cluster together on the left (FIG. 5F, left). Furthermore, cells expressing the highest level of dysfunction signature genes cluster on the left side of the trajectory with day 28 CAE cells (FIG. 5F, right). Taken together, the dysfunction signature genes associate with transitioned NK-like T cells and the transcriptional changes that occur during this progression can be successfully mapped.

### Example 8: ID3 and SOX4 are Regulators of the Dysfunction Signature

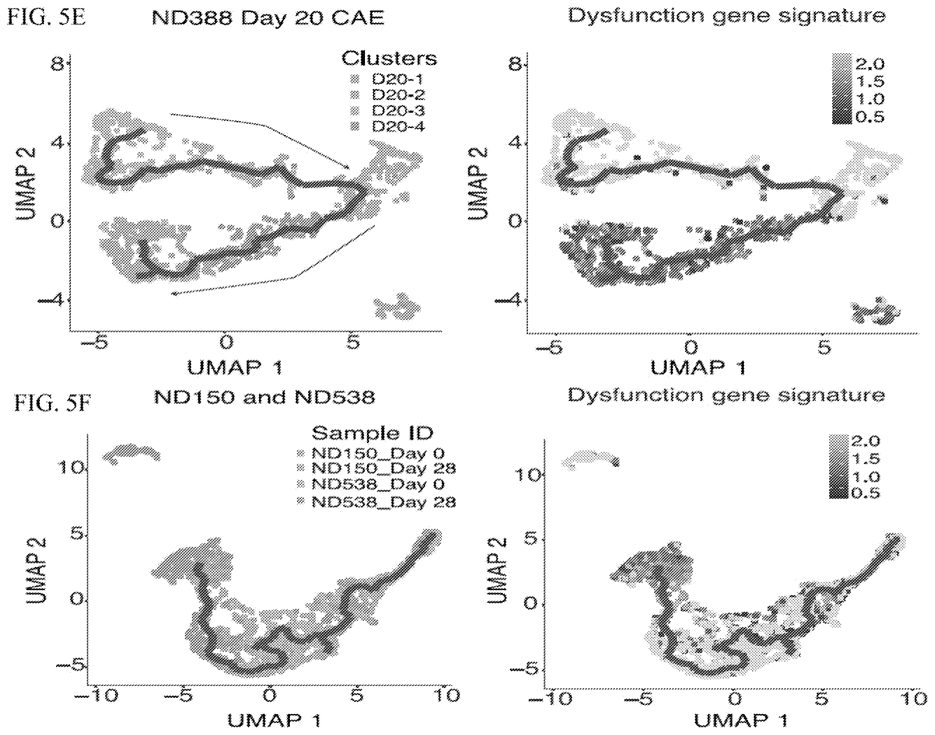

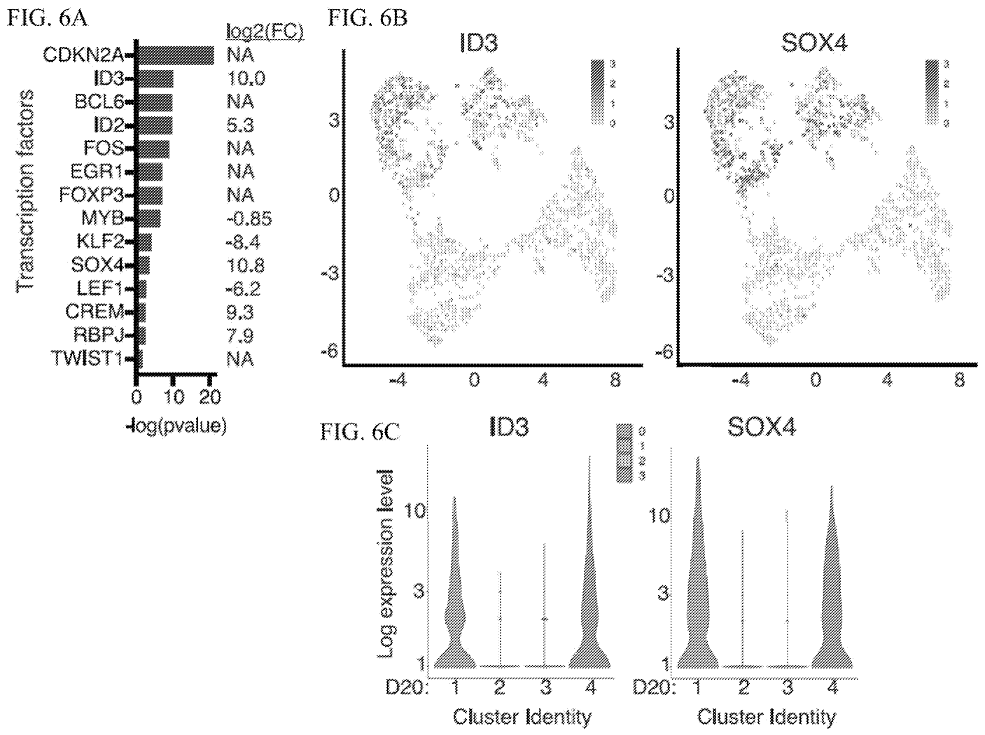

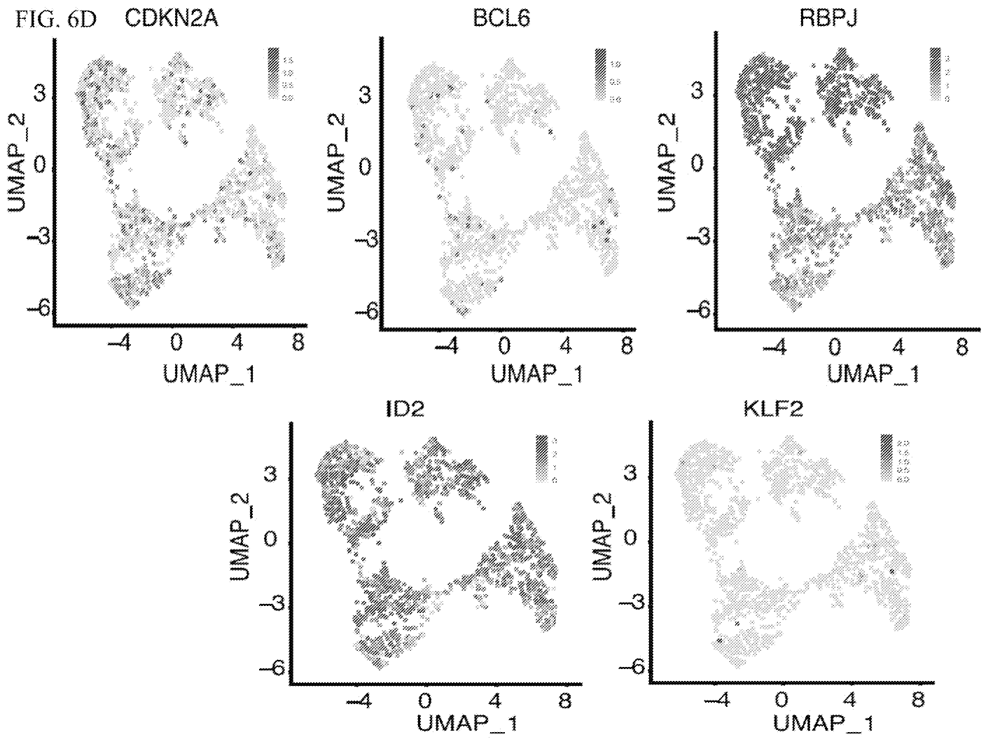

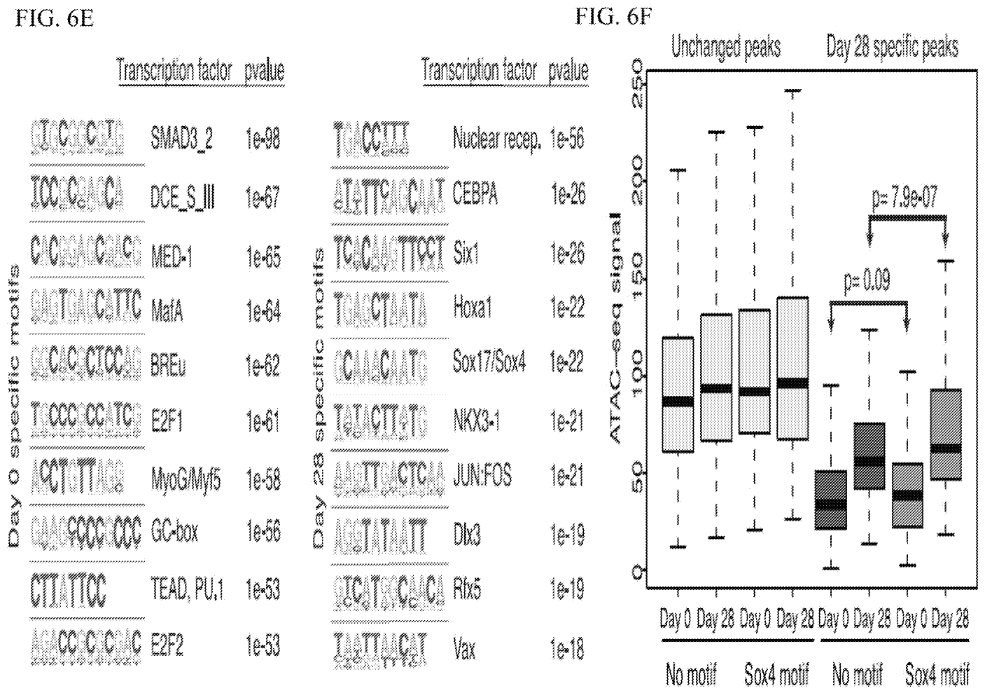

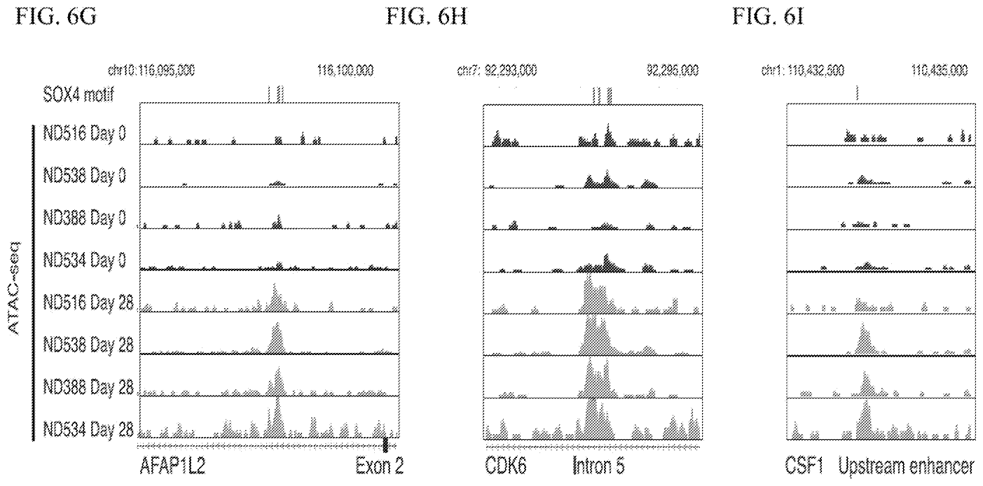

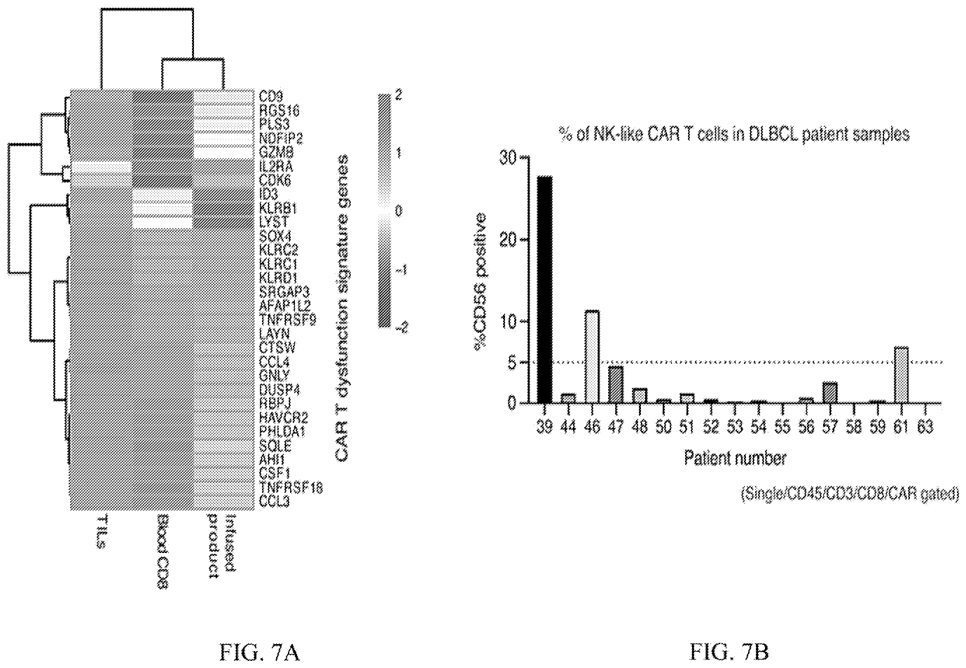

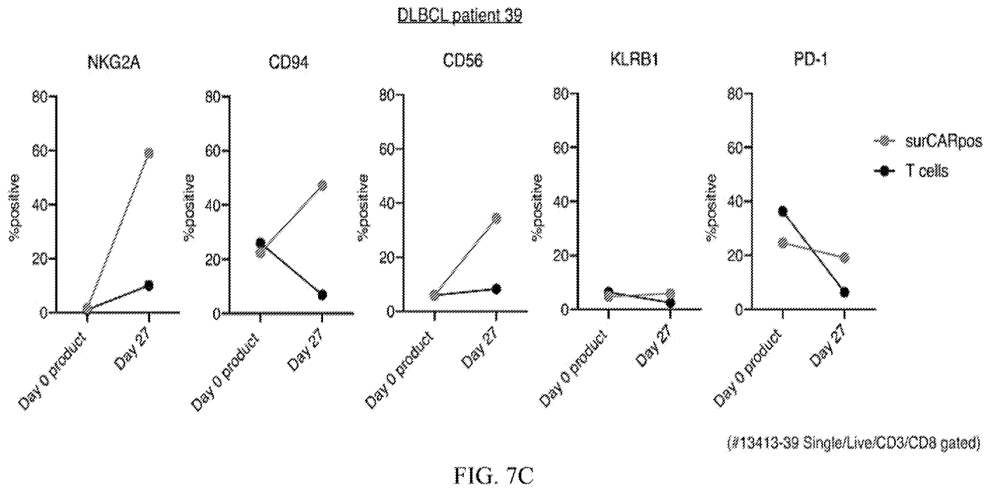

Identification of a common transcription factor(s) that controls this novel CAR T dysfunction signature and NK-like T cell transition could provide an approach to prevent and/or reverse loss of effector function. Regulatory network analysis (FIG. 3G) and monocle trajectory analysis (FIGS. 5E and 5F) of the CAE cells suggest that the dysfunction signature genes may be under the control and/or regulated by a common set of transcription factors. DEGs identified in the scRNA-seq datasets between day 0 and 20 CAE cells were analyzed by IPA to identify potential transcription factors that regulate the signature. All transcription factors highlighted in the polyclonal RNA-seq experiment (FIG. 2F) were also regulators of the single-cell signature and some, but not all, were themselves differentially expressed in the single-cell dataset (FC indicated to the right, FIG. 6A). Next, it was examined whether any transcription factors displayed specific expression in the dysfunctional cell clusters (D20-1 and D20-4, from FIG. 3B). Importantly, ID3 and SOX4 were specifically expressed in the dysfunction clusters (FIGS. 6B-6C), while all other transcription factors, with the possible exception of TWIST1 that was expressed at low levels, lacked specificity or had less dramatic changes between dysfunctional and non-dysfunctional clusters (FIG. 6D). Consistently, ID3 and SOX4 were co-expressed with the other dysfunction signature genes in CAE T cells (see FIG. 3G), suggesting these transcription factors may help to orchestrate the dysregulated gene expression signature.

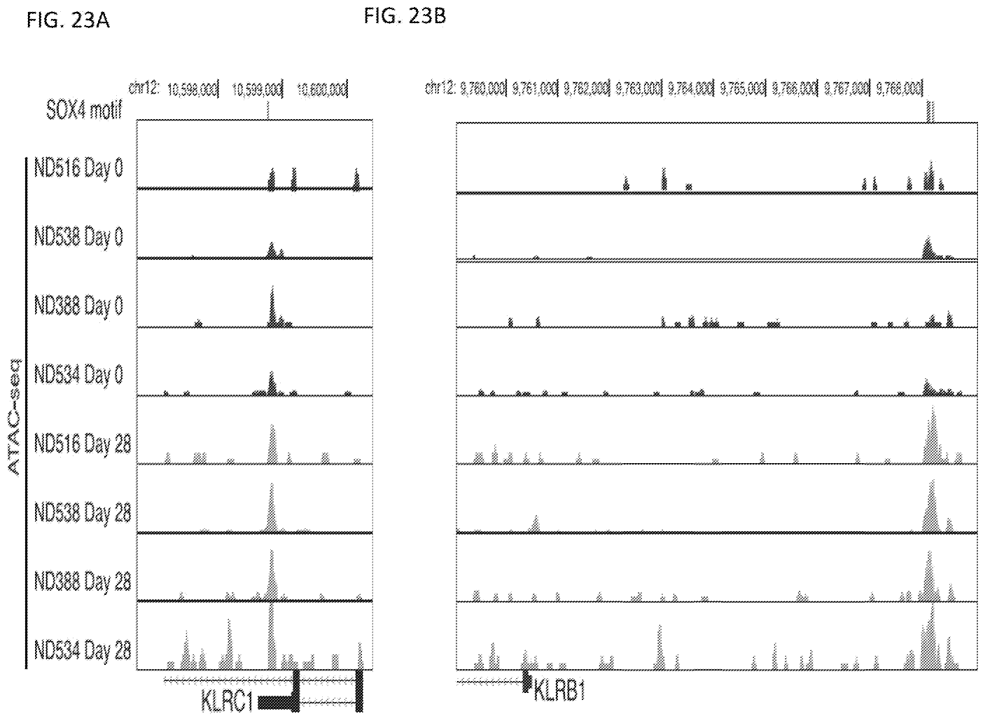

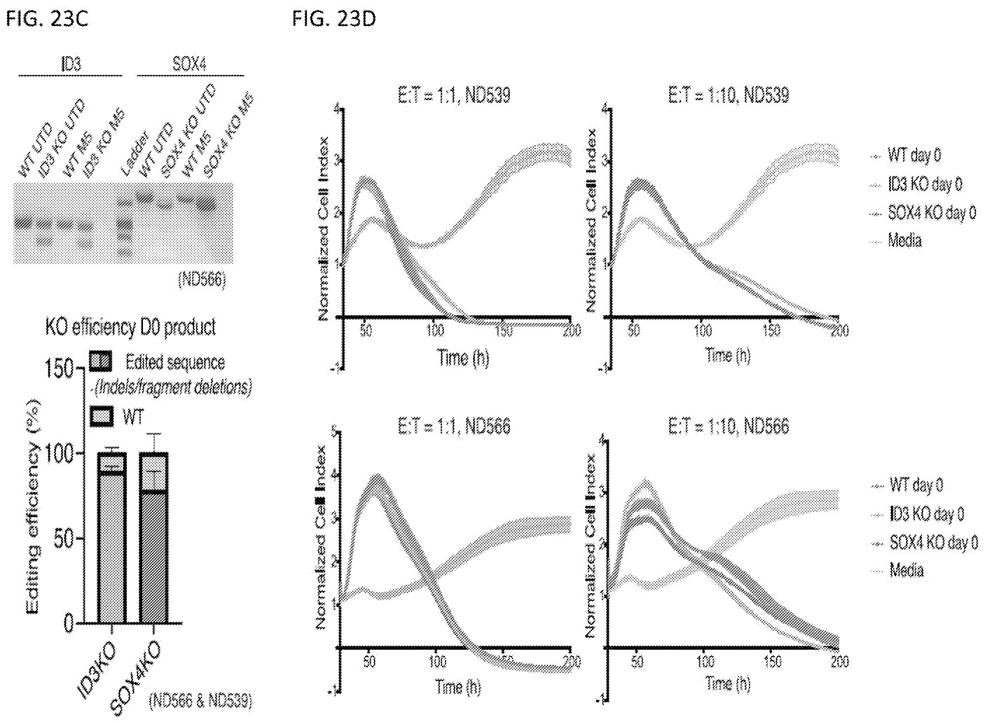

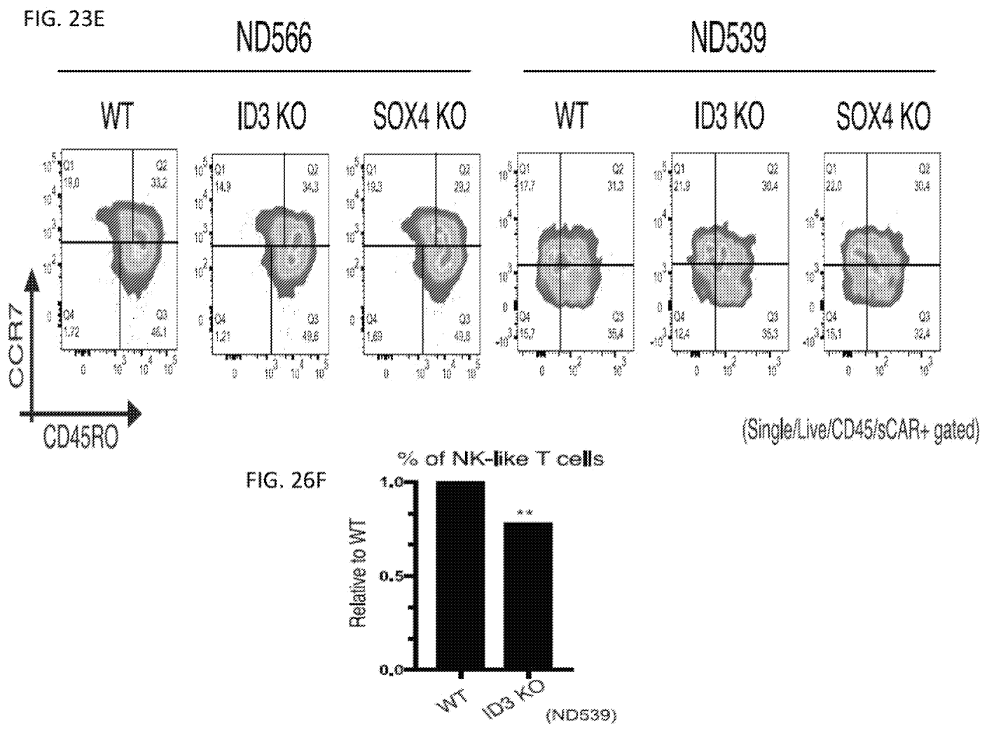

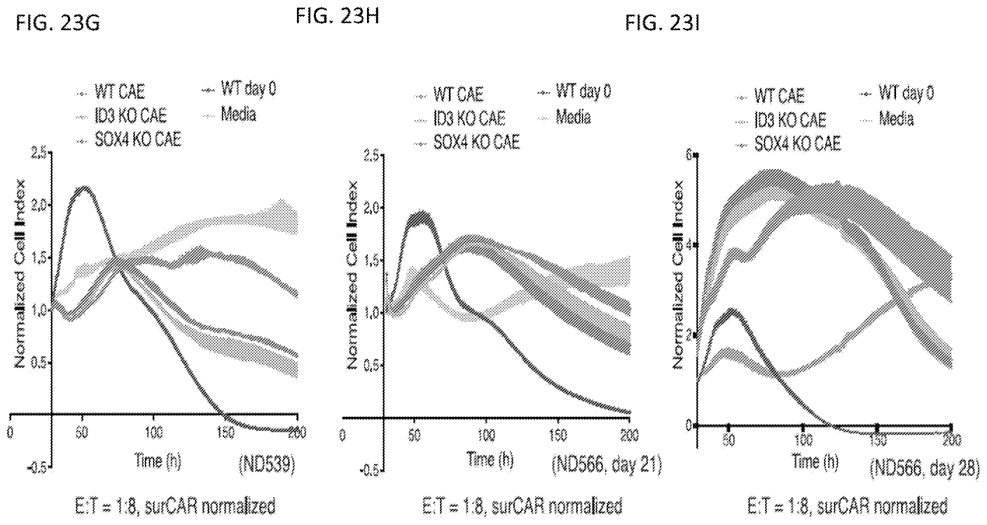

ID3 is a member of a family of helix-loop-helix transcription factors that do not bind DNA directly, but rather inhibit other proteins from binding DNA, and thus, ID3 lacks a specific DNA-binding motif. However, SOX4, a member of the SRY-related HMG-box family, has a known DNA motif. Unbiased motif enrichment analysis (HOMER) was used to identify top transcription factor motifs enriched in day 0 samples (left) and day 28 samples (right) using the polyclonal ATAC-seq datasets (FIG. 6E). Day 28 specific peaks were enriched for the SOX17 motif, which is identical to the SOX4 motif (FIG. 15C), whereas day 0 peaks displayed no SOX enrichment. To investigate this further, the ATAC-seq signal at peaks with or without an underlying SOX4 motif were compared (FIG. 6F). Day 28 specific ATAC-seq peaks with a SOX4 motif display increased ATAC-seq signal (p=7.9e-07) but not ATAC-seq peaks that lacked a SOX4 motif, while day 0 samples showed no significant difference (p=0.09) (FIG. 6F, right). These results indicated that CAR T cells develop an opening of chromatin at SOX4 sites upon CAE. ATAC-seq peaks that did not change between day 0 and 28 (FIG. 6F, left) showed no specific enrichment for SOX4 motifs. Thus, SOX4 and ID3 were specifically expressed in the dysfunctional T cell clusters, were predicted to regulate DEGs upon CAE, and SOX4 motifs were enriched in chromatin opening under CAE conditions. It was concluded that these transcription factors are top candidates to regulate the dysfunction signature and the T-to-NK-like T cell transition that identified herein. Further, 18/30 of our dysfunction signature genes had chromatin opening at SOX4 motifs in day 28 CAE cells including AFAP1L2, CDK6, and CSF1 (FIGS. 6G-6I), and NK receptor genes KLRC1 and KLRB1 (FIGS. 23A and 23B). These results indicate that CAR T cells develop an opening of chromatin at SOX4 sites upon CAE.

### Example 9: Discussion

Recently several studies have suggested that T cell dysfunction is a major contributor to ineffective CAR T cell therapy in solid tumors. However, little is known about the mechanisms mediating loss of CAR T cell function. Herein it was examined how prolonged exposure to tumor antigen (CAE) in an in vitro model, as similarly encountered by CAR T cells in the TME, impacts the efficacy, surface expression, and phenotype of CAR T cells. The acquisition of a CAR T dysfunctional or exhaustion gene signature and the transcription factors that potentially regulate this transition was shown herein. Moreover, multiple mechanisms of CAR T dysfunction were identified and their relevance to patients treated with CAR T cell therapy was demonstrated.

It is widely accepted that TCR and CAR are internalized upon T cell activation, and later re-expressed on the cell surface. It was demonstrated herein that prolonged CAE leads to a significant decrease in the percentage of T cells that re-express CAR on the cell surface. As expected, in vitro CAR T cell efficacy declined in the models with diminished surface CAR expression. Importantly, this loss of surface CAR expression was observed in samples from clinical trial patients undergoing CAR T cell therapy, revealing that CAR internalization is an important factor limiting the therapeutic efficacy of M5 CAR T cells targeting solid tumors.

Although most CAR T cells do not re-express CAR on the cell surface after prolonged CAE, a small subpopulation retain surface CAR expression. Surprisingly, this population (surCARpos CD8+ CAR T cells) also displays dramatic reduction in effector function, revealing that loss of surface CAR T expression is not the only mechanism leading to loss of CAR T efficacy. To further investigate this, the phenotypic features of surCARpos CD8+ T cells from CAE were examined herein using genomic approaches. A CAR T cell dysfunction signature was identified that overlaps with signatures of T cell dysfunction or exhaustion in existing in vivo models including the LCMV mouse model of chronic viral infection and human CD8+ TILs isolated from HCC, CRC, NSCLC, and melanoma patients (Guo et al., (2018) Nat Med 24, 978-985; Li et al., (2019) Cell 176, 775-789.e718; Pauken et al., (2016) Science 354, 1160-1165; Zhang et al., (2018) Nature 564, 268-272; Zheng et al., (2017) Cell 169, 1342-1356.e1316). Strikingly, robust alignment was observed of the dysfunction gene signature identified in our in vitro CAR T CAE model with gene expression changes in NY-ESO TILs isolated from in vivo tumors compared to product T cells expressing NY-ESO-reactive TCRs; this important correlation suggests that the dysfunctional signature is relevant to gene engineered cell therapy, independent of whether CAR or TCR-mediated. Of note, the CAR T dysfunction signature was independent of CAR localization within the cell: gene expression values in surCARpos cells upon prolonged CAE strongly aligned with expression values observed for surCARneg cells. This congruence between the surCARpos and surCARneg gene signatures was further supported by the finding that CAR T cells impaired in the ability to express surface CAR after prolonged CAE re-gained surface CAR expression after rest. Moreover, these results underscored that there are additional mechanisms of dysfunction beyond CAR internalization, as cells with CAR on the surface are dysfunctional.

Another mechanism of CAR T cell dysfunction was identified whereby cells undergo a transition from T cells to NK-like T cells. The trivial possibility that the NK-like T cells expanded from the day 0 product was carefully ruled out, but instead showed that the elevated frequency of NK-like T cells during prolonged CAE resulted from a CD8+T-to-NK-like T cell transition. Results showed that these cell are distinct from CD1d-restricted invariant NKT (iNKT) cells and instead resemble NK-like T cells characterized by increased expression of NK related genes and proteins. These findings are supported by reports that CD8+ T cells acquire innate like characteristics by expressing NK receptors during chronic antigen exposure, and by observations of increased expression of NK receptors on tumor-infiltrating CD8+ T cells isolated from patients with hematological malignancy and solid tumors. Several studies have shown that NKG2A (which can be expressed on CD8+ T cells upon activation) acts as a novel immune checkpoint and that blocking the NKG2A receptor improves the efficacy of immunotherapies. Further, CD8+ cytotoxic T lymphocytes (CTLs) expressing cytotoxic granule proteins perforin, granzyme B, granulysin, and NK receptor NKG2C mediate TCR-dependent and independent anti-microbial activity. Interestingly, in addition to NK receptors, CAR T cells in the in vitro model express all three cytotoxic granule protein genes. Furthermore, NK transformation of CTLs has been observed in celiac disease. Together, these data support that NK-like T cells have an important role in immunity and that T cells can undergo a transition to NK-like T cells. Under prolonged CAE, CAR T cells both fail to re-express surface CAR and exhibit a significant decrease in the expression of genes involved in the antigen presentation pathway (see FIG. 2C). Without wishing to be bound by any specific theory, these conditions may select for T cells that transition to NK-like T cells because NK receptors provide needed signals required for T cell survival. Expression of the inhibitory NK receptors, such as CD94-NKG2A, KLRB1 (CD161), TIGIT, and inhibitory KIR may initially serve as a feedback mechanism to dampen excessive stimulatory signaling to avoid activation-induced cell death induced by TCR or CAR.

UMAP projection of single-cell gene expression data from CAE day 20 CAR transduced CD8+ T cells uncovered both non-dysfunctional and dysfunctional clusters. As expected, genes that define the dysfunctional clusters are not observed in the UMAP projection of single-cell gene data from day 0 product. The dysfunctional T cell clusters are defined by a robust gene expression signature that includes genes implicated in T cell exhaustion such as HAVCR2, LAYN, PHLDA1 and TNFRSF9 and new genes with no known connection to dysfunction including RGS16, SRGAP3, DUSP4, NDFIP2, and CD9. Interestingly, RGS16 plays an important role in T cell activation and attenuation of and CD9 is known as a key regulator of cell adhesion at the immune synapse, suggesting these molecules may be involved in dysfunction of T cell motility. NDFIP2 restricts effector function of CD4+ T cells and its homologue, NDFIP1, is associated with regulatory T cell stability; however, little is known about its function in CD8+ T cells. The human protein atlas shows SRGAP3, a Rho GTPase activating protein, is expressed more highly in NK cells than CD8+ T cells and thus it may be involved in the NK-like T cell transition. Importantly, SRGAP3 has no known function in NK, NK-like T, or T cells, making it an interesting candidate for further exploration. CAR expression was predominately detected in the dysfunctional clusters, with minimal expression in the non-dysfunctional clusters, indicating that prolonged CAE is driving the CAR T dysfunction or exhaustion phenotype. Monocle trajectory analysis was employed to group CAR T cell subsets on a continuum by their gene expression profiles and this showed a gradual progression with cells at the end of the trajectory expressing the highest levels of the dysfunction signature genes. The data confirm that T cells expressing CAR transition to a dysfunction or exhaustion phenotype during prolonged antigen stimulation.

The regulatory mechanisms driving CAR T cell dysfunction was further investigated. The transcription factors SOX4 and ID3 were specifically upregulated in prolonged CAE CAR T cells and were predicted to regulate the dysfunctional gene signature. Importantly, dysfunctional clusters in particular were associated with expression of NK receptors, SOX4 and ID3 transcription factors, and exhaustion-related genes. Additional support for co-regulation of exhaustion markers, ID3 and SOX4 transcription factors, and NK receptors genes was provided by an unbiased gene regulatory network analysis (PIDC, FIG. 3). The resulting matrix illustrated that many genes in the dysfunctional signature are grouped within the same community, suggesting that these genes are co-expressed in the same cells. ID3, which is a natural dominant negative helix-loop-helix (HLH) transcription factor, has a role in T cell differentiation. Further, ID3 is important for promoting the thymic development of bipotential NK/T progenitors to an NK cell fate and forced expression of ID3 blocks T cell and promotes NK cell development in a fetal thymic organ culture system. In addition, Prdm1 and Id3 expression distinguish distinct CD8+ T cell subsets in acute viral and bacterial infections and tumors. SOX4 has been shown to control thymic production of iNKT cells by inducing microRNA-181 (Mir181) to enhance TCR signaling. Data herein revealed that T cells undergo a transition to NK-like T cells and showed that ID3 and SOX4 transcription factors are involved in regulating the CAR T cell dysfunction signature, including the CD8+T-to-NK-like T cell transition.

In summary, the robust in vitro model of dysfunction in pancreatic cancer revealed multiple mechanisms of CAR T cell dysfunction and closely aligned with gene expression signatures of human TILs isolated from multiple cancer types. These findings provide a new perspective that suggests novel approaches for improving the efficacy of CAR T cell therapy in solid tumors. The model has the potential to be an inexpensive and reliable platform to identify and test factors that either prevent and/or reverse CAR T cell dysfunction with CAR and cancer type flexibility.

### Example 10: CRISPR Knockout of SOX4 and ID3

Using a multiomics approach coupled with phenotypic assays, a novel role for the transcription factors SOX4 and ID3 in CAR T cell dysfunction was uncovered. Dysfunctional CD8 CAR T cells undergo a post-thymic transition from CD8 T to NK-like T cells and express a specific gene expression signature that is regulated by SOX4 and ID3. Manipulating these factors in CAR T cells (through either overexpression or knock out) affects the efficacy of CAR T cell therapy. CRISPR mediated knock out of these factors affects the acquisition of the CAR T dysregulation signature and the associated NK-like T cell transition, thereby enhancing the function of CAR T cells. These factors are tested using the CAR T cell in vitro model whereby chronic antigen stimulation drives dysfunction. This is validated in animal models and then the approach (SOX4+/−ID3 knock out in CAR T cells) is used for therapeutic purposes in the clinic.

Further, the CAR T dysregulation signature and the T to NK-like T cell transition mediated by SOX4 and ID3 transcription factors also translates to synthetic TCR therapy. Knock out of these transcription factors will improve the therapeutic efficacy of TCRs engineered to recognize tumor antigens. In addition, the signature has significant overlap with exhausted T cells from a mouse model of chronic viral infection (LCMV), and therefore, SOX4/ID3 modulated T cells can also be used to treat chronic infections like HIV, EBV and CMV.

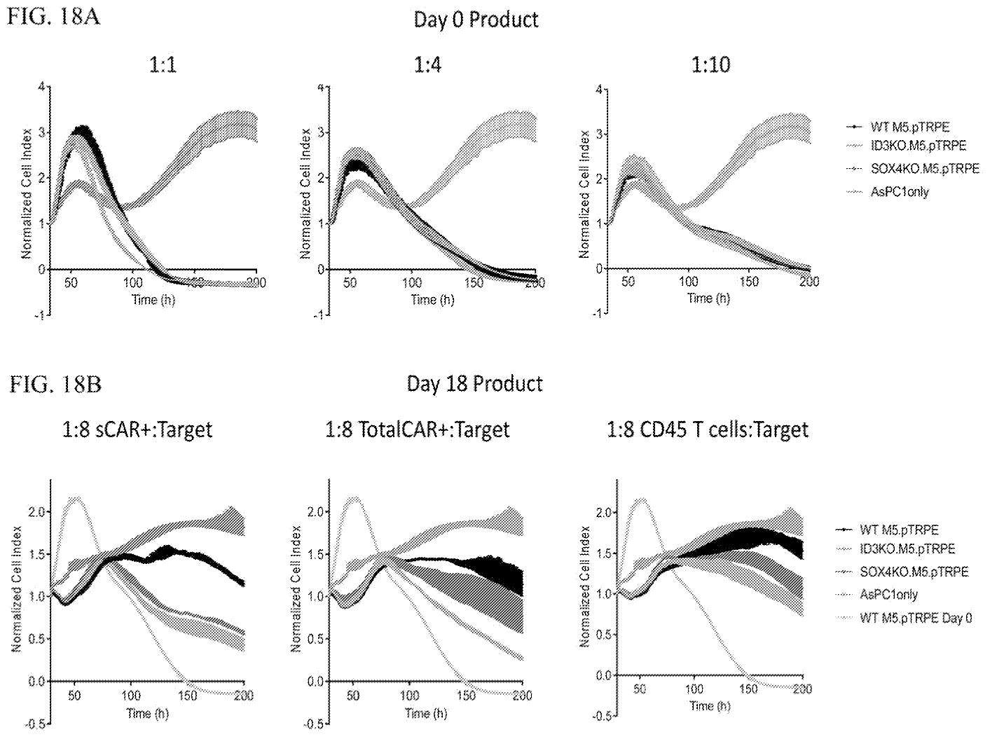

CRISPR-mediated knock out of ID3 and SOX4 does not modify CAR T cell killing efficiency on Day 0 product, but restores the killing ability of exhausted CAR T cells. Cell killing capacity of Day 0 product of ND539 MockM5.pTRPE, ID3KO.M5.pTRPE and SOX4.KO.M5.pTRPE CAR T cells against AsPC-1 cells measured by xCelligence is shown in FIG. 18A. Day 0 CAR T cells were seeded in three different surface CAR+:Target ratios. Negative control (AsPC-1 only) is shown in grey. Cell killing capacity of ND539 MockM5.pTRPE, ID3KO.M5.pTRPE and SOX4.KO.M5.pTRPE CAR T cells against AsPC-1 cells after 18 days of CAE measured by xCelligence is shown in FIG. 18B. Day 18 CAR T cells were seeded in 1:8 surface CAR+: target (left panel), 1:8 Total CAR+:Target (middle panel) and 1:8 CD45+:Target ratios. Negative control (AsPC-1 only) and positive control (Day 0 ND539 Mock M5.pTRPE product) are depicted.

### Example 10: Resting M5 CAR T and NY-ESO-1 TCR Specific T Cells Results in the Downregulation of 11/30 Dysfunction Signature Genes

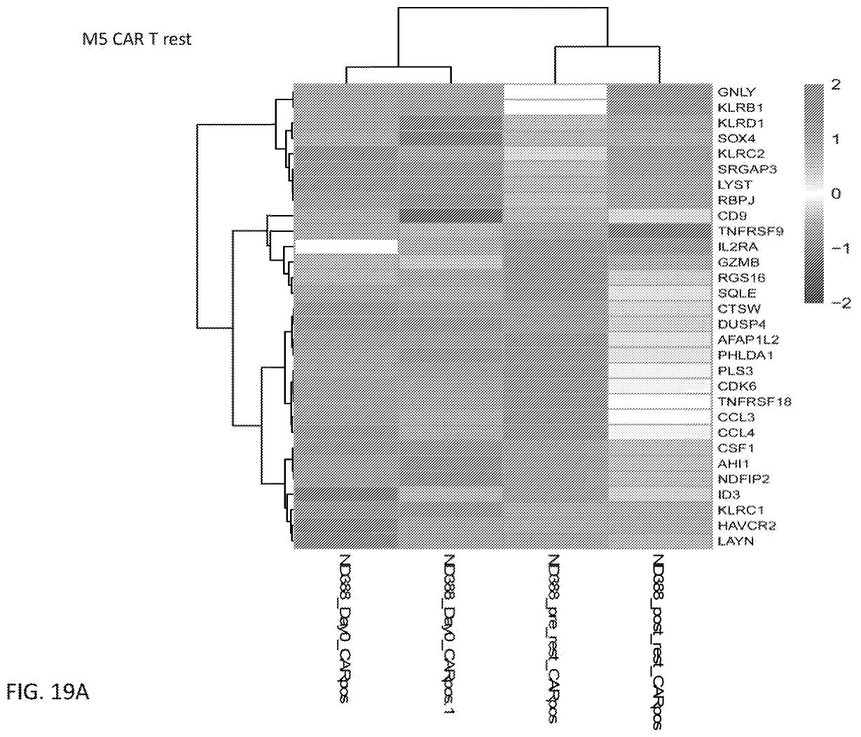

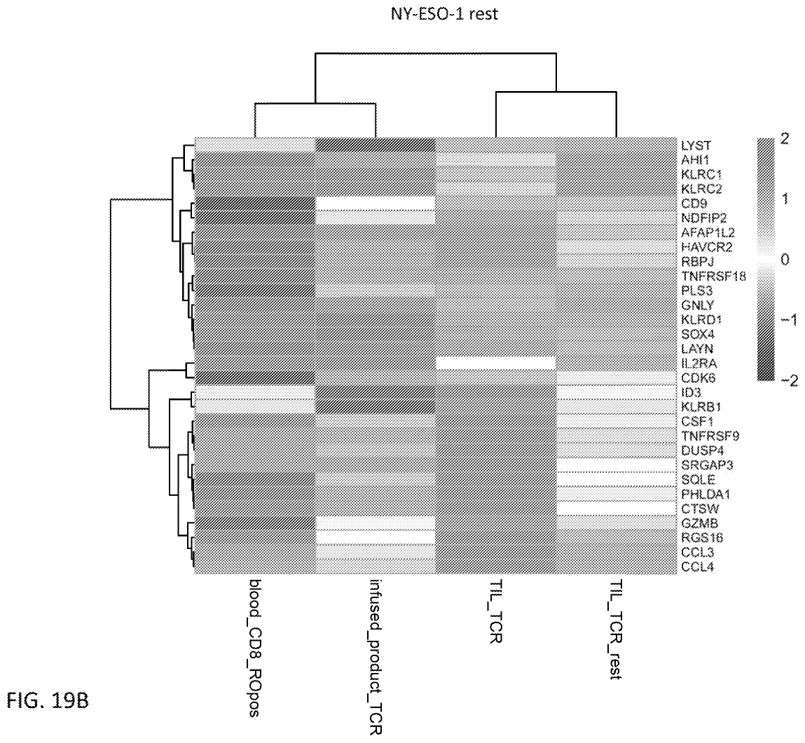

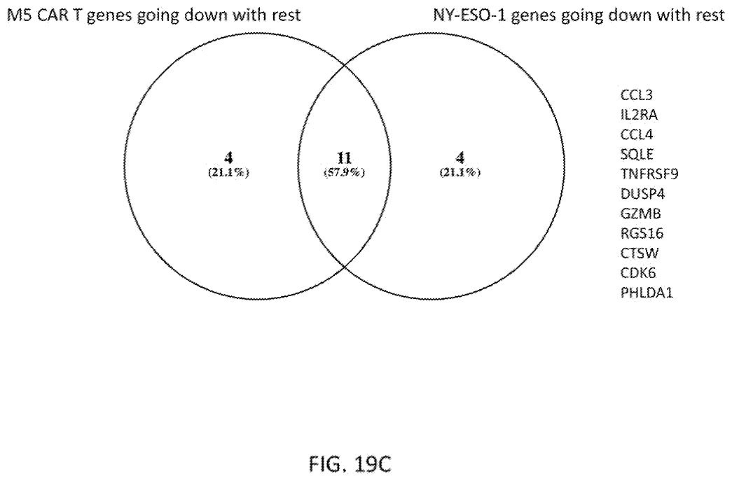

A heatmap of dysfunction signature genes (N=30) in Day 0 CAR T product, continuously stimulated M5 CAR T cells and rested M5 CAR T cells is shown in FIG. 19A. Resting the continuously stimulated CAR T cells for 24 hours with IL-15 improves cytotoxic function (see FIG. 1J). The rested CAR T cells downregulate 15/30 of the dysfunction signature genes (compare ND388_pre_rest_CARpos to ND388_post_rest_CARpos). Genes downregulated with rest are denoted by a black bracket on the right in FIG. 19A. A heatmap of dysfunction signature genes (N=30), in NY-ESO-1 reactive CD8+ TILs obtained from a lung xenograft tumor model without rest (TIL_TCR) and with rest (TIL_TCR_rest), along with controls including day 40 blood (CD8+CD45RO+ T cells) and day 0 infused product is shown in FIG. 19B. TILs were extracted 40 days following infusion and gene expression profiles (along with controls) were determined by microarray analysis. Rested NY-ESO-1 TCR specific TILs display improved cytotoxicity following rest (Moon et al., 2016) and downregulate 15/30 of the dysfunction signature genes (see black bracket on the right). FIG. 19C is a Venn diagram overlap of genes going down with rest in M5 CAR T cells (left) and NY-ESO-1 TCR specific T cells (right). 11/30 (37%) of the dysfunction signature genes go down in both models with rest.

### Example 11: Disruption of ID3 and SOX4 Improves CAR T Effector Function

To investigate whether ID3 and SOX4 regulate the dysfunction signature and T-to-NK-like T transition, as well as drive CAR T dysfunction, ID3 and SOX4 KO CAR T cells were generated using CRISPR-Cas9 (FIGS. 20A and 23C). The efficiency of KO cells in the day 0 product was validated (FIG. 23C). No differences in cytotoxicity (FIG. 23D) or T cell subset distribution (naive, effector, and memory populations) were observed at baseline between WT and KO day 0 CAR T cells (FIG. 23E); however, as expected, there were minor differences in T cell subsets between the CAR T donors.

To study the role of the transcription factors in driving CAR T dysfunction, WT, ID3 KO, and SOX4 KO CAR T cells were challenged with CAE for 20-28 days and their transcriptional profile and cytotoxic capacity was analyzed (FIG. 20B). Of note, day 0 and CAE conditions showed a similar KO efficiency, suggesting that there was no enrichment or depletion of SOX4 or ID3 KO cells during CAE (FIGS. 20C, 20D, and 23C). To identify whether the transcription factors regulate the NK phenotype and/or the dysfunction signature genes, scRNA-seq was performed. WT cells clustered predominantly on the right side, while ID3 and SOX4 KO cells clustered largely on the left (FIG. 20E). Interestingly, the KO cluster on the left was depleted of NK-like T cells (FIG. 20F) and overall, KO cells showed a significant reduction in the frequency of NK-like T cells compared to WT cells at day 24 (FIG. 20G). This finding was validated in an independent CAR T donor for ID3 KO cells at day 20 CAE (FIG. 23F).

A “dysfunction score” was calculated for each cell by taking the average expression level of the 30 genes in the signature. Importantly, cells that expressed the highest dysfunction score were clustered to the right (FIG. 20H), coincident with the cluster of NK-like T cells (FIG. 20F); overall, the KO conditions displayed a significant decrease in the dysfunction score per cell (FIG. 20I). This finding was reproduced in an independent CAR T donor for WT and ID3 KO conditions at day 20 CAE (FIG. 20J). A dot plot also revealed downregulation of the dysfunction signature in ID3 and SOX4 KO cells (FIG. 20K). Interestingly, significant loss of SOX4 expression was observed in the ID3 KO cells, suggesting that SOX4 is a putative ID3 target (FIG. 20L). Hence, the ID3 KO cells resembled a double KO as they lacked both ID3 and SOX4 expression. AFAP1L2 and CSF1 (genes upregulated in CAE) displayed chromatin opening in day 28 CAE cells at SOX4 motifs (see FIGS. 6G and 6I), and these genes were significantly downregulated in KO cells and are thus putative SOX4 target genes (FIGS. 20M and 20N). Of note, ID3 was significantly downregulated in SOX4 KO cells (FIG. 20O), although expression was not abrogated, suggesting ID3 may have additional transcriptional regulators. Select genes significantly downregulated in both KO conditions include LAYN, CD9, TNFRSF18, GNLY, and KLRC1 (FIGS. 20P-20T).

To determine whether KO of ID3 or SOX4 associated with increased effector function, cytotoxicity assays were performed following CAE with WT, ID3 KO, and SOX4 KO cells. Importantly, ID3 and SOX4 KO cells showed enhanced CAR T killing of tumor cells after CAE compared to WT cells (FIGS. 20U and 23G-23I).

### Example 12: The Role of ID3 and SOX4 in Driving CAR T Cell Dysfunction

One of the major challenges of CAR T cell therapy in solid tumors is to prevent T cell dysfunction, which is induced by a potent immunosuppressive microenvironment and by the continuous stimulation of the CAR T cells through its chimeric receptor. Since the mechanisms mediating dysfunction in CAR T cells remain poorly understood, the conditions of continuous stimulation of the CAR T cells were modeled in vitro in order to characterize the dysfunction phenotype of CAR T cells. In the model, healthy donor T cells expressing the anti-Mesothelin (M5) CAR are repeatedly stimulated with a mesothelin-expressing pancreatic cancer cell line (AsPC-1). This continuous antigen exposure (CAE) in vitro model leads to dysfunction of M5 CAR T cells, which recapitulates hallmark features of T cell exhaustion such as reduced proliferation capacity, and impaired cytotoxicity and cytokine production.

The CAR T cell dysfunction signature identified through the in vitro CAE model was regulated by the transcription factors ID3 and SOX4. By CRISPR-Cas9 technology, each transcription factor was disrupted individually in M5 CAR T cells. Importantly, such gene deletions didn't impact the T cell phenotype or killing potential of the manufactured product as compared with the WT (Mock) M5 CAR T cells, but provided resistance to dysfunction and enhanced tumor killing in the context of chronic antigen exposure.

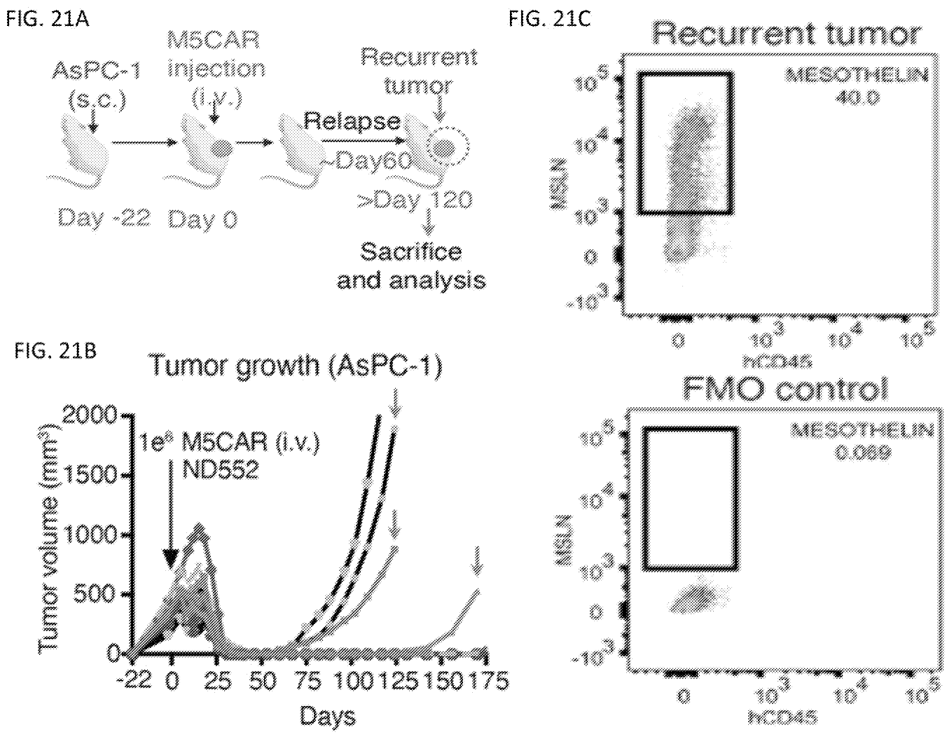

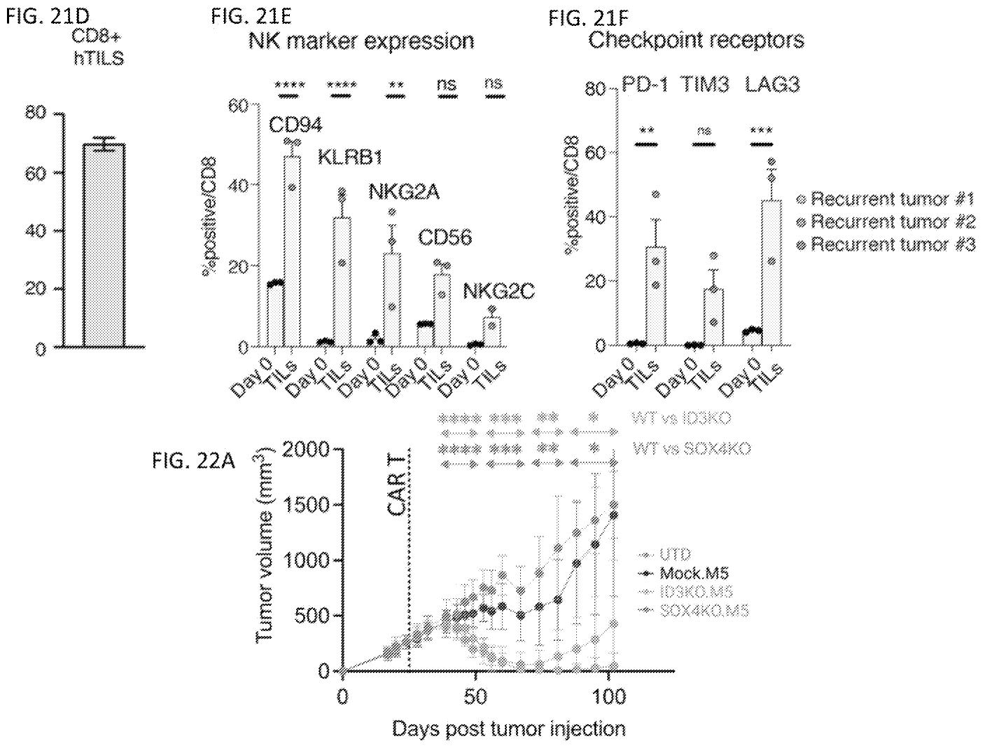

To confirm this phenotype was also observed in M5 CAR T cells in vivo, AsPC-1 tumor xenografts were generated then treated with M5 CAR T cells. The infused M5 CAR T cells were able to induce a potent antitumor response, eliminating large tumors within two weeks. However, two to four months after adoptive cell transfer, several of the cured mice relapsed at the same location, indicating that a small subset of AsPC-1 cells were able to regrow (FIGS. 21A and 21B). Although recurrent tumors expressed mesothelin (MSLN, FIG. 21C), the infiltrating CAR T cells exposed to CAE lost their ability to control the tumor growth. Interestingly, the M5 CAR T cells infiltrating the tumors were majority CD8+ CAR T cells (FIG. 21D) expressing key surface receptors of the dysfunction signature, such as high levels of NK receptors (FIG. 21D) and checkpoint receptors, unlike the day 0 CAR T product (FIG. 21E). This data aligns with the results on dysfunctional TCR T cells targeting the MHC class I epitope of NY-ESO-1, which also expressed the dysfunctional signature.

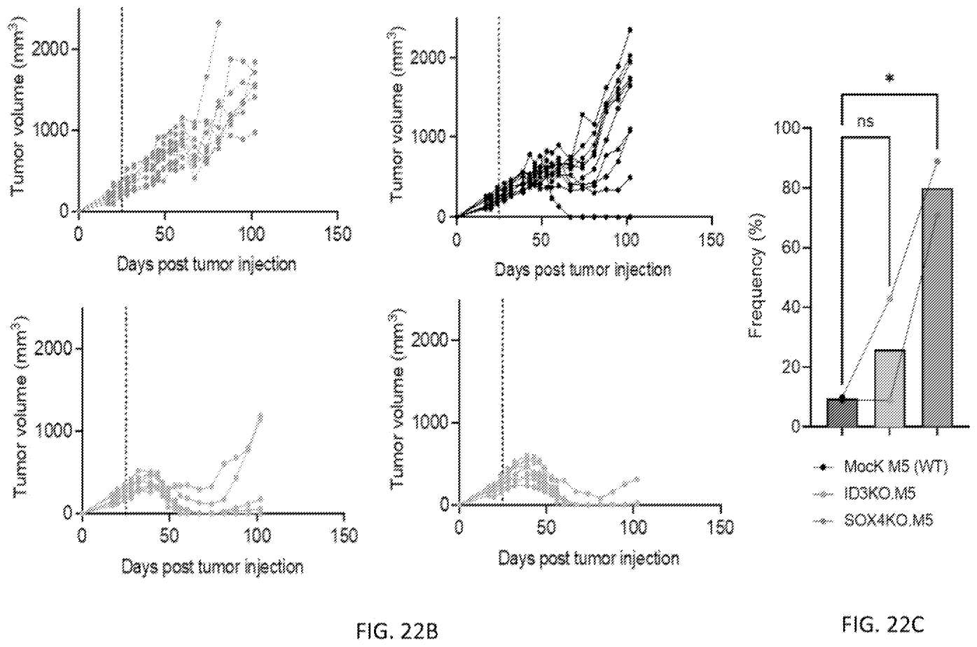

Moreover, to confirm that ID3 KO and SOX4 KO M5 CAR T cells have enhanced resistance to exhaustion and therefore improved tumor killing ability in vivo, the efficacy of KO M5 CAR T cells was characterized in AsPC-1 tumor xenografts. As shown below, SOX4 and ID3 KO M5 CAR T cells elicited a superior antitumor response compared to WT M5 CAR T cells, (FIGS. 22A and 22B). These results have been consistent in two different donors (FIG. 22C) at two different doses of CAR T cells, confirming a dose-dependent killing improvement over the WT M5 CAR control.

Taken together, the data suggest that ID3 and SOX4 regulate CAR T cell dysfunction in vitro and in vivo, and that such dysfunctional phenotypes can be overcome by CRISPR/Cas9 gene editing.

### Enumerated Embodiments

The following enumerated embodiments are provided, the numbering of which is not to be construed as designating levels of importance.

Embodiment 1 provides a modified immune cell or precursor cell thereof, comprising: a modification in an endogenous gene locus encoding SOX and/or ID3, wherein the modification is capable of downregulating gene expression of endogenous SOX and/or ID3.

Embodiment 2 provides the modified immune cell or precursor cell of embodiment 1, further comprising an exogenous T cell receptor (TCR) and/or chimeric antigen receptor (CAR) comprising affinity for an antigen on a target cell.

Embodiment 3 provides the modified immune cell or precursor cell of any preceding embodiment, wherein the modification is selected from the group consisting of a substitution, an insertion, a deletion, and an insertion/deletion.

Embodiment 4 provides the modified immune cell or precursor cell of any preceding embodiment, wherein the modification is mediated by a CRISPR system comprising a CRISPR nuclease and a guide RNA.

Embodiment 5 provides the modified immune cell or precursor cell of any preceding embodiment, wherein the modification is mediated by CRISPR/Cas9.

Embodiment 6 provides the modified immune cell or precursor cell of any preceding embodiment, wherein the guide RNA comprises a guide sequence that is sufficiently complementary with a target sequence in the endogenous gene locus encoding SOX and/or ID3.

Embodiment 7 provides the modified immune cell or precursor cell of any of embodiments 4-6, wherein the guide RNA comprises the nucleic acid sequence set forth in any one of SEQ ID NOs: 1-10.

Embodiment 8 provides the modified immune cell or precursor cell of embodiment 2, wherein the exogenous TCR is selected from the group consisting of a wild-type TCR, a high affinity TCR, and a chimeric TCR.

Embodiment 9 provides the modified immune cell or precursor cell of embodiment 2, wherein the exogenous CAR comprises an antigen binding domain, a transmembrane domain, and an intracellular domain.

Embodiment 10 provides the modified immune cell or precursor cell of embodiment 9, wherein the antigen binding domain is selected from the group consisting of an antibody, an scFv, and a Fab.

Embodiment 11 provides the modified immune cell or precursor cell of embodiment 2, wherein the exogenous CAR further comprises a hinge domain.

Embodiment 12 provides the modified immune cell or precursor cell of embodiment 11, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

Embodiment 13 provides the modified immune cell or precursor cell of embodiment 2, wherein the exogenous CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

Embodiment 14 provides the modified immune cell or precursor cell of embodiment 2, wherein the exogenous CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

Embodiment 15 provides the modified immune cell or precursor cell of embodiment 2, wherein the exogenous CAR comprises an intracellular domain comprising an intracellular domain selected from the group consisting of cytoplasmic signaling domains of a human CD3 zeta chain, FcyRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

Embodiment 16 provides the modified immune cell or precursor cell of embodiment 2, wherein the antigen on a target cell is a tumor associated antigen (TAA).

Embodiment 17 provides a modified immune cell or precursor cell thereof, comprising a nucleic acid capable of overexpressing endogenous SOX and/or ID3.

Embodiment 18 provides the modified immune cell or precursor cell of embodiment 17, further comprising an exogenous T cell receptor (TCR) and/or chimeric antigen receptor (CAR) comprising affinity for an antigen on a target cell.

Embodiment 19 provides the modified immune cell or precursor cell of embodiment 18, wherein the exogenous TCR is selected from the group consisting of a wild-type TCR, a high affinity TCR, and a chimeric TCR.

Embodiment 20 provides the modified immune cell or precursor cell of embodiment 18, wherein the exogenous CAR comprises an antigen binding domain, a transmembrane domain, and an intracellular domain.

Embodiment 21 provides the modified immune cell or precursor cell of embodiment 20, wherein the antigen binding domain is selected from the group consisting of an antibody, an scFv, and a Fab.

Embodiment 22 provides the modified immune cell or precursor cell of embodiment 20, wherein the exogenous CAR further comprises a hinge domain.

Embodiment 23 provides the modified immune cell or precursor cell of embodiment 22, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

Embodiment 24 provides the modified immune cell or precursor cell of embodiment 20, wherein the exogenous CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

Embodiment 25 provides the modified immune cell or precursor cell of embodiment 20, wherein the exogenous CAR comprises at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

Embodiment 26 provides the modified immune cell or precursor cell of embodiment 20, wherein the exogenous CAR comprises an intracellular domain comprising an intracellular domain selected from the group consisting of cytoplasmic signaling domains of a human CD3 zeta chain, FcyRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

Embodiment 27 provides the modified immune cell or precursor cell of embodiment 18, wherein the antigen on a target cell is a tumor associated antigen (TAA).

Embodiment 28 provides the modified immune cell or precursor cell of any preceding embodiment, wherein the modified cell is resistant to cell exhaustion and/or dysfunction.

Embodiment 29 provides the modified immune cell or precursor cell of any preceding embodiment, wherein the modified cell is an autologous cell.

Embodiment 30 provides the modified immune cell or precursor cell of any preceding embodiment, wherein the modified cell is a cell isolated from a human subject.

Embodiment 31 provides the modified immune cell or precursor cell of any preceding embodiment, wherein the modified cell is a modified T cell.

Embodiment 32 provides the modified immune cell or precursor cell of any preceding embodiment, wherein the modified cell is a modified T cell resistant to T cell exhaustion and/or T cell dysfunction.

Embodiment 33 provides a method for generating a modified immune cell or precursor cell thereof, comprising introducing into an immune or precursor cell a CRISPR system comprising one or more polypeptides and/or nucleic acids capable of downregulating gene expression of endogenous SOX and/or ID3; and introducing into the immune or precursor cell a nucleic acid encoding an exogenous T cell receptor (TCR) and/or chimeric antigen receptor (CAR), wherein the exogenous TCR and/or CAR comprises affinity for an antigen on a target cell.

Embodiment 34 provides a method for generating a modified immune cell or precursor cell thereof, comprising introducing into an immune or precursor cell a nucleic acid capable of over-expressing endogenous SOX and/or ID3; and introducing into the immune or precursor cell a nucleic acid encoding an exogenous T cell receptor (TCR) and/or chimeric antigen receptor (CAR), wherein the exogenous TCR and/or CAR comprises affinity for an antigen on a target cell.

Embodiment 35 provides the method of embodiment 33, wherein the one or more polypeptides and/or nucleic acids capable of downregulating gene expression of endogenous SOX and/or ID3 introduces a CRISPR-mediated modification in an endogenous gene locus encoding SOX and/or ID3.

Embodiment 36 provides the method of embodiment 33, wherein the modification is selected from the group consisting of a substitution, an insertion, a deletion, and an insertion/deletion.

Embodiment 37 provides the method of embodiment 33, wherein the CRISPR system comprises a CRISPR nuclease and a guide RNA.

Embodiment 38 provides the method of embodiment 37, wherein the CRISPR nuclease is Cas9.

Embodiment 39 provides the method of embodiment 37 or 38, wherein the CRISPR nuclease and the guide RNA comprise a ribonucleoprotein (RNP) complex.

Embodiment 40 provides the method of embodiment 39, wherein the RNP complex is introduced by electroporation.

Embodiment 41 provides the method of any one of embodiments 37-40, wherein the guide RNA comprises a guide sequence that is sufficiently complementary with a target sequence in the endogenous gene locus encoding SOX and/or ID3.

Embodiment 42 provides the method of embodiment 41, wherein the guide RNA comprises a nucleic acid sequence set forth in any one of SEQ ID NO: 1-10.

Embodiment 43 provides the method of any preceding embodiment, wherein the nucleic acid encoding an exogenous TCR and/or CAR is introduced via viral transduction.

Embodiment 44 provides the method of embodiment 43, wherein the viral transduction comprises contacting the immune or precursor cell with a viral vector comprising the nucleic acid encoding an exogenous TCR and/or CAR.

Embodiment 45 provides the method of embodiment 44, wherein the viral vector is selected from the group consisting of a retroviral vector, a lentiviral vector, an adenoviral vector, and an adeno-associated viral (AAV) vector.

Embodiment 46 provides the method of embodiment 44, wherein the viral vector is a lentiviral vector.

Embodiment 47 provides the method of embodiment 43, wherein the viral transduction comprises contacting the immune or precursor cell with a viral vector comprising the nucleic acid encoding an exogenous TCR and/or CAR.

Embodiment 48 provides a method of treating a disease or disorder in a subject in need thereof, comprising administering to the subject the modified immune or precursor cell of any of embodiments 1-32, or a modified immune or precursor cell generated by the method of any of embodiments 33-47.

Embodiment 49 provides a method of treating a disease or disorder in a subject in need thereof, comprising administering to the subject a modified T cell comprising a CRISPR-mediated modification in an endogenous gene locus encoding SOX and/or ID3, wherein the modification is capable of downregulating gene expression of endogenous SOX and/or ID3; and an exogenous T cell receptor (TCR) and/or chimeric antigen receptor (CAR) comprising affinity for an antigen on a target cell.

Embodiment 50 provides the method of treating a disease or disorder in a subject in need thereof, comprising administering to the subject a modified T cell comprising a modification in an endogenous gene locus encoding SOX and/or ID3, wherein the modification is capable of over-expressing endogenous SOX and/or ID3; and an exogenous T cell receptor (TCR) and/or chimeric antigen receptor (CAR) comprising affinity for an antigen on a target cell.

Embodiment 51 provides the method of claims 48-50, wherein the antigen on a target cell is a tumor associated antigen (TAA).

Embodiment 52 provides the method of any one of embodiments 48-51, wherein the disease or disorder is cancer.

Embodiment 53 provides the method of embodiment 52, wherein the cancer comprises a solid tumor.

Embodiment 54 provides the method of any one of embodiments 48-50, wherein the disease or disorder is a chronic infection.

Embodiment 55 provides the method of embodiment 54, wherein the chronic infection is selected from the group consisting of HIV, EBV, CMV, LCMV.

Embodiment 56 provides the method of any one of embodiments 48-55, wherein the modified T cell is human.

Embodiment 57 provides the method of any one of embodiments 48-56, wherein the modified T cell is autologous.

Embodiment 59 provides the method of any one of embodiments 48-57, wherein the subject is human.

Embodiment 59 provides a method of assessing T cell dysfunction in a subject, the method comprising measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY, wherein when at least 11 of the genes are upregulated, the T cell is dysfunctional.

Embodiment 60 provides the method of embodiment 59, wherein the T cell comprises a CAR.

Embodiment 61 provides the method of embodiment 59, wherein the T cell comprises an engineered TCR.

Embodiment 62 provides the method of embodiment 59 or 60, wherein the CAR or TCR is capable of binding a tumor associated antigen (TAA).

Embodiment 63 provides a method for treating cancer in a subject in need thereof, the method comprising: i) administering a CAR T cell therapy to the subject, and ii) measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY, wherein when at least 11 of the genes are upregulated, the CAR T cells are deemed dysfunctional and an alternative therapy is administered.

Embodiment 64 provides a method of treating cancer in a subject in need thereof, the method comprising: i) administering to the subject a therapy comprising a T cell comprising an engineered TCR capable of binding a tumor associated antigen (TAA), and ii) measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY, wherein when at least 11 of the genes are upregulated, the T cells are deemed dysfunctional and an alternative therapy is administered.

Embodiment 65 provides a method of treating a disease, disorder, or chronic infection in a subject in need thereof, the method comprising: i) administering to the subject a T cell therapy, and ii) measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY, wherein when at least 11 of the genes are upregulated, the cells are deemed dysfunctional and an alternative therapy is administered.

Embodiment 66 provides the method of embodiment 65, wherein the chronic infection is selected from the group consisting of HIV, EBV and CMV.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A modified immune cell or precursor cell thereof, comprising:
a modification in an endogenous gene locus encoding SOX and/or ID3, wherein the modification is capable of downregulating gene expression of endogenous SOX and/or ID3.

2. The modified immune cell or precursor cell of claim 1, further comprising an exogenous T cell receptor (TCR) and/or chimeric antigen receptor (CAR) comprising affinity for an antigen on a target cell.

3. The modified immune cell or precursor cell of claim 1, wherein the modification is selected from the group consisting of a substitution, an insertion, a deletion, and an insertion/deletion.

4. The modified immune cell or precursor cell of claim 1, wherein the modification is mediated by a CRISPR system comprising a CRISPR nuclease and a guide RNA.

5. The modified immune cell or precursor cell of claim 1, wherein the modification is mediated by CRISPR/Cas9.

6. The modified immune cell or precursor cell of claim 4, wherein the guide RNA comprises a guide sequence that is sufficiently complementary with a target sequence in the endogenous gene locus encoding SOX and/or ID3.

7. The modified immune cell or precursor cell of claim 4, wherein the guide RNA comprises the nucleic acid sequence set forth in any one of SEQ ID NOs: 1-10.

8. The modified immune cell or precursor cell of claim 2, wherein the exogenous TCR is selected from the group consisting of a wild-type TCR, a high affinity TCR, and a chimeric TCR.

9. The modified immune cell or precursor cell of claim 2, wherein the exogenous CAR comprises an antigen binding domain, a transmembrane domain, and an intracellular domain.

10. The modified immune cell or precursor cell of claim 9, wherein the antigen binding domain is selected from the group consisting of an antibody, an scFv, and a Fab.

11. The modified immune cell or precursor cell of claim 2, wherein the exogenous CAR further comprises a hinge domain.

12. The modified immune cell or precursor cell of claim 11, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

13. The modified immune cell or precursor cell of claim 2, wherein the exogenous CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

14. The modified immune cell or precursor cell of claim 2, wherein the exogenous CAR comprises an intracellular domain comprising at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

15. The modified immune cell or precursor cell of claim 2, wherein the exogenous CAR comprises an intracellular domain comprising an intracellular signaling domain selected from the group consisting of cytoplasmic signaling domains of a human CD3 zeta chain, FcyRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

16. The modified immune cell or precursor cell of claim 2, wherein the antigen on a target cell is a tumor associated antigen (TAA).

17. A modified immune cell or precursor cell thereof, comprising:
a nucleic acid capable of overexpressing endogenous SOX and/or ID3.

18. The modified immune cell or precursor cell of claim 17, further comprising an exogenous T cell receptor (TCR) and/or chimeric antigen receptor (CAR) comprising affinity for an antigen on a target cell.

19. The modified immune cell or precursor cell of claim 18, wherein the exogenous TCR is selected from the group consisting of a wild-type TCR, a high affinity TCR, and a chimeric TCR.

20. The modified immune cell or precursor cell of claim 18, wherein the exogenous CAR comprises an antigen binding domain, a transmembrane domain, and an intracellular domain.

21. The modified immune cell or precursor cell of claim 20, wherein the antigen binding domain is selected from the group consisting of an antibody, an scFv, and a Fab.

22. The modified immune cell or precursor cell of claim 20, wherein the exogenous CAR further comprises a hinge domain.

23. The modified immune cell or precursor cell of claim 22, wherein the hinge domain is selected from the group consisting of an Fc fragment of an antibody, a hinge region of an antibody, a CH2 region of an antibody, a CH3 region of an antibody, an artificial hinge domain, a hinge comprising an amino acid sequence of CD8, or any combination thereof.

24. The modified immune cell or precursor cell of claim 20, wherein the exogenous CAR comprises a transmembrane domain selected from the group consisting of an artificial hydrophobic sequence and transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

25. The modified immune cell or precursor cell of claim 20, wherein the exogenous CAR comprises an intracellular domain comprising at least one co-stimulatory domain selected from the group consisting of co-stimulatory domains of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3.

26. The modified immune cell or precursor cell of claim 20, wherein the exogenous CAR comprises an intracellular domain comprising an intracellular signaling domain selected from the group consisting of cytoplasmic signaling domains of a human CD3 zeta chain, FcyRIII, FcsRI, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

27. The modified immune cell or precursor cell of claim 18, wherein the antigen on a target cell is a tumor associated antigen (TAA).

28. The modified immune cell or precursor cell of claim 1, wherein the modified cell is resistant to cell exhaustion and/or dysfunction.

29. The modified immune cell or precursor cell of claim 1, wherein the modified cell is an autologous cell.

30. The modified immune cell or precursor cell of claim 1, wherein the modified cell is a cell isolated from a human subject.

31. The modified immune cell or precursor cell of claim 1, wherein the modified cell is a modified T cell.

32. The modified immune cell or precursor cell of claim 1, wherein the modified cell is a modified T cell resistant to T cell exhaustion and/or T cell dysfunction.

33. A method for generating a modified immune cell or precursor cell thereof, comprising:
introducing into an immune or precursor cell a CRISPR system comprising one or more polypeptides and/or nucleic acids capable of downregulating gene expression of endogenous SOX and/or ID3; and
introducing into the immune or precursor cell a nucleic acid encoding an exogenous T cell receptor (TCR) and/or chimeric antigen receptor (CAR), wherein the exogenous TCR and/or CAR comprises affinity for an antigen on a target cell.

34. A method for generating a modified immune cell or precursor cell thereof, comprising:
introducing into an immune or precursor cell a nucleic acid capable of over-expressing endogenous SOX and/or ID3; and
introducing into the immune or precursor cell a nucleic acid encoding an exogenous T cell receptor (TCR) and/or chimeric antigen receptor (CAR), wherein the exogenous TCR and/or CAR comprises affinity for an antigen on a target cell.

35. The method of claim 33, wherein the one or more polypeptides and/or nucleic acids capable of downregulating gene expression of endogenous SOX and/or ID3 introduces a CRISPR-mediated modification in an endogenous gene locus encoding SOX and/or ID3.

36. The method of claim 33, wherein the modification is selected from the group consisting of a substitution, an insertion, a deletion, and an insertion/deletion.

37. The method of claim 33, wherein the CRISPR system comprises a CRISPR nuclease and a guide RNA.

38. The method of claim 37, wherein the CRISPR nuclease is Cas9.

39. The method of claim 37, wherein the CRISPR nuclease and the guide RNA comprise a ribonucleoprotein (RNP) complex.

40. The method of claim 39, wherein the RNP complex is introduced by electroporation.

41. The method of claim 37, wherein the guide RNA comprises a guide sequence that is sufficiently complementary with a target sequence in the endogenous gene locus encoding SOX and/or ID3.

42. The method of claim 41, wherein the guide RNA comprises a nucleic acid sequence set forth in any one of SEQ ID NO: 1-10.

43. The method of claim 33, wherein the nucleic acid encoding an exogenous TCR and/or CAR is introduced via viral transduction.

44. The method of claim 43, wherein the viral transduction comprises contacting the immune or precursor cell with a viral vector comprising the nucleic acid encoding an exogenous TCR and/or CAR.

45. The method of claim 44, wherein the viral vector is selected from the group consisting of a retroviral vector, a lentiviral vector, an adenoviral vector, and an adeno-associated viral (AAV) vector.

46. The method of claim 44, wherein the viral vector is a lentiviral vector.

47. (canceled)

48. A method of treating a disease or disorder in a subject in need thereof, the method comprising administering to the subject the modified immune or precursor cell of claim 1.

49. A method of treating a disease or disorder in a subject in need thereof, the method comprising administering to the subject a modified T cell comprising:
a CRISPR-mediated modification in an endogenous gene locus encoding SOX and/or ID3, wherein the modification is capable of downregulating gene expression of endogenous SOX and/or ID3; and
an exogenous T cell receptor (TCR) and/or chimeric antigen receptor (CAR) comprising affinity for an antigen on a target cell.

50. A method of treating a disease or disorder in a subject in need thereof, the method comprising administering to the subject a modified T cell comprising:
a modification in an endogenous gene locus encoding SOX and/or ID3, wherein the modification is capable of over-expressing endogenous SOX and/or ID3; and
an exogenous T cell receptor (TCR) and/or chimeric antigen receptor (CAR) comprising affinity for an antigen on a target cell.

51. The method of claim 49, wherein the antigen on a target cell is a tumor associated antigen (TAA).

52. The method of claim 49, wherein the disease or disorder is cancer.

53. The method of claim 52, wherein the cancer comprises a solid tumor.

54. The method of claim 49, wherein the disease or disorder is a chronic infection.

55. The method of claim 54, wherein the chronic infection is selected from the group consisting of HIV, EBV, CMV, LCMV.

56. The method of claim 49, wherein the modified T cell is a human T cell.

57. The method of claim 49, wherein the modified T cell is autologous.

58. The method of claim 49, wherein the subject is human.

59. A method of assessing T cell dysfunction in a subject, the method comprising measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY,
wherein when at least 11 of the genes are upregulated, the T cell is dysfunctional.

60. The method of claim 59, wherein the T cell comprises a CAR.

61. The method of claim 59, wherein the T cell comprises an engineered TCR.

62. The method of claim 59, wherein the CAR is capable of binding a tumor associated antigen (TAA).

63. A method for treating cancer in a subject in need thereof, the method comprising:
i) administering a CAR T cell therapy to the subject, and
ii) measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY,
wherein when at least 11 of the genes are upregulated, the CAR T cells are deemed dysfunctional and an alternative therapy is administered.

64. A method of treating cancer in a subject in need thereof, the method comprising:
i) administering to the subject a therapy comprising a T cell comprising an engineered TCR capable of binding a tumor associated antigen (TAA), and
ii) measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY,
wherein when at least 11 of the genes are upregulated, the T cells are deemed dysfunctional and an alternative therapy is administered.

65. A method of treating a disease, disorder, or chronic infection in a subject in need thereof, the method comprising:
i) administering to the subject a T cell therapy, and
ii) measuring a panel of genes in a sample from the subject, wherein the panel of genes is selected from the group consisting of HAVCR2, RGS16, LAYN, SRGAP3, DUSP4, CSF1, TNFRSF9, LYST, TNFRSF18, NDFIP2, SQLE, ID3, SOX4, CD9, PHLDA1, CCL3, CCL4, KLRC1, KLRD1, KLRB1, KLRC2, CDK6, PLS3, AFAP1L2, CTSW, IL2RA, AHI1, RBPJ, GZMB, and GNLY,
wherein when at least 11 of the genes are upregulated, the cells are deemed dysfunctional and an alternative therapy is administered.

66. The method of claim 65, wherein the chronic infection is selected from the group consisting of HIV, EBV and CMV.
